SEQUENCE LISTING (640 sequences)

SEQ ID NO: 1 - protein, 18 aa, Artificial Sequence
  A synthetic peptide
HVRRRRGTFKRSKLRARD

SEQ ID NO: 2 - protein, 16 aa, Artificial Sequence
  A synthetic peptide
KKKKRASFKRKSSKKG

SEQ ID NO: 3 - protein, 13 aa, Artificial Sequence
  A synthetic peptide
NRKKKRTSFKRKA

SEQ ID NO: 4 - protein, 15 aa, Artificial Sequence
  A synthetic peptide
KFARKSTRRSIRLPE

SEQ ID NO: 5 - protein, 16 aa, Artificial Sequence
  A synthetic peptide
RQRKRKLSFRRRTDKD

SEQ ID NO: 6 - protein, 14 aa, Artificial Sequence
  A synthetic peptide
PRLIRRGSKKRPAR

SEQ ID NO: 7 - protein, 17 aa, Artificial Sequence
  A synthetic peptide
RKIPKRPGSVHRTPSRQ

SEQ ID NO: 8 - protein, 15 aa, Artificial Sequence
  A synthetic peptide
AARKKRISVKKKQEQ

SEQ ID NO: 9 - protein, 15 aa, Artificial Sequence
  A synthetic peptide
QKKSRLRRRASQLKI

SEQ ID NO: 10 - protein, 14 aa, Artificial Sequence
  A synthetic peptide
AQIVKRASLKRGKQ

SEQ ID NO: 11 - protein, 15 aa, Artificial Sequence
  A synthetic peptide
KKKFRTPSFLKKSKK

SEQ ID NO: 12 - protein, 15 aa, Artificial Sequence
  A synthetic peptide
KKKKKRFSFKKSFKL

SEQ ID NO: 13 - protein, 14 aa, Artificial Sequence
  A synthetic peptide
WKGKRRSKARKKRK

SEQ ID NO: 14 - protein, 13 aa, Artificial Sequence
  A synthetic peptide
EYLERRASRRRAV

SEQ ID NO: 15 - protein, 18 aa, Artificial Sequence
  A synthetic peptide
RGFLRSASLGRRASFHLE

SEQ ID NO: 16 - protein, 14 aa, Artificial Sequence
  A synthetic peptide
DGQKRKKSLRKKLD

SEQ ID NO: 17 - protein, 15 aa, Artificial Sequence
  A synthetic peptide
AGWRKKTSFRKPKED

SEQ ID NO: 18 - protein, 20 aa, Artificial Sequence
  A synthetic peptide
KKRFSFKKSFKLSGFSFKKN

SEQ ID NO: 19 - protein, 13 aa, Artificial Sequence
  A synthetic peptide
AGSFKRNSIKKIV

SEQ ID NO: 20 - protein, 13 aa, Artificial Sequence
  A synthetic peptide
GAPPRRSSIRNAH

SEQ ID NO: 21 - protein, 15 aa, Artificial Sequence
  A synthetic peptide
KLAVGRHSFSRRSGV

SEQ ID NO: 22 - protein, 17 aa, Artificial Sequence
  A synthetic peptide
LLKKRDSFRTPRDSKLE

SEQ ID NO: 23 - protein, 15 aa, Artificial Sequence
  A synthetic peptide
QKRHARVTVKYDRRE

SEQ ID NO: 24 - protein, 16 aa, Artificial Sequence
  A synthetic peptide
EKIKRSSLKKVDSLKK

SEQ ID NO: 25 - protein, 15 aa, Artificial Sequence
  A synthetic peptide
EILSRRPSYRKILND

SEQ ID NO: 26 - protein, 13 aa, Artificial Sequence
  A synthetic peptide
ALRRPSLRREADD

SEQ ID NO: 27 - protein, 14 aa, Artificial Sequence
  A synthetic peptide
KKRKKKSSKSLAHA

SEQ ID NO: 28 - protein, 15 aa, Artificial Sequence
  A synthetic peptide
KRPGKKGSNKRPGKR

SEQ ID NO: 29 - protein, 15 aa, Artificial Sequence
  A synthetic peptide
RKNDRKKRYTVVGNP

SEQ ID NO: 30 - protein, 15 aa, Artificial Sequence
  A synthetic peptide
KEVVRTDSLKGRRGR

SEQ ID NO: 31 - protein, 20 aa, Artificial Sequence
  A synthetic peptide
RKKRKKKSSKSLAHAGVALA

SEQ ID NO: 32 - protein, 15 aa, Artificial Sequence
  A synthetic peptide
KATTKKRTLRKNDRK

SEQ ID NO: 33 - protein, 15 aa, Artificial Sequence
  A synthetic peptide
QQKIRKYTMRRLLQE

SEQ ID NO: 34 - protein, 12 aa, Artificial Sequence
  A synthetic peptide
EGGDRRASGRRK

SEQ ID NO: 35 - protein, 15 aa, Artificial Sequence
  A synthetic peptide
GLLDRKGSWKKLDDM

SEQ ID NO: 36 - protein, 15 aa, Artificial Sequence
  A synthetic peptide
GENVLKKSMKSRVKG

SEQ ID NO: 37 - protein, 15 aa, Artificial Sequence
  A synthetic peptide
AYIERMNSIHRDLRA

SEQ ID NO: 38 - protein, 15 aa, Artificial Sequence
  A synthetic peptide
NYLRRRLSDSNFMAN

SEQ ID NO: 39 - protein, 13 aa, Artificial Sequence
  A synthetic peptide
LLGSGKVTDRKAL

SEQ ID NO: 40 - protein, 15 aa, Artificial Sequence
  A synthetic peptide
NMEAKKLSKDRMKKY

SEQ ID NO: 41 - protein, 13 aa, Artificial Sequence
  A synthetic peptide
FVHQASFKFGQGD

SEQ ID NO: 42 - protein, 16 aa, Artificial Sequence
  A synthetic peptide
QPEGLRSLKKPDRKKR

SEQ ID NO: 43 - protein, 17 aa, Artificial Sequence
  A synthetic peptide
AWVTVHEKKSSRKSEYL

SEQ ID NO: 44 - protein, 14 aa, Artificial Sequence
  A synthetic peptide
VLAKKGTSKTPVPE

SEQ ID NO: 45 - protein, 15 aa, Artificial Sequence
  A synthetic peptide
VFREHQRSGSYHVRE

SEQ ID NO: 46 - protein, 14 aa, Artificial Sequence
  A synthetic peptide
GQAWGRQSPRRLED

SEQ ID NO: 47 - protein, 15 aa, Artificial Sequence
  A synthetic peptide
ARIIGEKSFRRSVVG

SEQ ID NO: 48 - protein, 13 aa, Artificial Sequence
  A synthetic peptide
AVNSRRRAGQKKK

SEQ ID NO: 49 - protein, 15 aa, Artificial Sequence
  A synthetic peptide
VQQLLRSSNRRLEQL

SEQ ID NO: 50 - protein, 15 aa, Artificial Sequence
  A synthetic peptide
ENLRRVATDRRHLGH

SEQ ID NO: 51 - protein, 16 aa, Artificial Sequence
  A synthetic peptide
DLLGKKVSTKTLSEDD

SEQ ID NO: 52 - protein, 15 aa, Artificial Sequence
  A synthetic peptide
HKHSPEKRGSERKEG

SEQ ID NO: 53 - protein, 15 aa, Artificial Sequence
  A synthetic peptide
AKNLKTLQKRDSFIG

SEQ ID NO: 54 - protein, 15 aa, Artificial Sequence
  A synthetic peptide
ENLRKVTTDKKSLAY

SEQ ID NO: 55 - protein, 14 aa, Artificial Sequence
  A synthetic peptide
DDMEHKTLKITDFG

SEQ ID NO: 56 - protein, 15 aa, Artificial Sequence
  A synthetic peptide
EARLGAASLKFGARD

SEQ ID NO: 57 - protein, 15 aa, Artificial Sequence
  A synthetic peptide
KNVVKLLSSRRTQDR

SEQ ID NO: 58 - protein, 13 aa, Artificial Sequence
  A synthetic peptide
RVKLGTLRRPEGP

SEQ ID NO: 59 - protein, 12 aa, Artificial Sequence
  A synthetic peptide
PVNKRSKYTMMK

SEQ ID NO: 60 - protein, 15 aa, Artificial Sequence
  A synthetic peptide
LRRKHLGTLNFGGIR

SEQ ID NO: 61 - protein, 15 aa, Artificial Sequence
  A synthetic peptide
VDNILKKSNKKLEEL

SEQ ID NO: 62 - protein, 15 aa, Artificial Sequence
  A synthetic peptide
AVRDMRQTVAVGVIK

SEQ ID NO: 63 - protein, 15 aa, Artificial Sequence
  A synthetic peptide
QRQERIFSKRRGQDF

SEQ ID NO: 64 - protein, 17 aa, Artificial Sequence
  A synthetic peptide
ALRAPKPTLRYFTTERF

SEQ ID NO: 65 - protein, 15 aa, Artificial Sequence
  A synthetic peptide
IKVTHKATGKVMVMK

SEQ ID NO: 66 - protein, 15 aa, Artificial Sequence
  A synthetic peptide
GFAKKIGSGQKTWTF

SEQ ID NO: 67 - protein, 15 aa, Artificial Sequence
  A synthetic peptide
AINSRETMFHKERFK

SEQ ID NO: 68 - protein, 15 aa, Artificial Sequence
  A synthetic peptide
RGEGHKPSIAHRDFK

SEQ ID NO: 69 - protein, 16 aa, Artificial Sequence
  A synthetic peptide
LALTARESSVRSGGAG

SEQ ID NO: 70 - protein, 13 aa, Artificial Sequence
  A synthetic peptide
HERKGSDKRGDNQ

SEQ ID NO: 71 - protein, 19 aa, Artificial Sequence
  A synthetic peptide
RRRQKRRTGALVLSRGGKR

SEQ ID NO: 72 - protein, 21 aa, Artificial Sequence
  A synthetic peptide
LTDPKEDPIYDEPEGLAPVPG

SEQ ID NO: 73 - protein, 15 aa, Artificial Sequence
  A synthetic peptide
IDYYKKTTNGRLPVK

SEQ ID NO: 74 - protein, 15 aa, Artificial Sequence
  A synthetic peptide
IDYYKKTSNGRLPVK

SEQ ID NO: 75 - protein, 15 aa, Artificial Sequence
  A synthetic peptide
EEAEHKATKARLADK

SEQ ID NO: 76 - protein, 18 aa, Artificial Sequence
  A synthetic peptide
HVRRRRGTFKRSKLRARD

SEQ ID NO: 77 - protein, 16 aa, Artificial Sequence
  A synthetic peptide
KKKKRASFKRKSSKKG

SEQ ID NO: 78 - protein, 15 aa, Artificial Sequence
  A synthetic peptide
KFARKSTRRSIRLPE

SEQ ID NO: 79 - protein, 13 aa, Artificial Sequence
  A synthetic peptide
NRKKKRTSFKRKA

SEQ ID NO: 80 - protein, 15 aa, Artificial Sequence
  A synthetic peptide
KKKKKRFSFKKSFKL

SEQ ID NO: 81 - protein, 16 aa, Artificial Sequence
  A synthetic peptide
KRERKTSSKSSVRKRR

SEQ ID NO: 82 - protein, 14 aa, Artificial Sequence
  A synthetic peptide
PRLIRRGSKKRPAR

SEQ ID NO: 83 - protein, 17 aa, Artificial Sequence
  A synthetic peptide
RKIPKRPGSVHRTPSRQ

SEQ ID NO: 84 - protein, 15 aa, Artificial Sequence
  A synthetic peptide
QKKSRLRRRASQLKI

SEQ ID NO: 85 - protein, 13 aa, Artificial Sequence
  A synthetic peptide
RFARKGSLRQKNV

SEQ ID NO: 86 - protein, 16 aa, Artificial Sequence
  A synthetic peptide
RQRKRKLSFRRRTDKD

SEQ ID NO: 87 - protein, 17 aa, Artificial Sequence
  A synthetic peptide
RQGKRKTSIKRDTVNPL

SEQ ID NO: 88 - protein, 17 aa, Artificial Sequence
  A synthetic peptide
KKPFKLSGLSFKRNRKE

SEQ ID NO: 89 - protein, 13 aa, Artificial Sequence
  A synthetic peptide
EYLERRASRRRAV

SEQ ID NO: 90 - protein, 14 aa, Artificial Sequence
  A synthetic peptide
AQIVKRASLKRGKQ

SEQ ID NO: 91 - protein, 16 aa, Artificial Sequence
  A synthetic peptide
VHYTSKDTLRKRHYWR

SEQ ID NO: 92 - protein, 14 aa, Artificial Sequence
  A synthetic peptide
DGQKRKKSLRKKLD

SEQ ID NO: 93 - protein, 15 aa, Artificial Sequence
  A synthetic peptide
AARKKRISVKKKQEQ

SEQ ID NO: 94 - protein, 15 aa, Artificial Sequence
  A synthetic peptide
KLAVGRHSFSRRSGV

SEQ ID NO: 95 - protein, 15 aa, Artificial Sequence
  A synthetic peptide
KKKFRTPSFLKKSKK

SEQ ID NO: 96 - protein, 18 aa, Artificial Sequence
  A synthetic peptide
RGFLRSASLGRRASFHLE

SEQ ID NO: 97 - protein, 17 aa, Artificial Sequence
  A synthetic peptide
KKRSSKKEASMKKVVRP

SEQ ID NO: 98 - protein, 15 aa, Artificial Sequence
  A synthetic peptide
AGPLRKSSLKKGGRL

SEQ ID NO: 99 - protein, 15 aa, Artificial Sequence
  A synthetic peptide
AGWRKKTSFRKPKED

SEQ ID NO: 100 - protein, 14 aa, Artificial Sequence
  A synthetic peptide
WKGKRRSKARKKRK

SEQ ID NO: 101 - protein, 13 aa, Artificial Sequence
  A synthetic peptide
GAPPRRSSIRNAH

SEQ ID NO: 102 - protein, 13 aa, Artificial Sequence
  A synthetic peptide
AGSFKRNSIKKIV

SEQ ID NO: 103 - protein, 17 aa, Artificial Sequence
  A synthetic peptide
LLKKRDSFRTPRDSKLE

SEQ ID NO: 104 - protein, 16 aa, Artificial Sequence
  A synthetic peptide
EKIKRSSLKKVDSLKK

SEQ ID NO: 105 - protein, 13 aa, Artificial Sequence
  A synthetic peptide
ALRRPSLRREADD

SEQ ID NO: 106 - protein, 15 aa, Artificial Sequence
  A synthetic peptide
EILSRRPSYRKILND

SEQ ID NO: 107 - protein, 15 aa, Artificial Sequence
  A synthetic peptide
QKRHARVTVKYDRRE

SEQ ID NO: 108 - protein, 15 aa, Artificial Sequence
  A synthetic peptide
KATTKKRTLRKNDRK

SEQ ID NO: 109 - protein, 15 aa, Artificial Sequence
  A synthetic peptide
KEVVRTDSLKGRRGR

SEQ ID NO: 110 - protein, 12 aa, Artificial Sequence
  A synthetic peptide
EGGDRRASGRRK

SEQ ID NO: 111 - protein, 20 aa, Artificial Sequence
  A synthetic peptide
KKKRFSFKKSFKLSGFSFKK

SEQ ID NO: 112 - protein, 15 aa, Artificial Sequence
  A synthetic peptide
KRPGKKGSNKRPGKR

SEQ ID NO: 113 - protein, 15 aa, Artificial Sequence
  A synthetic peptide
GENVLKKSMKSRVKG

SEQ ID NO: 114 - protein, 16 aa, Artificial Sequence
  A synthetic peptide
KKKKRASFKRKSSKKG

SEQ ID NO: 115 - protein, 13 aa, Artificial Sequence
  A synthetic peptide
NRKKKRTSFKRKA

SEQ ID NO: 116 - protein, 17 aa, Artificial Sequence
  A synthetic peptide
EEGTFRSSIRRLSTRRR

SEQ ID NO: 117 - protein, 13 aa, Artificial Sequence
  A synthetic peptide
NRKKKRTSFKRKA

SEQ ID NO: 118 - protein, 20 aa, Artificial Sequence
  A synthetic peptide
RPQNTLKASKKKKRASFKRK

SEQ ID NO: 119 - protein, 20 aa, Artificial Sequence
  A synthetic peptide
KKRFSFKKSFKLSGFSFKKN

SEQ ID NO: 120 - protein, 15 aa, Artificial Sequence
  A synthetic peptide
AKRRRLSSLRASTSK

SEQ ID NO: 121 - protein, 18 aa, Artificial Sequence
  A synthetic peptide
LRRRSLRRSNSISKSPGP

SEQ ID NO: 122 - protein, 15 aa, Artificial Sequence
  A synthetic peptide
RAITSTLASSFKRRR

SEQ ID NO: 123 - protein, 20 aa, Artificial Sequence
  A synthetic peptide
KKRFSFKKSFKLSGFSFKKN

SEQ ID NO: 124 - protein, 14 aa, Artificial Sequence
  A synthetic peptide
PRLIRRGSKKRPAR

SEQ ID NO: 125 - protein, 20 aa, Artificial Sequence
  A synthetic peptide
PLKEKKRERKTSSKSSVRKR

SEQ ID NO: 126 - protein, 20 aa, Artificial Sequence
  A synthetic peptide
KAIKAIEGGQKFARKSTRRS

SEQ ID NO: 127 - protein, 20 aa, Artificial Sequence
  A synthetic peptide
SQVQKQRSAGSFKRNSIKKI

SEQ ID NO: 128 - protein, 20 aa, Artificial Sequence
  A synthetic peptide
QQVDRERPHVRRRRGTFKRS

SEQ ID NO: 129 - protein, 20 aa, Artificial Sequence
  A synthetic peptide
VQRHRSMRKTFARYLSFRRD

SEQ ID NO: 130 - protein, 13 aa, Artificial Sequence
  A synthetic peptide
EYLERRASRRRAV

SEQ ID NO: 131 - protein, 14 aa, Artificial Sequence
  A synthetic peptide
WKGKRRSKARKKRK

SEQ ID NO: 132 - protein, 20 aa, Artificial Sequence
  A synthetic peptide
GFLNEPLSSKSQRRKSLKLK

SEQ ID NO: 133 - protein, 20 aa, Artificial Sequence
  A synthetic peptide
LEKRGMLGKRPRRKSSRRKK

SEQ ID NO: 134 - protein, 20 aa, Artificial Sequence
  A synthetic peptide
RSRSRSRSKSKDKRKSRKRS

SEQ ID NO: 135 - protein, 15 aa, Artificial Sequence
  A synthetic peptide
KKKFRTPSFLKKSKK

SEQ ID NO: 136 - protein, 13 aa, Artificial Sequence
  A synthetic peptide
RARRDSLKKIEIW

SEQ ID NO: 137 - protein, 20 aa, Artificial Sequence
  A synthetic peptide
PSKSPSKKKKKFRTPSFLKK

SEQ ID NO: 138 - protein, 13 aa, Artificial Sequence
  A synthetic peptide
EYLERRASRRRAV

SEQ ID NO: 139 - protein, 20 aa, Artificial Sequence
  A synthetic peptide
RPTPGDGEKRSRIKKSKKRK

SEQ ID NO: 140 - protein, 20 aa, Artificial Sequence
  A synthetic peptide
TELEGGFSRQRKRKLSFRRR

SEQ ID NO: 141 - protein, 20 aa, Artificial Sequence
  A synthetic peptide
VTDSQKRREILSRRPSYRKI

SEQ ID NO: 142 - protein, 20 aa, Artificial Sequence
  A synthetic peptide
ERHVAQKKSRLRRRASQLKI

SEQ ID NO: 143 - protein, 20 aa, Artificial Sequence
  A synthetic peptide
VRYTPYTISPYNRKGSFRKQ

SEQ ID NO: 144 - protein, 20 aa, Artificial Sequence
  A synthetic peptide
LSSMFGTLPRKSRKGSVRKQ

SEQ ID NO: 145 - protein, 20 aa, Artificial Sequence
  A synthetic peptide
ISDFGLAKKLAVGRHSFSRR

SEQ ID NO: 146 - protein, 20 aa, Artificial Sequence
  A synthetic peptide
QAQRQIKRGAPPRRSSIRNA

SEQ ID NO: 147 - protein, 20 aa, Artificial Sequence
  A synthetic peptide
RDIRQSPKRGFLRSASLGRR

SEQ ID NO: 148 - protein, 20 aa, Artificial Sequence
  A synthetic peptide
RELEQLKAEYLERRASRRRA

SEQ ID NO: 149 - protein, 18 aa, Artificial Sequence
  A synthetic peptide
RVVQSVKHTKRKSSTVMK

SEQ ID NO: 150 - protein, 20 aa, Artificial Sequence
  A synthetic peptide
VDPFYEMLAARKKRISVKKK

SEQ ID NO: 151 - protein, 20 aa, Artificial Sequence
  A synthetic peptide
PQNSLKASNRKKKRTSFKRK

SEQ ID NO: 152 - protein, 20 aa, Artificial Sequence
  A synthetic peptide
DLIEGRKGAQIVKRASLKRG

SEQ ID NO: 153 - protein, 20 aa, Artificial Sequence
  A synthetic peptide
TYLLPDKSRQGKRKTSIKRD

SEQ ID NO: 154 - protein, 20 aa, Artificial Sequence
  A synthetic peptide
KKFFTQGWAGWRKKTSFRKP

SEQ ID NO: 155 - protein, 20 aa, Artificial Sequence
  A synthetic peptide
RWDKRRWRKIPKRPGSVHRT

SEQ ID NO: 156 - protein, 20 aa, Artificial Sequence
  A synthetic peptide
SAQITIPKDGQKRKKSLRKK

SEQ ID NO: 157 - protein, 20 aa, Artificial Sequence
  A synthetic peptide
PSPSNETPKKKKKRFSFKKS

SEQ ID NO: 158 - protein, 20 aa, Artificial Sequence
  A synthetic peptide
VQMTWSYPDEKNKRASVRRR

SEQ ID NO: 159 - protein, 20 aa, Artificial Sequence
  A synthetic peptide
LYARLARAYRRSQRASFKRA

SEQ ID NO: 160 - protein, 20 aa, Artificial Sequence
  A synthetic peptide
PFEVVWYKDKRQLRSSKKYK

SEQ ID NO: 161 - protein, 20 aa, Artificial Sequence
  A synthetic peptide
KYKAFIRIPIPTRRHTFRRQ

SEQ ID NO: 162 - protein, 20 aa, Artificial Sequence
  A synthetic peptide
KKKFSFKKPFKLSGLSFKRN

SEQ ID NO: 163 - protein, 20 aa, Artificial Sequence
  A synthetic peptide
PPRTPGWHQLQPRRVSFRGE

SEQ ID NO: 164 - protein, 20 aa, Artificial Sequence [sequence omitted]

SEQ ID NO: 165 - protein, 20 aa, Artificial Sequence
  A synthetic peptide
TEEKSKKRKKKHRKNSRKHK

SEQ ID NO: 166 - protein, 20 aa, Artificial Sequence
  A synthetic peptide
MAQIERGEARIQRRISIKKA

SEQ ID NO: 167 - protein, 20 aa, Artificial Sequence
  A synthetic peptide
GLPAPGEDKSIYRRGSRRWR

SEQ ID NO: 168 - protein, 12 aa, Artificial Sequence
  A synthetic peptide
AMSRSASKRRSR

SEQ ID NO: 169 - protein, 11 aa, Artificial Sequence
  A synthetic peptide
RTRSRRLTFRK

SEQ ID NO: 170 - protein, 12 aa, Artificial Sequence
  A synthetic peptide
VKLRRSKKRTKR

SEQ ID NO: 171 - protein, 12 aa, Artificial Sequence
  A synthetic peptide
RRGRRSTKKRRR

SEQ ID NO: 172 - protein, 12 aa, Artificial Sequence
  A synthetic peptide
VRRRRSQRISQR

SEQ ID NO: 173 - protein, 11 aa, Artificial Sequence
  A synthetic peptide
RSGRRRGSQKS

SEQ ID NO: 174 - protein, 12 aa, Artificial Sequence
  A synthetic peptide
KKERRRNSINRN

SEQ ID NO: 175 - protein, 12 aa, Artificial Sequence
  A synthetic peptide
KKRRTKSSRRGV

SEQ ID NO: 176 - protein, 11 aa, Artificial Sequence
  A synthetic peptide
RRERSRSRRKQ

SEQ ID NO: 177 - protein, 12 aa, Artificial Sequence
  A synthetic peptide
RRRRRRSRTFSR

SEQ ID NO: 178 - protein, 11 aa, Artificial Sequence
  A synthetic peptide
RRRRSRTFSRS

SEQ ID NO: 179 - protein, 12 aa, Artificial Sequence
  A synthetic peptide
KRHYRKSVRSRS

SEQ ID NO: 180 - protein, 12 aa, Artificial Sequence
  A synthetic peptide
FLRRSSSRRNRS

SEQ ID NO: 181 - protein, 11 aa, Artificial Sequence
  A synthetic peptide
TGERKRKSVRG

SEQ ID NO: 182 - protein, 12 aa, Artificial Sequence
  A synthetic peptide
TKKKRGSYRGGS

SEQ ID NO: 183 - protein, 12 aa, Artificial Sequence
  A synthetic peptide
ARRSKRSRRRET

SEQ ID NO: 184 - protein, 10 aa, Artificial Sequence
  A synthetic peptide
FRASSRSTTK

SEQ ID NO: 185 - protein, 12 aa, Artificial Sequence
  A synthetic peptide
KKFKRRLSLTLR

SEQ ID NO: 186 - protein, 12 aa, Artificial Sequence
  A synthetic peptide
DFRRRRSFRRIA

SEQ ID NO: 187 - protein, 12 aa, Artificial Sequence
  A synthetic peptide
LRRKSSTRHIHA

SEQ ID NO: 188 - protein, 12 aa, Artificial Sequence
  A synthetic peptide
ERGRRGSKKGSI

SEQ ID NO: 189 - protein, 11 aa, Artificial Sequence
  A synthetic peptide
GRRRRSRSKVK

SEQ ID NO: 190 - protein, 12 aa, Artificial Sequence
  A synthetic peptide
RRRRHTMDKDSR

SEQ ID NO: 191 - protein, 11 aa, Artificial Sequence
  A synthetic peptide
HKRNSVRLVIR

SEQ ID NO: 192 - protein, 12 aa, Artificial Sequence
  A synthetic peptide
GNRKGKSKKWRQ

SEQ ID NO: 193 - protein, 12 aa, Artificial Sequence
  A synthetic peptide
PLRKSSLKKGGR

SEQ ID NO: 194 - protein, 12 aa, Artificial Sequence
  A synthetic peptide
KRRKRKSLQRHK

SEQ ID NO: 195 - protein, 10 aa, Artificial Sequence
  A synthetic peptide
PGSSHRKTKK

SEQ ID NO: 196 - protein, 12 aa, Artificial Sequence
  A synthetic peptide
RWKRRRSYSREH

SEQ ID NO: 197 - protein, 12 aa, Artificial Sequence
  A synthetic peptide
ILRPSKSVKLRS

SEQ ID NO: 198 - protein, 12 aa, Artificial Sequence
  A synthetic peptide
RRRRPTKSKGSK

SEQ ID NO: 199 - protein, 12 aa, Artificial Sequence
  A synthetic peptide
RGRRSRSRLRRR

SEQ ID NO: 200 - protein, 11 aa, Artificial Sequence
  A synthetic peptide
EQQRRALSFRQ

SEQ ID NO: 201 - protein, 12 aa, Artificial Sequence
  A synthetic peptide
TQDRRKSLFKKI

SEQ ID NO: 202 - protein, 12 aa, Artificial Sequence
  A synthetic peptide
VMKRKFSLRAAE

SEQ ID NO: 203 - protein, 12 aa, Artificial Sequence
  A synthetic peptide
VRRSKKSKKKES

SEQ ID NO: 204 - protein, 12 aa, Artificial Sequence
  A synthetic peptide
RFSRRSSSWRIL

SEQ ID NO: 205 - protein, 12 aa, Artificial Sequence
  A synthetic peptide
EGRRSRSRRYSG

SEQ ID NO: 206 - protein, 12 aa, Artificial Sequence
  A synthetic peptide
KSSRNSTSVKKK

SEQ ID NO: 207 - protein, 12 aa, Artificial Sequence
  A synthetic peptide
SFRGHITRKKLK

SEQ ID NO: 208 - protein, 12 aa, Artificial Sequence
  A synthetic peptide
VSRPRKSRKRVD

SEQ ID NO: 209 - protein, 12 aa, Artificial Sequence
  A synthetic peptide
DKEKSKGSLKRK

SEQ ID NO: 210 - protein, 12 aa, Artificial Sequence
  A synthetic peptide
PLRRRESMHVEQ

SEQ ID NO: 211 - protein, 11 aa, Artificial Sequence
  A synthetic peptide
RSRSYSRSRSR

SEQ ID NO: 212 - protein, 12 aa, Artificial Sequence
  A synthetic peptide
VSRGSSLKILSK

SEQ ID NO: 213 - protein, 12 aa, Artificial Sequence
  A synthetic peptide
RHSRSRSRHRLS

SEQ ID NO: 214 - protein, 12 aa, Artificial Sequence
  A synthetic peptide
SRRRSPSYSRHS

SEQ ID NO: 215 - protein, 12 aa, Artificial Sequence
  A synthetic peptide
TKKRSKSRSKER

SEQ ID NO: 216 - protein, 12 aa, Artificial Sequence
  A synthetic peptide
SCRTSSRKRAGK

SEQ ID NO: 217-228 - skipped (12 entries)

SEQ ID NO: 229 - protein, 7 aa, Artificial Sequence
  A synthetic peptide
  SITE 4: Xaa = pSer (phosphorylated serine)
WKNXIRH

SEQ ID NO: 230 - protein, 7 aa, Artificial Sequence
  A synthetic peptide
  SITE 4: Xaa = pSer (phosphorylated serine)
RRPXYRK

SEQ ID NO: 231 - protein, 5 aa, Artificial Sequence
  A synthetic peptide
  SITE 1: Xaa = biotinylated-Lys
  SITE 2: Xaa = dansylated-Lys
XXPPG

SEQ ID NO: 232-288 - skipped (57 entries)

SEQ ID NO: 289 - protein, 17 aa, Artificial Sequence
  A synthetic peptide
  SITE 9: Xaa = pS (phosphorylated serine)
MKNAHAKAXRTSSKHKE

SEQ ID NO: 290 - protein, 17 aa, Artificial Sequence
  A synthetic peptide
  SITE 9: Xaa = pS (phosphorylated serine)
RVILQGRDXNIPGSDYI

SEQ ID NO: 291 - protein, 17 aa, Artificial Sequence
  A synthetic peptide
  SITE 9: Xaa = pS (phosphorylated serine)
AHAKASRTXSKHKEDVY

SEQ ID NO: 292 - protein, 17 aa, Artificial Sequence
  A synthetic peptide
  SITE 9: Xaa = pS (phosphorylated serine)
KKKLEVLQXQKGQESEY

SEQ ID NO: 293 - protein, 17 aa, Artificial Sequence
  A synthetic peptide
  SITE 9: Xaa = pS (phosphorylated serine)
PSEPGGVLXFLDQINQR

SEQ ID NO: 294 - protein, 17 aa, Artificial Sequence
  A synthetic peptide
  SITE 9: Xaa = pS (phosphorylated serine)
HAKASRTSXKHKEDVYE

SEQ ID NO: 295 - protein, 17 aa, Artificial Sequence
  A synthetic peptide
  SITE 9: Xaa = pS (phosphorylated serine)
PWTFLVREXLSQPGDFV

SEQ ID NO: 296 - protein, 17 aa, Artificial Sequence
  A synthetic peptide
  SITE 9: Xaa = pS (phosphorylated serine)
KNQGDFSLXVRVGDQVT

SEQ ID NO: 297 - protein, 17 aa, Artificial Sequence
  A synthetic peptide
  SITE 9: Xaa = pS (phosphorylated serine)
PLNCSDPTXERWYHGHM

SEQ ID NO: 298 - protein, 13 aa, Artificial Sequence
  A synthetic peptide
  SITE 9: Xaa = pS (phosphorylated serine)
ADKEKSKGXLKRK

SEQ ID NO: 299 - protein, 17 aa, Artificial Sequence
  A synthetic peptide
  SITE 9: Xaa = pS (phosphorylated serine)
LKGRGVHGXFLARPSRK

SEQ ID NO: 300 - protein, 17 aa, Artificial Sequence
  A synthetic peptide
  SITE 9: Xaa = pS (phosphorylated serine)
HGSFLARPXRKNQGDFS

SEQ ID NO: 301 - protein, 14 aa, Artificial Sequence
  A synthetic peptide
  SITE 8: Xaa = pS (phosphorylated serine)
RSGRRRGXQKSTDS

SEQ ID NO: 302 - protein, 14 aa, Artificial Sequence
  A synthetic peptide
  SITE 8: Xaa = pS (phosphorylated serine)
AQIVKRAXLKRGKQ

SEQ ID NO: 303 - protein, 14 aa, Artificial Sequence
  A synthetic peptide
  SITE 8: Xaa = pS (phosphorylated serine)
FERGRRGXKKGSID

SEQ ID NO: 304 - protein, 14 aa, Artificial Sequence
  A synthetic peptide
  SITE 8: Xaa = pS (phosphorylated serine)
SEGRRSRXRRYSGS

SEQ ID NO: 305 - protein, 13 aa, Artificial Sequence
  A synthetic peptide
  SITE 8: Xaa = pS (phosphorylated serine)
FKRRKRKXLQRHK

SEQ ID NO: 306 - protein, 14 aa, Artificial Sequence
  A synthetic peptide
  SITE 8: Xaa = pS (phosphorylated serine)
IKKRRTKXSRRGVS

SEQ ID NO: 307 - protein, 14 aa, Artificial Sequence
  A synthetic peptide
  SITE 8: Xaa = pS (phosphorylated serine)
KKRRTKSXRRGVSR

SEQ ID NO: 308 - protein, 14 aa, Artificial Sequence
  A synthetic peptide
  SITE 8: Xaa = pS (phosphorylated serine)
PEVKLRRXKKRTKR

SEQ ID NO: 309 - protein, 14 aa, Artificial Sequence
  A synthetic peptide
  SITE 8: Xaa = pS (phosphorylated serine)
YSLVRTRXRRLTFR

SEQ ID NO: 310 - protein, 14 aa, Artificial Sequence
  A synthetic peptide
  SITE 8: Xaa = pS (phosphorylated serine)
GGNRKGKXKKWRQM

SEQ ID NO: 311 - protein, 11 aa, Artificial Sequence
  A synthetic peptide
  SITE 5: Xaa = pS (phosphorylated serine)
LRRRXLRRSNS

SEQ ID NO: 312 - protein, 14 aa, Artificial Sequence
  A synthetic peptide
  SITE 8: Xaa = pS (phosphorylated serine)
RFSRRSSXWRILGS

SEQ ID NO: 313 - protein, 14 aa, Artificial Sequence
  A synthetic peptide
  SITE 8: Xaa = pS (phosphorylated serine)
RRGTFKRXKLRARD

SEQ ID NO: 314 - protein, 14 aa, Artificial Sequence
  A synthetic peptide
  SITE 8: Xaa = pS (phosphorylated serine)
HVRRRRGXFKRSKL

SEQ ID NO: 315 - protein, 14 aa, Artificial Sequence
  A synthetic peptide
  SITE 8: Xaa = pS (phosphorylated serine)
KKERRRNXINRNFV

SEQ ID NO: 316 - protein, 14 aa, Artificial Sequence
  A synthetic peptide
  SITE 8: Xaa = pS (phosphorylated serine)
TSSYRSRXYSRSRS

SEQ ID NO: 317 - protein, 14 aa, Artificial Sequence
  A synthetic peptide
  SITE 8: Xaa = pS (phosphorylated serine)
KKFKRRLXLTLRGS

SEQ ID NO: 318 - protein, 13 aa, Artificial Sequence
  A synthetic peptide
  SITE 8: Xaa = pS (phosphorylated serine)
RKTSSKSXVRKRR

SEQ ID NO: 319 - protein, 14 aa, Artificial Sequence
  A synthetic peptide
  SITE 8: Xaa = pS (phosphorylated serine)
SDFRRRRXFRRIAG

SEQ ID NO: 320 - protein, 12 aa, Artificial Sequence
  A synthetic peptide
  SITE 8: Xaa = pS (phosphorylated serine)
DKEKSKGXLKRK

SEQ ID NO: 321 - protein, 12 aa, Artificial Sequence
  A synthetic peptide
  SITE 8: Xaa = pT (phosphorylated threonine)
RALSFRQXSWLS

SEQ ID NO: 322 - protein, 14 aa, Artificial Sequence
  A synthetic peptide
  SITE 8: Xaa = pS (phosphorylated serine)
EQQRRALXFRQTSW

SEQ ID NO: 323 - protein, 14 aa, Artificial Sequence
  A synthetic peptide
  SITE 8: Xaa = pS (phosphorylated serine)
QAMSRSAXKRRSRF

SEQ ID NO: 324 - protein, 14 aa, Artificial Sequence
  A synthetic peptide
  SITE 8: Xaa = pS (phosphorylated serine)
KTKKKRGXYRGGSI

SEQ ID NO: 325 - protein, 14 aa, Artificial Sequence
  A synthetic peptide
  SITE 8: Xaa = pS (phosphorylated serine)
AARKKRIXVKKKQE

SEQ ID NO: 326 - protein, 12 aa, Artificial Sequence
  A synthetic peptide
  SITE 8: Xaa = pS (phosphorylated serine)
KKKHRKNXRKHK

SEQ ID NO: 327 - protein, 14 aa, Artificial Sequence
  A synthetic peptide
  SITE 8: Xaa = pS (phosphorylated serine)
FGTLPRKXRKGSVR

SEQ ID NO: 328 - protein, 12 aa, Artificial Sequence
  A synthetic peptide
  SITE 8: Xaa = pS (phosphorylated serine)
PRKSRKGXVRKQ

SEQ ID NO: 329 - protein, 11 aa, Artificial Sequence
  A synthetic peptide
  SITE 8: Xaa = pS (phosphorylated serine)
SSSRRNRXISN

SEQ ID NO: 330 - protein, 14 aa, Artificial Sequence
  A synthetic peptide
  SITE 8: Xaa = pS (phosphorylated serine)
DFLRRSSXRRNRSI

SEQ ID NO: 331 - protein, 12 aa, Artificial Sequence
  A synthetic peptide
  SITE 6: Xaa = pS (phosphorylated serine)
RMARRXKRSRRR

SEQ ID NO: 332 - protein, 14 aa, Artificial Sequence
  A synthetic peptide
  SITE 8: Xaa = pS (phosphorylated serine)
ETQDRRKXLFKKIS

SEQ ID NO: 333 - protein, 14 aa, Artificial Sequence
  A synthetic peptide
  SITE 8: Xaa = pS (phosphorylated serine)
MARRSKRXRRRETQ

SEQ ID NO: 334 - protein, 12 aa, Artificial Sequence
  A synthetic peptide
  SITE 8: Xaa = pS (phosphorylated serine)
RRRSQRIXQRIT

SEQ ID NO: 335 - protein, 14 aa, Artificial Sequence
  A synthetic peptide
  SITE 8: Xaa = pS (phosphorylated serine)
SGVRRRRXQRISQR

SEQ ID NO: 336 - protein, 14 aa, Artificial Sequence
  A synthetic peptide
  SITE 8: Xaa = pS (phosphorylated serine)
VILRPSKXVKLRSP

SEQ ID NO: 337 - protein, 14 aa, Artificial Sequence
  A synthetic peptide
  SITE 8: Xaa = pS (phosphorylated serine)
RRRRPTKXKGSKSS

SEQ ID NO: 338 - protein, 14 aa, Artificial Sequence
  A synthetic peptide
  SITE 8: Xaa = pT (phosphorylated threonine)
SGRRRRPXKSKGSK

SEQ ID NO: 339 - protein, 11 aa, Artificial Sequence
  A synthetic peptide
  SITE 8: Xaa = pS (phosphorylated serine)
RKSVRSRXRHE

SEQ ID NO: 340 - protein, 14 aa, Artificial Sequence
  A synthetic peptide
  SITE 8: Xaa = pS (phosphorylated serine)
TKRHYRKXVRSRSR

SEQ ID NO: 341 - protein, 14 aa, Artificial Sequence
  A synthetic peptide
  SITE 8: Xaa = pS (phosphorylated serine)
AGPLRKSXLKKGGR

SEQ ID NO: 342 - protein, 14 aa, Artificial Sequence
  A synthetic peptide
  SITE 8: Xaa = pS (phosphorylated serine)
EKSHKRNXVRLVIR

SEQ ID NO: 343 - protein, 10 aa, Artificial Sequence
  A synthetic peptide
  SITE 8: Xaa = pS (phosphorylated serine)
TPSFLKKXKK

SEQ ID NO: 344 - protein, 8 aa, Artificial Sequence
  A synthetic peptide
  SITE 3: Xaa = pS (phosphorylated serine)
RRXGRRRG

SEQ ID NO: 345 - protein, 14 aa, Artificial Sequence
  A synthetic peptide
  SITE 8: Xaa = pS (phosphorylated serine)
DGQKRKKXLRKKLD

SEQ ID NO: 346 - protein, 14 aa, Artificial Sequence
  A synthetic peptide
  SITE 8: Xaa = pS (phosphorylated serine)
NRLRRKSXTRHIHA

SEQ ID NO: 347 - protein, 13 aa, Artificial Sequence
  A synthetic peptide
  SITE 7: Xaa = pS (phosphorylated serine)
NRLRRKXSTRHIH

SEQ ID NO: 348 - protein, 12 aa, Artificial Sequence
  A synthetic peptide
  SITE 8: Xaa = pS (phosphorylated serine)
RGFLRSAXLGRR

SEQ ID NO: 349 - protein, 13 aa, Artificial Sequence
  A synthetic peptide
  SITE 7: Xaa = pS (phosphorylated serine)
GSEGRRXRSRRYS

SEQ ID NO: 350 - protein, 14 aa, Artificial Sequence
  A synthetic peptide
  SITE 8: Xaa = pT (phosphorylated threonine)
RRRRRSRXFSRSSS

SEQ ID NO: 351 - protein, 12 aa, Artificial Sequence
  A synthetic peptide
  SITE 8: Xaa = pS (phosphorylated serine)
RRRSRTFXRSSS

SEQ ID NO: 352 - protein, 14 aa, Artificial Sequence
  A synthetic peptide
  SITE 8: Xaa = pS (phosphorylated serine)
YRWKRRRXYSREHE

SEQ ID NO: 353 - protein, 12 aa, Artificial Sequence
  A synthetic peptide
  SITE 8: Xaa = pT (phosphorylated threonine)
LRRSKKRXKRSS

SEQ ID NO: 354 - protein, 14 aa, Artificial Sequence
  A synthetic peptide
  SITE 8: Xaa = pS (phosphorylated serine)
KLAVGRHXFSRRSG

SEQ ID NO: 355 - protein, 10 aa, Artificial Sequence
  A synthetic peptide
  SITE 8: Xaa = pS (phosphorylated serine)
AVGRHSFXRR

SEQ ID NO: 356 - protein, 13 aa, Artificial Sequence
  A synthetic peptide
  SITE 7: Xaa = pS (phosphorylated serine)
RERRERXRSRRKQ

SEQ ID NO: 357 - protein, 14 aa, Artificial Sequence
  A synthetic peptide
  SITE 8: Xaa = pS (phosphorylated serine)
ERRERSRXRRKQHL

SEQ ID NO: 358 - protein, 12 aa, Artificial Sequence
  A synthetic peptide
  SITE 8: Xaa = pS (phosphorylated serine)
KRPRRKSXRRKK

SEQ ID NO: 359 - protein, 13 aa, Artificial Sequence
  A synthetic peptide
  SITE 8: Xaa = pS (phosphorylated serine)
GKRPRRKXSRRKK

SEQ ID NO: 360 - protein, 14 aa, Artificial Sequence
  A synthetic peptide
  SITE 8: Xaa = pT (phosphorylated threonine)
KATTKKRXLRKNDR

SEQ ID NO: 361 - protein, 14 aa, Artificial Sequence
  A synthetic peptide
  SITE 8: Xaa = pS (phosphorylated serine)
RRRSLRRXNSISKS

SEQ ID NO: 362 - protein, 14 aa, Artificial Sequence
  A synthetic peptide
  SITE 8: Xaa = pS (phosphorylated serine)
RSLRRSNXISKSPG

SEQ ID NO: 363 - protein, 14 aa, Artificial Sequence
  A synthetic peptide
  SITE 8: Xaa = pS (phosphorylated serine)
DRFSRRSXSWRILG

SEQ ID NO: 364 - protein, 13 aa, Artificial Sequence
  A synthetic peptide
  SITE 7: Xaa = pS (phosphorylated serine)
DRFSRRXSSWRIL

SEQ ID NO: 365 - protein, 12 aa, Artificial Sequence
  A synthetic peptide
  SITE 6: Xaa = pS (phosphorylated serine)
VQRHRXMRKTFA

SEQ ID NO: 366 - protein, 13 aa, Artificial Sequence
  A synthetic peptide
  SITE 8: Xaa = pS (phosphorylated serine)
AGSFKRNXIKKIV

SEQ ID NO: 367 - protein, 14 aa, Artificial Sequence
  A synthetic peptide
  SITE 8: Xaa = pS (phosphorylated serine)
SYRSRSYXRSRSRG

SEQ ID NO: 368 - protein, 12 aa, Artificial Sequence
  A synthetic peptide
  SITE 8: Xaa = pS (phosphorylated serine)
RSRSYSRXRSRG

SEQ ID NO: 369 - protein, 11 aa, Artificial Sequence
  A synthetic peptide
  SITE 8: Xaa = pT (phosphorylated threonine)
RASSRSTXKKR

SEQ ID NO: 370 - protein, 12 aa, Artificial Sequence
  A synthetic peptide
  SITE 8: Xaa = pT (phosphorylated threonine)
FRASSRSXTKKR

SEQ ID NO: 371 - protein, 14 aa, Artificial Sequence
  A synthetic peptide
  SITE 8: Xaa = pT (phosphorylated threonine)
FKRRLSLXLRGSQT

SEQ ID NO: 372 - protein, 12 aa, Artificial Sequence
  A synthetic peptide
  SITE 6: Xaa = pT (phosphorylated threonine)
KRERKXSSKSSV

SEQ ID NO: 373 - protein, 14 aa, Artificial Sequence
  A synthetic peptide
  SITE 8: Xaa = pS (phosphorylated serine)
KKRERKTXSKSSVR

SEQ ID NO: 374 - protein, 12 aa, Artificial Sequence
  A synthetic peptide
  SITE 7: Xaa = pS (phosphorylated serine)
KHTKRKSXTVMK

SEQ ID NO: 375 - protein, 14 aa, Artificial Sequence
  A synthetic peptide
  SITE 8: Xaa = pT (phosphorylated threonine)
VHYTSKDXLRKRHY

SEQ ID NO: 376 - protein, 12 aa, Artificial Sequence
  A synthetic peptide
  SITE 8: Xaa = pS (phosphorylated serine)
KSIYRRGXRRWR

SEQ ID NO: 377 - protein, 14 aa, Artificial Sequence
  A synthetic peptide
  SITE 8: Xaa = pS (phosphorylated serine)
NVMKRKFXLRAAEF

SEQ ID NO: 378 - protein, 12 aa, Artificial Sequence
  A synthetic peptide
  SITE 8: Xaa = pS (phosphorylated serine)
RSASKRRXRFSS

SEQ ID NO: 379 - protein, 13 aa, Artificial Sequence
  A synthetic peptide
  SITE 7: Xaa = pS (phosphorylated serine)
EKIKRSXLKKVDS

SEQ ID NO: 380 - protein, 14 aa, Artificial Sequence
  A synthetic peptide
  SITE 8: Xaa = pS (phosphorylated serine)
EISCRTSXRKRAGK

SEQ ID NO: 381 - protein, 13 aa, Artificial Sequence
  A synthetic peptide
  SITE 7: Xaa = pS (phosphorylated serine)
WKGKRRXKARKKR

SEQ ID NO: 382 - protein, 12 aa, Artificial Sequence
  A synthetic peptide
  SITE 6: Xaa = pS (phosphorylated serine)
RARRDXLKKIEI

SEQ ID NO: 383 - protein, 12 aa, Artificial Sequence
  A synthetic peptide
  SITE 6: Xaa = pS (phosphorylated serine)
ALRRPXLRREAD

SEQ ID NO: 384 - protein, 13 aa, Artificial Sequence
  A synthetic peptide
  SITE 7: Xaa = pT (phosphorylated threonine)
NRKKKRXSFKRKA

SEQ ID NO: 385 - protein, 14 aa, Artificial Sequence
  A synthetic peptide
  SITE 8: Xaa = pS (phosphorylated serine)
DDFLRRSXSRRNRS

SEQ ID NO: 386 - protein, 14 aa, Artificial Sequence
  A synthetic peptide
  SITE 8: Xaa = pS (phosphorylated serine)
PRLIRRGXKKRPAR

SEQ ID NO: 387 - protein, 12 aa, Artificial Sequence
  A synthetic peptide
  SITE 8: Xaa = pS (phosphorylated serine)
MILKRRKXLKQK

SEQ ID NO: 388 - protein, 14 aa, Artificial Sequence
  A synthetic peptide
  SITE 8: Xaa = pS (phosphorylated serine)
KRPGKKGXNKRPGK

SEQ ID NO: 389 - protein, 14 aa, Artificial Sequence
  A synthetic peptide
  SITE 8: Xaa = pS (phosphorylated serine)
MVRRSKKXKKKESL

SEQ ID NO: 390 - protein, 12 aa, Artificial Sequence
  A synthetic peptide
  SITE 6: Xaa = pS (phosphorylated serine)
RMVRRXKKSKKK

SEQ ID NO: 391 - protein, 14 aa, Artificial Sequence
  A synthetic peptide
  SITE 8: Xaa = pS (phosphorylated serine)
EVSRPRKXRKRVDS

SEQ ID NO: 392 - protein, 14 aa, Artificial Sequence
  A synthetic peptide
  SITE 8: Xaa = pS (phosphorylated serine)
ARIIGEKXFRRSVV

SEQ ID NO: 393 - protein, 13 aa, Artificial Sequence
  A synthetic peptide
  SITE 7: Xaa = pS (phosphorylated serine)
SVILRPXKSVKLR

SEQ ID NO: 394 - protein, 13 aa, Artificial Sequence
  A synthetic peptide
  SITE 8: Xaa = pS (phosphorylated serine)
EYLERRAXRRRAV

SEQ ID NO: 395 - protein, 14 aa, Artificial Sequence
  A synthetic peptide
  SITE 8: Xaa = pS (phosphorylated serine)
KKRRGRRXTKKRRR

SEQ ID NO: 396 - protein, 14 aa, Artificial Sequence
  A synthetic peptide
  SITE 8: Xaa = pT (phosphorylated threonine)
KRRGRRSXKKRRRR

SEQ ID NO: 397 - protein, 12 aa, Artificial Sequence
  A synthetic peptide
  SITE 8: Xaa = pS (phosphorylated serine)
SKSQRRKXLKLK

SEQ ID NO: 398 - protein, 12 aa, Artificial Sequence
  A synthetic peptide
  SITE 8: Xaa = pS (phosphorylated serine)
EGGDRRAXGRRK

SEQ ID NO: 399 - protein, 14 aa, Artificial Sequence
  A synthetic peptide
  SITE 8: Xaa = pT (phosphorylated threonine)
EYRRRRHXMDKDSR

SEQ ID NO: 400 - protein, 14 aa, Artificial Sequence
  A synthetic peptide
  SITE 8: Xaa = pT (phosphorylated threonine)
RLRRKSSXRHIHAL

SEQ ID NO: 401 - protein, 14 aa, Artificial Sequence
  A synthetic peptide
  SITE 8: Xaa = pS (phosphorylated serine)
KIPKRPGXVHRTPS

SEQ ID NO: 402 - protein, 13 aa, Artificial Sequence
  A synthetic peptide
  SITE 7: Xaa = pS (phosphorylated serine)
RRRRRRXRTFSRS

SEQ ID NO: 403 - protein, 12 aa, Artificial Sequence
  A synthetic peptide
  SITE 8: Xaa = pS (phosphorylated serine)
RSRTFSRXSSMK

SEQ ID NO: 404 - protein, 10 aa, Artificial Sequence
  A synthetic peptide
  SITE 8: Xaa = pS (phosphorylated serine)
RTFSRSSXMK

SEQ ID NO: 405 - protein, 7 aa, Artificial Sequence
  A synthetic peptide
  SITE 1: Xaa = pS (phosphorylated serine)
XYRWKRR

SEQ ID NO: 406 - protein, 14 aa, Artificial Sequence
  A synthetic peptide
  SITE 8: Xaa = pS (phosphorylated serine)
WKRRRSYXREHEGR

SEQ ID NO: 407 - protein, 13 aa, Artificial Sequence
  A synthetic peptide
  SITE 7: Xaa = pT (phosphorylated threonine)
FIKKRRXKSSRRG

SEQ ID NO: 408 - protein, 10 aa, Artificial Sequence
  A synthetic peptide
  SITE 8: Xaa = pS (phosphorylated serine)
KSSRRGVXRE

SEQ ID NO: 409 - protein, 9 aa, Artificial Sequence
  A synthetic peptide
  SITE 8: Xaa = pS (phosphorylated serine)
SKKRTKRXS

SEQ ID NO: 410 - protein, 10 aa, Artificial Sequence
  A synthetic peptide
  SITE 8: Xaa = pS (phosphorylated serine)
RHSFSRRXGV

SEQ ID NO: 411 - protein, 14 aa, Artificial Sequence
  A synthetic peptide
  SITE 8: Xaa = pS (phosphorylated serine)
ASFRGHIXRKKLKG

SEQ ID NO: 412 - protein, 12 aa, Artificial Sequence
  A synthetic peptide
  SITE 8: Xaa = pS (phosphorylated serine)
YRRSQRAXFKRA

SEQ ID NO: 413 - protein, 14 aa, Artificial Sequence
  A synthetic peptide
  SITE 8: Xaa = pS (phosphorylated serine)
LARAYRRXQRASFK

SEQ ID NO: 414 - protein, 9 aa, Artificial Sequence
  A synthetic peptide
  SITE 3: Xaa = pT (phosphorylated threonine)
KAXTKKRTL

SEQ ID NO: 415 - protein, 10 aa, Artificial Sequence
  A synthetic peptide
  SITE 4: Xaa = pS (phosphorylated serine)
KATXKKRTLR

SEQ ID NO: 416 - protein, 14 aa, Artificial Sequence
  A synthetic peptide
  SITE 8: Xaa = pS (phosphorylated serine)
RHRSMRKXFARYLS

SEQ ID NO: 417 - protein, 12 aa, Artificial Sequence
  A synthetic peptide
  SITE 8: Xaa = pS (phosphorylated serine)
KTFARYLXFRRD

SEQ ID NO: 418 - protein, 14 aa, Artificial Sequence
  A synthetic peptide
  SITE 8: Xaa = pS (phosphorylated serine)
QKQRSAGXFKRNSI

SEQ ID NO: 419 - protein, 10 aa, Artificial Sequence
  A synthetic peptide
  SITE 8: Xaa = pS (phosphorylated serine)
RSYSRSRXRG

SEQ ID NO: 420 - protein, 14 aa, Artificial Sequence
  A synthetic peptide
  SITE 8: Xaa = pS (phosphorylated serine)
NTQQFRAXSRSTTK

SEQ ID NO: 421 - protein, 14 aa, Artificial Sequence
  A synthetic peptide
  SITE 8: Xaa = pS (phosphorylated serine)
TQQFRASXRSTTKK

SEQ ID NO: 422 - protein, 13 aa, Artificial Sequence
  A synthetic peptide
  SITE 8: Xaa = pS (phosphorylated serine)
VKHTKRKXSTVMK

SEQ ID NO: 423 - protein, 11 aa, Artificial Sequence
  A synthetic peptide
  SITE 8: Xaa = pT (phosphorylated threonine)
HTKRKSSXVMK

SEQ ID NO: 424 - protein, 11 aa, Artificial Sequence
  A synthetic peptide
  SITE 5: Xaa = pS (phosphorylated serine)
RVVQXVKHTKR

SEQ ID NO: 425 - protein, 12 aa, Artificial Sequence
  A synthetic peptide
  SITE 8: Xaa = pS (phosphorylated serine)
KSKDKRKXRKRS

SEQ ID NO: 426 - protein, 8 aa, Artificial Sequence
  A synthetic peptide
  SITE 8: Xaa = pS (phosphorylated serine)
KRKSRKRX

SEQ ID NO: 427 - protein, 14 aa, Artificial Sequence
  A synthetic peptide
  SITE 8: Xaa = pS (phosphorylated serine)
RSRSRSKXKDKRKS

SEQ ID NO: 428 - protein, 14 aa, Artificial Sequence
  A synthetic peptide
  SITE 8: Xaa = pS (phosphorylated serine)
RSRSRSRXKSKDKR

SEQ ID NO: 429 - protein, 10 aa, Artificial Sequence
  A synthetic peptide
  SITE 4: Xaa = pS (phosphorylated serine)
RSRXRSRSKS

SEQ ID NO: 430 - protein, 12 aa, Artificial Sequence
  A synthetic peptide
  SITE 6: Xaa = pS (phosphorylated serine)
RSRSRXRSKSKD

SEQ ID NO: 431 - protein, 12 aa, Artificial Sequence
  A synthetic peptide
  SITE 8: Xaa = pS (phosphorylated serine)
ARIQRRIXIKKA

SEQ ID NO: 432 - protein, 14 aa, Artificial Sequence
  A synthetic peptide
  SITE 8: Xaa = pS (phosphorylated serine)
APLRRREXMHVEQR

SEQ ID NO: 433 - protein, 12 aa, Artificial Sequence
  A synthetic peptide
  SITE 8: Xaa = pS (phosphorylated serine)
DKKQIRSXKKYR

SEQ ID NO: 434 - protein, 13 aa, Artificial Sequence
  A synthetic peptide
  SITE 8: Xaa = pS (phosphorylated serine)
KDKRQLRXSKKYK

SEQ ID NO: 435 - protein, 11 aa, Artificial Sequence
  A synthetic peptide
  SITE 8: Xaa = pS (phosphorylated serine)
SSLKKVDXLKK

SEQ ID NO: 436 - protein, 12 aa, Artificial Sequence
  A synthetic peptide
  SITE 8: Xaa = pS (phosphorylated serine)
SVRQRRMXLRDD

SEQ ID NO: 437 - protein, 11 aa, Artificial Sequence
  A synthetic peptide
  SITE 8: Xaa = pS (phosphorylated serine)
SRSRHRLXRSR

SEQ ID NO: 438 - protein, 13 aa, Artificial Sequence
  A synthetic peptide
  SITE 7: Xaa = pS (phosphorylated serine)
SSRHSRXRSRHRL

SEQ ID NO: 439 - protein, 14 aa, Artificial Sequence
  A synthetic peptide
  SITE 8: Xaa = pS (phosphorylated serine)
YSRRRSPXYSRHSS

SEQ ID NO: 440 - protein, 14 aa, Artificial Sequence
  A synthetic peptide
  SITE 8: Xaa = pS (phosphorylated serine)
SRHSRSRXRHRLSR

SEQ ID NO: 441 - protein, 13 aa, Artificial Sequence
  A synthetic peptide
  SITE 7: Xaa = pS (phosphorylated serine)
RDRGRRXRSRLRR

SEQ ID NO: 442 - protein, 10 aa, Artificial Sequence
  A synthetic peptide
  SITE 8: Xaa = pS (phosphorylated serine)
RSRLRRRXRS

SEQ ID NO: 443 - protein, 14 aa, Artificial Sequence
  A synthetic peptide
  SITE 8: Xaa = pS (phosphorylated serine)
RGGRRRRXRSKVKE

SEQ ID NO: 444 - protein, 14 aa, Artificial Sequence
  A synthetic peptide
  SITE 8: Xaa = pS (phosphorylated serine)
TTKKRSKXRSKERT

SEQ ID NO: 445 - protein, 14 aa, Artificial Sequence
  A synthetic peptide
  SITE 8: Xaa = pS (phosphorylated serine)
DRGRRSRXRLRRRS

SEQ ID NO: 446 - protein, 8 aa, Artificial Sequence
  A synthetic peptide
  SITE 8: Xaa = pS (phosphorylated serine)
RLRRRSRX

SEQ ID NO: 447 - protein, 14 aa, Artificial Sequence
  A synthetic peptide
  SITE 8: Xaa = pS (phosphorylated serine)
GRRRRSRXKVKEDK

SEQ ID NO: 448 - protein, 13 aa, Artificial Sequence
  A synthetic peptide
  SITE 7: Xaa = pS (phosphorylated serine)
KKRKKKXSKSLAH

SEQ ID NO: 449 - protein, 14 aa, Artificial Sequence
  A synthetic peptide
  SITE 8: Xaa = pS (phosphorylated serine)
KKRKKKSXKSLAHA

SEQ ID NO: 450 - protein, 12 aa, Artificial Sequence
  A synthetic peptide
  SITE 8: Xaa = pS (phosphorylated serine)
QLQPRRVXFRGE

SEQ ID NO: 451 - protein, 9 aa, Artificial Sequence
  A synthetic peptide
  SITE 3: Xaa = pT (phosphorylated threonine)
EKXKKKRGS

SEQ ID NO: 452 - protein, 11 aa, Artificial Sequence
  A synthetic peptide
  SITE 8: Xaa = pS (phosphorylated serine)
RGSYRGGXISV

SEQ ID NO: 453 - protein, 11 aa, Artificial Sequence
  A synthetic peptide
  SITE 5: Xaa = pS (phosphorylated serine)
TEEKXKKRKKK

SEQ ID NO: 454 - protein, 12 aa, Artificial Sequence
  A synthetic peptide
  SITE 8: Xaa = pS (phosphorylated serine)
AGWRKKTXFRKP

SEQ ID NO: 455 - protein, 13 aa, Artificial Sequence
  A synthetic peptide
  SITE 7: Xaa = pT (phosphorylated threonine)
AGWRKKXSFRKPK

SEQ ID NO: 456 - protein, 13 aa, Artificial Sequence
  A synthetic peptide
  SITE 7: Xaa = pS (phosphorylated serine)
DDFLRRXSSRRNR

SEQ ID NO: 457 - protein, 11 aa, Artificial Sequence
  A synthetic peptide
  SITE 8: Xaa = pS (phosphorylated serine)
STSVKKKXSRN

SEQ ID NO: 458 - protein, 10 aa, Artificial Sequence
  A synthetic peptide
  SITE 8: Xaa = pS (phosphorylated serine)
TSVKKKSXRN

SEQ ID NO: 459 - protein, 14 aa, Artificial Sequence
  A synthetic peptide
  SITE 8: Xaa = pS (phosphorylated serine)
KSSRNSTXVKKKSS

SEQ ID NO: 460 - protein, 14 aa, Artificial Sequence
  A synthetic peptide
  SITE 8: Xaa = pT (phosphorylated threonine)
LKSSRNSXSVKKKS

SEQ ID NO: 461 - protein, 13 aa, Artificial Sequence
  A synthetic peptide
  SITE 7: Xaa = pS (phosphorylated serine)
LKSSRNXTSVKKK

SEQ ID NO: 462 - protein, 11 aa, Artificial Sequence
  A synthetic peptide
  SITE 8: Xaa = pT (phosphorylated threonine)
KRSRRREXQDR

SEQ ID NO: 463 - protein, 8 aa, Artificial Sequence
  A synthetic peptide
  SITE 8: Xaa = pS (phosphorylated serine)
VKLRSPKX

SEQ ID NO: 464 - protein, 13 aa, Artificial Sequence
  A synthetic peptide
  SITE 8: Xaa = pS (phosphorylated serine)
EYLERRAXRRRAV

SEQ ID NO: 465 - protein, 12 aa, Artificial Sequence
  A synthetic peptide
  SITE 8: Xaa = pS (phosphorylated serine)
ARPKTRIXNKYR

SEQ ID NO: 466 - protein, 12 aa, Artificial Sequence
  A synthetic peptide
  SITE 8: Xaa = pS (phosphorylated serine)
SPYNRKGXFRKQ

SEQ ID NO: 467 - protein, 12 aa, Artificial Sequence
  A synthetic peptide
  SITE 8: Xaa = pS (phosphorylated serine)
ITDESRGXIRRK

SEQ ID NO: 468 - protein, 14 aa, Artificial Sequence
  A synthetic peptide
  SITE 8: Xaa = pS (phosphorylated serine)
PGDGEKRXRIKKSK

SEQ ID NO: 469 - protein, 12 aa, Artificial Sequence
  A synthetic peptide
  SITE 8: Xaa = pS (phosphorylated serine)
KRSRIKKXKKRK

SEQ ID NO: 470 - protein, 12 aa, Artificial Sequence
  A synthetic peptide
  SITE 8: Xaa = pS (phosphorylated serine)
ARLMRRNXLNRK

SEQ ID NO: 471 - protein, 13 aa, Artificial Sequence
  A synthetic peptide
  SITE 7: Xaa = pT (phosphorylated threonine)
RQGKRKXSIKRDT

SEQ ID NO: 472 - protein, 14 aa, Artificial Sequence
  A synthetic peptide
  SITE 8: Xaa = pS (phosphorylated serine)
RQGKRKTXIKRDTV

SEQ ID NO: 473 - protein, 13 aa, Artificial Sequence
  A synthetic peptide
  SITE 8: Xaa = pS (phosphorylated serine)
NRKKKRTXFKRKA

SEQ ID NO: 474 - protein, 14 aa, Artificial Sequence
  A synthetic peptide
KKKKKRFSFKKSFK

SEQ ID NO: 475 - protein, 11 aa, Artificial Sequence
  A synthetic peptide
  SITE (1)...(11): Xaa = any amino acid
XXXXRXXSFDX

SEQ ID NO: 476 - protein, 11 aa, Artificial Sequence
  A synthetic peptide
  SITE (1)...(11): Xaa = any amino acid
XXXXRXXSFNX

SEQ ID NO: 477 - protein, 11 aa, Artificial Sequence
  A synthetic peptide
  SITE (1)...(11): Xaa = any amino acid
XXXXRXXSFQX

SEQ ID NO: 478 - protein, 11 aa, Artificial Sequence
  A synthetic peptide
  SITE (1)...(11): Xaa = any amino acid
XXXXRXXSFRX

SEQ ID NO: 479 - protein, 11 aa, Artificial Sequence
  A synthetic peptide
  SITE (1)...(11): Xaa = any amino acid
XXXXRXXSFKX

SEQ ID NO: 480 - protein, 11 aa, Artificial Sequence
  A synthetic peptide
  SITE (1)...(11): Xaa = any amino acid
XXXXRXXSFHX

SEQ ID NO: 481 - protein, 11 aa, Artificial Sequence
  A synthetic peptide
  SITE (1)...(11): Xaa = any amino acid
XXXXRXXSFSX

SEQ ID NO: 482 - protein, 11 aa, Artificial Sequence
  A synthetic peptide
  SITE (1)...(11): Xaa = any amino acid
XXXXRXXSFPX

SEQ ID NO: 483 - protein, 11 aa, Artificial Sequence
  A synthetic peptide
  SITE (1)...(11): Xaa = any amino acid
XXXXRXXSFLX

SEQ ID NO: 484 - protein, 11 aa, Artificial Sequence
  A synthetic peptide
  SITE (1)...(11): Xaa = any amino acid
XXXXRXXSFFX

SEQ ID NO: 485 - protein, 11 aa, Artificial Sequence
  A synthetic peptide
  SITE (1)...(11): Xaa = any amino acid
XXXXRXXSFWX

SEQ ID NO: 486 - protein, 11 aa, Artificial Sequence
  A synthetic peptide
  SITE (1)...(11): Xaa = any amino acid
XXXXRXXSFGX

SEQ ID NO: 487 - protein, 11 aa, Artificial Sequence
  A synthetic peptide
  SITE (1)...(11): Xaa = any amino acid
XXXXRXXSFAX

SEQ ID NO: 488 - protein, 11 aa, Artificial Sequence
  A synthetic peptide
  SITE (1)...(11): Xaa = any amino acid
XXXDRXXSFXX

SEQ ID NO: 489 - protein, 11 aa, Artificial Sequence
  A synthetic peptide
  SITE (1)...(11): Xaa = any amino acid
XXXNRXXSFXX

SEQ ID NO: 490 - protein, 11 aa, Artificial Sequence
  A synthetic peptide
  SITE (1)...(11): Xaa = any amino acid
XXXQRXXSFXX

SEQ ID NO: 491 - protein, 11 aa, Artificial Sequence
  A synthetic peptide
  SITE (1)...(11): Xaa = any amino acid
XXXRRXXSFXX

SEQ ID NO: 492 - protein, 11 aa, Artificial Sequence
  A synthetic peptide
  SITE (1)...(11): Xaa = any amino acid
XXXKRXXSFXX

SEQ ID NO: 493 - protein, 11 aa, Artificial Sequence
  A synthetic peptide
  SITE (1)...(11): Xaa = any amino acid
XXXHRXXSFXX

SEQ ID NO: 494 - protein, 11 aa, Artificial Sequence
  A synthetic peptide
  SITE (1)...(11): Xaa = any amino acid
XXXSRXXSFXX

SEQ ID NO: 495 - protein, 11 aa, Artificial Sequence
  A synthetic peptide
  SITE (1)...(11): Xaa = any amino acid
XXXPRXXSFXX

SEQ ID NO: 496 - protein, 11 aa, Artificial Sequence
  A synthetic peptide
  SITE (1)...(11): Xaa = any amino acid
XXXLRXXSFXX

SEQ ID NO: 497 - protein, 11 aa, Artificial Sequence
  A synthetic peptide
  SITE (1)...(11): Xaa = any amino acid
XXXFRXXSFXX

SEQ ID NO: 498 - protein, 11 aa, Artificial Sequence
  A synthetic peptide
  SITE (1)...(11): Xaa = any amino acid
XXXVRXXSFXX

SEQ ID NO: 499 - protein, 11 aa, Artificial Sequence
  A synthetic peptide
  SITE (1)...(11): Xaa = any amino acid
XXXGRXXSFXX

SEQ ID NO: 500 - protein, 11 aa, Artificial Sequence
  A synthetic peptide
  SITE (1)...(11): Xaa = any amino acid
XXXARXXSFXX

SEQ ID NO: 501 - protein, 11 aa, Artificial Sequence
  A synthetic peptide
  SITE (1)...(11): Xaa = any amino acid
XXXXRDXSFXX

SEQ ID NO: 502 - protein, 11 aa, Artificial Sequence
  A synthetic peptide
  SITE (1)...(11): Xaa = any amino acid
XXXXRNXSFXX

SEQ ID NO: 503 - protein, 11 aa, Artificial Sequence
  A synthetic peptide
  SITE (1)...(11): Xaa = any amino acid
XXXXRQXSFXX

SEQ ID NO: 504 - protein, 11 aa, Artificial Sequence
  A synthetic peptide
  SITE (1)...(11): Xaa = any amino acid
XXXXRRXSFXX

SEQ ID NO: 505 - protein, 11 aa, Artificial Sequence
  A synthetic peptide
  SITE (1)...(11): Xaa = any amino acid
XXXXRKXSFXX

SEQ ID NO: 506 - protein, 11 aa, Artificial Sequence
  A synthetic peptide
  SITE (1)...(11): Xaa = any amino acid
XXXXRHXSFXX

SEQ ID NO: 507 - protein, 11 aa, Artificial Sequence
  A synthetic peptide
  SITE (1)...(11): Xaa = any amino acid
XXXXRSXSFXX

SEQ ID NO: 508 - protein, 11 aa, Artificial Sequence
  A synthetic peptide
  SITE (1)...(11): Xaa = any amino acid
XXXXRPXSFXX

SEQ ID NO: 509 - protein, 11 aa, Artificial Sequence
  A synthetic peptide
  SITE (1)...(11): Xaa = any amino acid
XXXXRLXSFXX

SEQ ID NO: 510 - protein, 11 aa, Artificial Sequence
  A synthetic peptide
  SITE (1)...(11): Xaa = any amino acid
XXXXRFXSFXX

SEQ ID NO: 511 - protein, 11 aa, Artificial Sequence
  A synthetic peptide
  SITE (1)...(11): Xaa = any amino acid
XXXXRVXSFXX

SEQ ID NO: 512 - protein, 11 aa, Artificial Sequence
  A synthetic peptide
  SITE (1)...(11): Xaa = any amino acid
XXXXRGXSFXX

SEQ ID NO: 513 - protein, 11 aa, Artificial Sequence
  A synthetic peptide
  SITE (1)...(11): Xaa = any amino acid
XXXXRAXSFXX

SEQ ID NO: 514 - protein, 11 aa, Artificial Sequence
  A synthetic peptide
  SITE (1)...(11): Xaa = any amino acid
XXXXRXDSFXX

SEQ ID NO: 515 - protein, 11 aa, Artificial Sequence
  A synthetic peptide
  SITE (1)...(11): Xaa = any amino acid
XXXXRXNSFXX

SEQ ID NO: 516 - protein, 11 aa, Artificial Sequence
  A synthetic peptide
  SITE (1)...(11): Xaa = any amino acid
XXXXRXQSFXX

SEQ ID NO: 517 - protein, 11 aa, Artificial Sequence
  A synthetic peptide
  SITE (1)...(11): Xaa = any amino acid
XXXXRXRSFXX

SEQ ID NO: 518 - protein, 11 aa, Artificial Sequence
  A synthetic peptide
  SITE (1)...(11): Xaa = any amino acid
XXXXRXKSFXX

SEQ ID NO: 519 - protein, 11 aa, Artificial Sequence
  A synthetic peptide
  SITE (1)...(11): Xaa = any amino acid
XXXXRXHSFXX

SEQ ID NO: 520 - protein, 11 aa, Artificial Sequence
  A synthetic peptide
  SITE (1)...(11): Xaa = any amino acid
XXXXRXSSFXX

SEQ ID NO: 521 - protein, 11 aa, Artificial Sequence
  A synthetic peptide
  SITE (1)...(11): Xaa = any amino acid
XXXXRXPSFXX

SEQ ID NO: 522 - protein, 11 aa, Artificial Sequence
  A synthetic peptide
  SITE (1)...(11): Xaa = any amino acid
XXXXRXLSFXX

SEQ ID NO: 523 - protein, 11 aa, Artificial Sequence
  A synthetic peptide
  SITE (1)...(11): Xaa = any amino acid
XXXXRXFSFXX

SEQ ID NO: 524 - protein, 11 aa, Artificial Sequence
  A synthetic peptide
  SITE (1)...(11): Xaa = any amino acid
XXXXRXVSFXX

SEQ ID NO: 525 - protein, 11 aa, Artificial Sequence
  A synthetic peptide
  SITE (1)...(11): Xaa = any amino acid
XXXXRXGSFXX

SEQ ID NO: 526 - protein, 11 aa, Artificial Sequence
  A synthetic peptide
  SITE (1)...(11): Xaa = any amino acid
XXXXRXASFXX

SEQ ID NO: 527 - protein, 11 aa, Artificial Sequence
  A synthetic peptide
  SITE (1)...(11): Xaa = any amino acid
XXXXRXXSFDX

SEQ ID NO: 528 - protein, 11 aa, Artificial Sequence
  A synthetic peptide
  SITE (1)...(11): Xaa = any amino acid
XXXXRXXSFNX

SEQ ID NO: 529 - protein, 11 aa, Artificial Sequence
  A synthetic peptide
  SITE (1)...(11): Xaa = any amino acid
XXXXRXXSFQX

SEQ ID NO: 530 - protein, 11 aa, Artificial Sequence
  A synthetic peptide
  SITE (1)...(11): Xaa = any amino acid
XXXXRXXSFRX

SEQ ID NO: 531 - protein, 11 aa, Artificial Sequence
  A synthetic peptide
  SITE (1)...(11): Xaa = any amino acid
XXXXRXXSFKX

SEQ ID NO: 532 - protein, 11 aa, Artificial Sequence
  A synthetic peptide
  SITE (1)...(11): Xaa = any amino acid
XXXXRXXSFHX

SEQ ID NO: 533 - protein, 11 aa, Artificial Sequence
  A synthetic peptide
  SITE (1)...(11): Xaa = any amino acid
XXXXRXXSFSX

SEQ ID NO: 534 - protein, 11 aa, Artificial Sequence
  A synthetic peptide
  SITE (1)...(11): Xaa = any amino acid
XXXXRXXSFPX

SEQ ID NO: 535 - protein, 11 aa, Artificial Sequence
  A synthetic peptide
  SITE (1)...(11): Xaa = any amino acid
XXXXRXXSFLX

SEQ ID NO: 536 - protein, 11 aa, Artificial Sequence
  A synthetic peptide
  SITE (1)...(11): Xaa = any amino acid
XXXXRXXSFFX

SEQ ID NO: 537 - protein, 11 aa, Artificial Sequence
  A synthetic peptide
  SITE (1)...(11): Xaa = any amino acid
XXXXRXXSFVX

SEQ ID NO: 538 - protein, 11 aa, Artificial Sequence
  A synthetic peptide
  SITE (1)...(11): Xaa = any amino acid
XXXXRXXSFGX

SEQ ID NO: 539 - protein, 11 aa, Artificial Sequence
  A synthetic peptide
  SITE (1)...(11): Xaa = any amino acid
XXXXRXXSFAX

SEQ ID NO: 540 - protein, 11 aa, Artificial Sequence
  A synthetic peptide
  SITE (1)...(11): Xaa = any amino acid
XXXXRXXSFXD

SEQ ID NO: 541 - protein, 11 aa, Artificial Sequence
  A synthetic peptide
  SITE (1)...(11): Xaa = any amino acid
XXXXRXXSFXN

SEQ ID NO: 542 - protein, 11 aa, Artificial Sequence
  A synthetic peptide
  SITE (1)...(11): Xaa = any amino acid
XXXXRXXSFXQ

SEQ ID NO: 543 - protein, 11 aa, Artificial Sequence
  A synthetic peptide
  SITE (1)...(11): Xaa = any amino acid
XXXXRXXSFXR

SEQ ID NO: 544 - protein, 11 aa, Artificial Sequence
  A synthetic peptide
  SITE (1)...(11): Xaa = any amino acid
XXXXRXXSFXK

SEQ ID NO: 545 - protein, 11 aa, Artificial Sequence
  A synthetic peptide
  SITE (1)...(11): Xaa = any amino acid
XXXXRXXSFXH

SEQ ID NO: 546 - protein, 11 aa, Artificial Sequence
  A synthetic peptide
  SITE (1)...(11): Xaa = any amino acid
XXXXRXXSFXS

SEQ ID NO: 547 - protein, 11 aa, Artificial Sequence
  A synthetic peptide
  SITE (1)...(11): Xaa = any amino acid
XXXXRXXSFXP

SEQ ID NO: 548 - protein, 11 aa, Artificial Sequence
  A synthetic peptide
  SITE (1)...(11): Xaa = any amino acid
XXXXRXXSFXL

SEQ ID NO: 549 - protein, 11 aa, Artificial Sequence
  A synthetic peptide
  SITE (1)...(11): Xaa = any amino acid
XXXXRXXSFXF

SEQ ID NO: 550 - protein, 11 aa, Artificial Sequence
  A synthetic peptide
  SITE (1)...(11): Xaa = any amino acid
XXXXRXXSFXV

SEQ ID NO: 551 - protein, 11 aa, Artificial Sequence
  A synthetic peptide
  SITE (1)...(11): Xaa = any amino acid
XXXXRXXSFXG

SEQ ID NO: 552 - protein, 11 aa, Artificial Sequence
  A synthetic peptide
  SITE (1)...(11): Xaa = any amino acid
XXXXRXXSFXA

SEQ ID NO: 553 - skipped

SEQ ID NO: 554 - protein, 12 aa, Artificial Sequence
  A synthetic peptide
  SITE (1)...(12): Xaa = any amino acid
XDXXXRXXSFXX

SEQ ID NO: 555 - protein, 12 aa, Artificial Sequence
  A synthetic peptide
  SITE (1)...(12): Xaa = any amino acid
XNXXXRXXSFXX

SEQ ID NO: 556 - protein, 12 aa, Artificial Sequence
  A synthetic peptide
  SITE (1)...(12): Xaa = any amino acid
XQXXXRXXSFXX

SEQ ID NO: 557 - protein, 12 aa, Artificial Sequence
  A synthetic peptide
  SITE (1)...(12): Xaa = any amino acid
XRXXXRXXSFXX

SEQ ID NO: 558 - protein, 12 aa, Artificial Sequence
  A synthetic peptide
  SITE (1)...(12): Xaa = any amino acid
XKXXXRXXSFXX

SEQ ID NO: 559 - protein, 12 aa, Artificial Sequence
  A synthetic peptide
  SITE (1)...(12): Xaa = any amino acid
XHXXXRXXSFXX

SEQ ID NO: 560 - protein, 12 aa, Artificial Sequence
  A synthetic peptide
  SITE (1)...(12): Xaa = any amino acid
XSXXXRXXSFXX

SEQ ID NO: 561 - protein, 12 aa, Artificial Sequence
  A synthetic peptide
  SITE (1)...(12): Xaa = any amino acid
XPXXXRXXSFXX

SEQ ID NO: 562 - protein, 12 aa, Artificial Sequence
  A synthetic peptide
  SITE (1)...(12): Xaa = any amino acid
XLXXXRXXSFXX

SEQ ID NO: 563 - protein, 12 aa, Artificial Sequence
  A synthetic peptide
  SITE (1)...(12): Xaa = any amino acid
XFXXXRXXSFXX

SEQ ID NO: 564 - protein, 12 aa, Artificial Sequence
  A synthetic peptide
  SITE (1)...(12): Xaa = any amino acid
XVXXXRXXSFXX

SEQ ID NO: 565 - protein, 12 aa, Artificial Sequence
  A synthetic peptide
  SITE (1)...(12): Xaa = any amino acid
XGXXXRXXSFXX

SEQ ID NO: 566 - protein, 12 aa, Artificial Sequence
  A synthetic peptide
  SITE (1)...(12): Xaa = any amino acid
XAXXXRXXSFXX

SEQ ID NO: 567 - protein, 12 aa, Artificial Sequence
  A synthetic peptide
  SITE (1)...(12): Xaa = any amino acid
XXDXXRXXSFXX

SEQ ID NO: 568 - protein, 12 aa, Artificial Sequence
  A synthetic peptide
  SITE (1)...(12): Xaa = any amino acid
XXNXXRXXSFXX

SEQ ID NO: 569 - protein, 12 aa, Artificial Sequence
  A synthetic peptide
  SITE (1)...(12): Xaa = any amino acid
XXQXXRXXSFXX

SEQ ID NO: 570 - protein, 12 aa, Artificial Sequence
  A synthetic peptide
  SITE (1)...(12): Xaa = any amino acid
XXRXXRXXSFXX

SEQ ID NO: 571 - protein, 12 aa, Artificial Sequence
  A synthetic peptide
  SITE (1)...(12): Xaa = any amino acid
XXKXXRXXSFXX

SEQ ID NO: 572 - protein, 12 aa, Artificial Sequence
  A synthetic peptide
  SITE (1)...(12): Xaa = any amino acid
XXHXXRXXSFXX

SEQ ID NO: 573 - protein, 12 aa, Artificial Sequence
  A synthetic peptide
  SITE (1)...(12): Xaa = any amino acid
XXSXXRXXSFXX

SEQ ID NO: 574 - protein, 12 aa, Artificial Sequence
  A synthetic peptide
  SITE (1)...(12): Xaa = any amino acid
XXPXXRXXSFXX

SEQ ID NO: 575 - protein, 12 aa, Artificial Sequence
  A synthetic peptide
  SITE (1)...(12): Xaa = any amino acid
XXLXXRXXSFXX

SEQ ID NO: 576 - protein, 12 aa, Artificial Sequence
  A synthetic peptide
  SITE (1)...(12): Xaa = any amino acid
XXFXXRXXSFXX

SEQ ID NO: 577 - protein, 12 aa, Artificial Sequence
  A synthetic peptide
  SITE (1)...(12): Xaa = any amino acid
XXVXXRXXSFXX

SEQ ID NO: 578 - protein, 12 aa, Artificial Sequence
  A synthetic peptide
  SITE (1)...(12): Xaa = any amino acid
XXGXXRXXSFXX

SEQ ID NO: 579 - protein, 12 aa, Artificial Sequence
  A synthetic peptide
  SITE (1)...(12): Xaa = any amino acid
XXAXXRXXSFXX

SEQ ID NO: 580 - protein, 12 aa, Artificial Sequence
  A synthetic peptide
  SITE (1)...(12): Xaa = any amino acid
XXXDXRXXSFXX

SEQ ID NO: 581 - protein, 12 aa, Artificial Sequence
  A synthetic peptide
  SITE (1)...(12): Xaa = any amino acid
XXXNXRXXSFXX

SEQ ID NO: 582 - protein, 12 aa, Artificial Sequence
  A synthetic peptide
  SITE (1)...(12): Xaa = any amino acid
XXXQXRXXSFXX

SEQ ID NO: 583 - protein, 12 aa, Artificial Sequence
  A synthetic peptide
  SITE (1)...(12): Xaa = any amino acid
XXXRXRXXSFXX

SEQ ID NO: 584 - protein, 12 aa, Artificial Sequence
  A synthetic peptide
  SITE (1)...(12): Xaa = any amino acid
XXXKXRXXSFXX

SEQ ID NO: 585 - protein, 12 aa, Artificial Sequence
  A synthetic peptide
  SITE (1)...(12): Xaa = any amino acid
XXXHXRXXSFXX

SEQ ID NO: 586 - protein, 12 aa, Artificial Sequence
  A synthetic peptide
  SITE (1)...(12): Xaa = any amino acid
XXXSXRXXSFXX

SEQ ID NO: 587 - protein, 12 aa, Artificial Sequence
  A synthetic peptide
  SITE (1)...(12): Xaa = any amino acid
XXXPXRXXSFXX

SEQ ID NO: 588 - protein, 12 aa, Artificial Sequence
  A synthetic peptide
  SITE (1)...(12): Xaa = any amino acid
XXXLXRXXSFXX

SEQ ID NO: 589 - protein, 12 aa, Artificial Sequence
  A synthetic peptide
  SITE (1)...(12): Xaa = any amino acid
XXXFXRXXSFXX

SEQ ID NO: 590 - protein, 12 aa, Artificial Sequence
  A synthetic peptide
  SITE (1)...(12): Xaa = any amino acid
XXXVXRXXSFXX

SEQ ID NO: 591 - protein, 12 aa, Artificial Sequence
  A synthetic peptide
  SITE (1)...(12): Xaa = any amino acid
XXXGXRXXSFXX

SEQ ID NO: 592 - protein, 12 aa, Artificial Sequence
  A synthetic peptide
  SITE (1)...(12): Xaa = any amino acid
XXXAXRXXSFXX

SEQ ID NO: 593 - protein, 12 aa, Artificial Sequence
  A synthetic peptide
  SITE (1)...(12): Xaa = any amino acid
XXRXXSFXXDXX

SEQ ID NO: 594 - protein, 12 aa, Artificial Sequence
  A synthetic peptide
  SITE (1)...(12): Xaa = any amino acid
XXRXXSFXXNXX

SEQ ID NO: 595 - protein, 12 aa, Artificial Sequence
  A synthetic peptide
  SITE (1)...(12): Xaa = any amino acid
XXRXXSFXXQXX

SEQ ID NO: 596 - protein, 12 aa, Artificial Sequence
  A synthetic peptide
  SITE (1)...(12): Xaa = any amino acid
XXRXXSFXXRXX

SEQ ID NO: 597 - protein, 12 aa, Artificial Sequence
  A synthetic peptide
  SITE (1)...(12): Xaa = any amino acid
XXRXXSFXXKXX

SEQ ID NO: 598 - protein, 12 aa, Artificial Sequence
  A synthetic peptide
  SITE (1)...(12): Xaa = any amino acid
XXRXXSFXXHXX

SEQ ID NO: 599 - protein, 12 aa, Artificial Sequence
  A synthetic peptide
  SITE (1)...(12): Xaa = any amino acid
XXRXXSFXXSXX

SEQ ID NO: 600 - protein, 12 aa, Artificial Sequence
  A synthetic peptide
  SITE (1)...(12): Xaa = any amino acid
XXRXXSFXXPXX

SEQ ID NO: 601 - protein, 12 aa, Artificial Sequence
  A synthetic peptide
  SITE (1)...(12): Xaa = any amino acid
XXRXXSFXXLXX

SEQ ID NO: 602 - protein, 12 aa, Artificial Sequence
  A synthetic peptide
  SITE (1)...(12): Xaa = any amino acid
XXRXXSFXXFXX

SEQ ID NO: 603 - protein, 12 aa, Artificial Sequence
  A synthetic peptide
  SITE (1)...(12): Xaa = any amino acid
XXRXXSFXXVXX

SEQ ID NO: 604 - protein, 12 aa, Artificial Sequence
  A synthetic peptide
  SITE (1)...(12): Xaa = any amino acid
XXRXXSFXXGXX

SEQ ID NO: 605 - protein, 12 aa, Artificial Sequence
  A synthetic peptide
  SITE (1)...(12): Xaa = any amino acid
XXRXXSFXXAXX

SEQ ID NO: 606 - protein, 12 aa, Artificial Sequence
  A synthetic peptide
  SITE (1)...(12): Xaa = any amino acid
XXRXXSFXXXDX

SEQ ID NO: 607 - protein, 12 aa, Artificial Sequence
  A synthetic peptide
  SITE (1)...(12): Xaa = any amino acid
XXRXXSFXXXNX

SEQ ID NO: 608 - protein, 12 aa, Artificial Sequence
  A synthetic peptide
  SITE (1)...(12): Xaa = any amino acid
XXRXXSFXXXQX

SEQ ID NO: 609 - protein, 12 aa, Artificial Sequence
  A synthetic peptide
  SITE (1)...(12): Xaa = any amino acid
XXRXXSFXXXRX

SEQ ID NO: 610 - protein, 12 aa, Artificial Sequence
  A synthetic peptide
  SITE (1)...(12): Xaa = any amino acid
XXRXXSFXXXKX

SEQ ID NO: 611 - protein, 12 aa, Artificial Sequence
  A synthetic peptide
  SITE (1)...(12): Xaa = any amino acid
XXRXXSFXXXHX

SEQ ID NO: 612 - protein, 12 aa, Artificial Sequence
  A synthetic peptide
  SITE (1)...(12): Xaa = any amino acid
XXRXXSFXXXSX

SEQ ID NO: 613 - protein, 12 aa, Artificial Sequence
  A synthetic peptide
  SITE (1)...(12): Xaa = any amino acid
XXRXXSFXXXPX

SEQ ID NO: 614 - protein, 12 aa, Artificial Sequence
  A synthetic peptide
  SITE (1)...(12): Xaa = any amino acid
XXRXXSFXXXLX

SEQ ID NO: 615 - protein, 12 aa, Artificial Sequence
  A synthetic peptide
  SITE (1)...(12): Xaa = any amino acid
XXRXXSFXXXFX

SEQ ID NO: 616 - protein, 12 aa, Artificial Sequence
  A synthetic peptide
  SITE (1)...(12): Xaa = any amino acid
XXRXXSFXXXVX

SEQ ID NO: 617 - protein, 12 aa, Artificial Sequence
  A synthetic peptide
  SITE (1)...(12): Xaa = any amino acid
XXRXXSFXXXGX

SEQ ID NO: 618 - protein, 12 aa, Artificial Sequence
  A synthetic peptide
  SITE (1)...(12): Xaa = any amino acid
XXRXXSFXXXAX

SEQ ID NO: 619 - protein, 12 aa, Artificial Sequence
  A synthetic peptide
  SITE (1)...(12): Xaa = any amino acid
XXRXXSFXXXXD

SEQ ID NO: 620 - protein, 12 aa, Artificial Sequence
  A synthetic peptide
  SITE (1)...(12): Xaa = any amino acid
XXRXXSFXXXXN

SEQ ID NO: 621 - protein, 12 aa, Artificial Sequence
  A synthetic peptide
  SITE (1)...(12): Xaa = any amino acid
XXRXXSFXXXXQ

SEQ ID NO: 622 - protein, 12 aa, Artificial Sequence
  A synthetic peptide
  SITE (1)...(12): Xaa = any amino acid
XXRXXSFXXXXR

SEQ ID NO: 623 - protein, 12 aa, Artificial Sequence
  A synthetic peptide
  SITE (1)...(12): Xaa = any amino acid
XXRXXSFXXXXK

SEQ ID NO: 624 - protein, 12 aa, Artificial Sequence
  A synthetic peptide
  SITE (1)...(12): Xaa = any amino acid
XXRXXSFXXXXH

SEQ ID NO: 625 - protein, 12 aa, Artificial Sequence
  A synthetic peptide
  SITE (1)...(12): Xaa = any amino acid
XXRXXSFXXXXS

SEQ ID NO: 626 - protein, 12 aa, Artificial Sequence
  A synthetic peptide
  SITE (1)...(12): Xaa = any amino acid
XXRXXSFXXXXP

SEQ ID NO: 627 - protein, 12 aa, Artificial Sequence
  A synthetic peptide
  SITE (1)...(12): Xaa = any amino acid
XXRXXSFXXXXL

SEQ ID NO: 628 - protein, 12 aa, Artificial Sequence
  A synthetic peptide
  SITE (1)...(12): Xaa = any amino acid
XXRXXSFXXXXF

SEQ ID NO: 629 - protein, 12 aa, Artificial Sequence
  A synthetic peptide
  SITE (1)...(12): Xaa = any amino acid
XXRXXSFXXXXV

SEQ ID NO: 630 - protein, 12 aa, Artificial Sequence
  A synthetic peptide
  SITE (1)...(12): Xaa = any amino acid
XXRXXSFXXXXG

SEQ ID NO: 631 - protein, 12 aa, Artificial Sequence
  A synthetic peptide
  SITE (1)...(12): Xaa = any amino acid
XXRXXSFXXXXA

SEQ ID NO: 632 - protein, 17 aa, Artificial Sequence
  A synthetic peptide
KAEDGATPSPSNETPKK

SEQ ID NO: 633 - protein, 17 aa, Artificial Sequence
  A synthetic peptide
EDGATPSPSNETPKKKK

SEQ ID NO: 634 - protein, 17 aa, Artificial Sequence
  A synthetic peptide
ATPSPSNETPKKKKKRF

SEQ ID NO: 635 - protein, 17 aa, Artificial Sequence
  A synthetic peptide
PKKKKKRFSFKKSFKLS

SEQ ID NO: 636 - protein, 17 aa, Artificial Sequence
  A synthetic peptide
KKRFSFKKSFKLSGFSF

SEQ ID NO: 637 - protein, 17 aa, Artificial Sequence
  A synthetic peptide
SFKKSFKLSGFSFKKNK

SEQ ID NO: 638 - protein, 17 aa, Artificial Sequence
  A synthetic peptide
KSFKLSGFSFKKNKKEA

SEQ ID NO: 639 - protein, 17 aa, Artificial Sequence
  A synthetic peptide
AAAEAGAASGEQAAAPG

SEQ ID NO: 640 - protein, 17 aa, Artificial Sequence
  A synthetic peptide
VAPEKPPASDETKAAEE

(19) United States
(12) Patent Application Publication
(10) Pub. No.: US 2005/0064507 A1
(43) Pub. Date: Mar. 24, 2005

(54) Determining kinase specificity

(76) Inventor: James Stephen Shaw, Bethesda, MD (US)

Correspondence Address: Schwegman, Lundberg, Woessner & Kluth, P.A., P.O. Box 2938, Minneapolis, MN, 55402, US

(21) Appl. No.: 10/660,370

(22) Filed: Sep. 11, 2003

(51) Int. Cl. G01N 33/53; C12Q 1/48

(52) U.S. Cl. 435/7.1; 435/15

(57) ABSTRACT

The invention provides methods, articles, software, kits as well as sets and arrays of peptides for determining the spectrum of peptidyl sequences that are recognized and phosphorylated by a kinase.

### FIELD OF THE INVENTION

The invention relates to methods, articles, software and kits for determining the spectrum of peptidyl sequences that are recognized and phosphorylated by a kinase.

### BACKGROUND OF THE INVENTION

The activity of cells is regulated by external signals that stimulate or inhibit intracellular events. The process by which stimulatory or inhibitory signals are transmitted into and within a cell to elicit an intracellular response is referred to as signal transduction. Proper signal transduction is essential for proper cellular function. Defects in various components of signal transduction pathways, from cell surface receptors to activators of gene transcription, account for a vast number of diseases, including numerous forms of cancer, vascular diseases and neuronal diseases.

Signal transduction is largely mediated by protein kinases. Protein kinases are enzymes that phosphorylate other proteins and/or themselves (auto-phosphorylation). A major rate-limiting problem in understanding signal transduction within cells is to determine which kinase phosphorylates which protein substrate at which sites within the protein substrate.

Eukaryotic protein kinases are numerous and diverse; there are more than 500 human genes than encode different protein kinases (Manning G et al. 2002. Science 298:1912-1934). Eukaryotic protein kinases that are involved in signal transduction can be divided into three major groups based upon their substrate utilization. First, the protein-tyrosine specific kinases can phosphorylate substrates on tyrosine residues. Second, the protein-serine/threonine specific kinases can phosphorylate substrates at serine and/or threonine residues. Finally, the dual-specificity kinases can phosphorylate substrates at tyrosine, serine and/or threonine residues.

In order to insure fidelity in intracellular signal transduction cascades it is essential that each protein kinase have exquisite specificity for its target substrate(s). In general, kinases appear to phosphorylate multiple different target sites on multiple proteins, thereby allowing branching of an initial signal delivered to a cell in multiple directions in order to coordinate a set of events that occur in parallel for a given cellular response (see, for example, Roach, P. J. (1991) J. Biol. Chem. 266:14139-14142).

The substrate specificity of a protein kinase can be influenced by at least three general mechanisms that depend on the overall structure of the enzyme. First, specific domains in certain protein kinases can target the kinase to specific locations in the cell, thereby restricting the substrate availability of the kinase. Second, domains in the kinase, distinct from its catalytic domain, may provide high affinity association with either the substrate or an adapter molecule that presents the substrate to the kinase. Finally, kinase specificity is ultimately provided by the structure of the catalytic site of the protein kinase that drives it to select one peptide substrate sequence over another.

Although the number of protein kinases that have been implicated in intracellular signaling is quite large, detailed information about the sequence specificity of these kinases is available for only a limited number of these kinases. Shortcomings in the available approaches for detailed characterization of kinase specificity are largely responsible for this scarcity of information. One systematic approach to characterization of kinase specificity involves collecting information on many specific substrates for a kinase and determining common features amongst the substrates sequences (Kreegipuu A et al. 1998. FEBS Lett 430:45-50). Such determination of the individual substrates is a laborious and largely empirical process, making this a slow and relatively inefficient way to derive comprehensive information on kinase specificity.

In the early 1990s, Cantley and colleagues invented a method that attempts to accurately predict the spectrum of good peptide substrates for a kinase (see U.S. Pat. No. 5,532,167; Songyang Z et al. 1994. Curr Biol 4:973-982). Predictions of substrate specificity made by this method are available at a website at scansite.mit.edu/. See also Obenauer J C et al. 2003. Nucleic Acids Res 31:3635-3641; Yaffe M B et al. 2001. Nat Biotechnol 19:348-353. Other workers have employed what can be referred to as “systematic amino acid variation on template substrate” (SAaVoTS) to describe a class of approaches that analyze kinase specificity by synthesizing sets of peptides using a strategy of systematic variation of residues on a “template sequence.” The simplest template for SAaVoTS is a known substrate. See, Himpel S et al. 2000. J Biol Chem 275:2431-2438, Velentza A V et al. 2001. J Biol Chem 276:38956-38965. A second variation on this “systematic amino acid variation on template substrate” (SAaVoTS) involves looking for an optimal peptide substrate sequence (Dostmann W R et al. 1999. Pharmacol Ther 82:373-387; Tegge W J et al. 1998. Methods Mol Biol 87:99-106; Tegge W et al. 1995. Biochemistry 34:10569-10577).

Limitations typical of these previous approaches therefore include a failure to thoroughly validate their findings, a propensity for seeking optimal substrate sequences rather than defining the universe of preferred substrates, and/or assumptions that a method provides general information when it may provide rather narrow information. Thus, there is a need for an alternative method to characterize the universe of preferred substrates for kinases.

### SUMMARY OF THE INVENTION

The invention relates to determination of the range of substrate specificities of protein kinases, to visual representation of those kinase specificities, to prediction of sites on sequenced proteins that are most likely to be phosphorylated by each kinase studied, to validation in vitro that peptides corresponding to those predicted sites are indeed phosphorylated by each kinase studied, and to validation of phosphorylation of those sites in vivo. The invention provides a simple and efficient method for determining the amino acid residue preferences for peptidyl sequences phosphorylated by a kinase, as well as for predicting which sites will be preferentially phosphorylated by the kinase, and software that facilitates those methods. The invention also provides an informative graphical format for visually representing that information and software to output data in that format. Peptide sequences proven to be well phosphorylated by protein kinase C are also provided.

In one embodiment, the invention provides a test set of peptide pools for identifying kinase substrate specificities. Such a test set for characterizing substrate specificities of kinases has at least two peptide pools. In general, substantially every peptide in each of the peptide pools includes one defined phosphorylatable amino acid position, one query amino acid position, at least one anchor amino acid position, and at least one degenerate amino acid position. Substantially every peptide of every peptide pool has an identical phosphorylatable amino acid that can be phosphorylated by a kinase at the phosphorylatable amino acid position. The query amino acid position is at a defined position relative to the phosphorylatable amino acid position within substantially every peptide of every peptide pool, but a query amino acid's identity at the query amino acid position is systematically varied from one peptide pool to the next peptide pool within the test set of peptide pools. Each anchor amino acid position is at a defined position relative to the phosphorylatable amino acid position within substantially every peptide of every peptide pool and each anchor amino acid position has an identical anchor amino acid at that anchor amino acid position within every peptide of every peptide pool. Each degenerate amino acid position within every peptide of every peptide pool is occupied by an amino acid from a defined mixture of amino acids. In some embodiments, the query amino acid position is not adjacent to an anchor amino acid position or the query amino acid position is not adjacent to the phosphorylatable amino acid position in any peptide pool of the test set. In some test sets of the invention, no anchor amino acid positions (or anchor amino acids) are present, however, such test sets do have a phosphorylatable amino acid position, and at least one query amino acid position. Such “anchor-free” test sets will also generally have at least one degenerate amino acid position.

In other embodiments, the invention provides a test set like those described above except that every peptide of every peptide pool has an identical query amino acid but the position of the query amino acid relative to the phosphorylatable amino acid position is systematically varied from one peptide pool to the next peptide pool within the test set of peptide pools. One desirable query amino acid to use in such a test set is arginine.

The invention also provides a binding entity whose binding differentiates between a defined peptide having any one of SEQ ID NO: 76, 79, 81, 82, 87, 89-94, 97, 98, 100, 102, 104, 105, 108, 110, 112, 113, 115, 117, 121, 124, 125, 127-134, 136, 138, 139, 143-145, 148-153, 156, 160, 163-180, 182-194, 196-206, 208-211, 213-216 and the corresponding defined peptide after phosphorylation by PKC-theta, and wherein the binding entity has substantially no binding to a phosphorylated peptide having SEQ ID NO: 229 (WKN-pS-IRH).

The invention further provides a binding entity whose binding differentiates between a defined phosphorylated peptide having any one of SEQ ID NO:298-347, 349-473 and a non-phosphorylated peptide that differs from the defined peptide by substitution of Ser for the pSer or substitution of a Thr for the pThr, and wherein the binding entity has substantially no binding to a phosphorylated peptide having SEQ ID NO: 229 (WKN-pS-IRH).

The invention also provides a method for characterizing substrate specificities of kinases that includes: contacting each peptide pool in at least two test sets of peptide pools with ATP and a kinase; quantifying the amount of phosphorylation in each peptide pool; and comparing the amount of phosphorylation in each peptide pool with the amount of phosphorylation in at least one other peptide pool. Test sets like those described above can be used in the methods of the invention. Comparison of the amount of phosphorylation in different peptide pools of a test set allows calculation of the preferences of the kinase for each query residue, which differs between those pools. By testing multiple test sets (for example, by using a superset described herein), a position specific scoring matrix (PSSM) can be derived, which reflects the amino acid preferences of the kinase at positions around the phosphorylation position.

The methods of the invention are flexible. For example, the same sets of degenerate peptides can be used to characterize many different kinases from every one of the millions of different biological species and an almost unlimited range of mutant kinases derived from each such kinase. Flexibility is also present in the type of phosphorylation sites characterized by the methods of the invention and in the number of query positions and residue types are explored. Moreover, the methods of the invention can also be modulated so that different residues at a single position are tested, or the same residues are tested at different positions. More than 500 peptide pools have been synthesized in more than 40 test sets, belonging to more than 6 supersets.

The invention further provides a computer readable medium that includes computer-executable instructions, wherein the computer-executable instructions comprise conversion of input data into quantitative values specifying a preference value for each of a plurality of amino acids at each defined position in a substrate peptide for a kinase, wherein: the input data comprises sequence and phosphorylation data for a test set of peptides comprising at least two peptide pools, wherein every peptide in each of the peptide pools comprises one phosphorylatable amino acid position, and one query amino acid position, wherein: each peptide of every peptide pool has an identical phosphorylatable amino acid that can be phosphorylated by a kinase at the phosphorylatable amino acid position; the query amino acid position is at the defined position relative to the phosphorylatable amino acid position within every peptide of every peptide pool but a query amino acid's identity at the query amino acid position is systematically varied from one peptide pool to the next peptide pool within the test set of peptide pools; a preference value for a particular amino acid at the defined position is substantially determined from the amount of phosphorylation of the peptide pool wherein that particular amino acid is the query residue and the query position is located at the defined position.

The invention also provides a method for visual display of amino acid or nucleotide sequence preferences comprising a series of stacks of single letter symbols for amino acids or nucleotides, wherein each stack represents a position in a peptide or a nucleic acid sequence; each symbol's height is proportional to the absolute value of a quantitative parameter that is positive for favored amino acids or nucleotides and negative for disfavored amino acids or nucleotides; each symbol's position within the stack is sorted from bottom to top in ascending value by the quantitative parameter.

In another embodiment, the invention provides a computer readable medium having computer-executable instructions for performing a method of visually displaying amino acid or nucleotide sequence preferences, the method comprising: representing a position in a peptide or a nucleic acid sequence with a stack of single letter symbols for amino acids or nucleotides; and displaying a linear array of one or more stacks of letter symbols wherein each letter symbol's height is proportional to the absolute value of a quantitative parameter that is positive for favored amino acids or nucleotides and negative for disfavored amino acids or nucleotides and wherein each letter symbol's position within the stack is sorted from bottom to top in ascending order by the value of the quantitative parameter.

The result of the graphic methods of the invention is a PSSM Logo, which is a novel graphical format for conveying the specificity information in a PSSM. It is particularly efficient in conveying both information on the preferred residues and the disfavored residues, which act in concert to determine the specificity of the kinase.

The present invention provides detailed information on the types of sites and amino acid sequences that are recognized and phosphorylated by a kinase, thereby permitting accurate prediction of which peptide sequences in the human proteome can be phosphorylated by a particular kinase. Hence, computer programs have been used to scan known well-defined human genes (15323). Approximately 1900 human gene products were thereby identified that had at least one Ser/Thr residue that predicted to be phosphorylated by protein kinase C (PKC) using a high stringency prediction criterion (better than 0.2 percentile). The validity of the PSSM derived results with supersets of peptides has been extensively validated by demonstrating an excellent correlation between peptides predicted to be phosphorylated in vitro by a kinase and those that are phosphorylated in vitro by that kinase. Moreover, the biological relevance of the in vitro phosphorylation is supported by comparison of sites identified with a literature search defining sites phosphorylated in vivo.

### BRIEF DESCRIPTION OF THE FIGURES

FIG. 1 provides examples of two test sets of peptide pools and results obtained with PKC-theta using the methods of the invention. (SEQ ID NOs:475-487)

FIG. 2 shows a superset of test sets designed for analysis of PKC specificity from P−4 to P+3. (SEQ ID NOs:488-553)

FIG. 3 provides counts per minute for in vitro phosphorylation by PKC-theta of a superset of peptide pools designed for analysis of PKC specificity from P−4 to P+3 for peptide pools shown in FIG. 2.

FIG. 4 provides Ratio-to-Mean values for different amino acid residues at different positions when using PKC-theta for peptide pools shown in FIG. 2.

FIG. 5 provides a position-specific scoring matrix for PKC-theta using the Log₂ Score for peptide pools shown in FIG. 2.

FIG. 6 provides sequences of a superset of degenerate peptides designed to extend analysis of PKC specificity. (SEQ ID NOs:554-631)

FIG. 7 provides a position-specific scoring matrix for extended positions using PKC-theta for peptide pools shown in FIG. 6.

FIG. 8 illustrates the differences between the previously available Sequence Logo for PKC (left) and a PSSM Logo of the invention for PKC-theta (right).

FIG. 9 illustrates a validation study testing our predictions for PKC-theta and the previously available Scansite prediction for PKC-delta against results for PKC-delta. The results indicate that the predictions made according to the invention are valid and are better than the previously available Scansite method.

FIG. 10 compares the sensitivity and specificity of the present methods with those provided by a previously available Scansite method using PKC-delta as the kinase.

FIG. 11 illustrates validation of the PKC-theta PSSM with a second set of proteomic peptides that were chosen for synthesis/testing based on prior knowledge of PSSM percentiles. Panel A shows results for individual peptides. Panel B shows average results for groups of peptides grouped by PSSM percentile predictions

FIG. 12 illustrates core sequences of a superset of test sets with 1 anchor. position, represented by the formula d??R??S????d. Because of the importance of ‘R’ at P−3 to many basophilic kinases, these test sets are particularly useful for such basophilic kinases.

FIG. 13 illustrates PSSM Logo for results of analysis of the kinase AKT1 with the d??R??S????d superset.

FIG. 14 illustrates proposed abundances of residues for use in degenerate positions.

FIG. 15: Detection of specific phosphorylation of SHP-1 by Western blot with pPKC antibody which is augmented following stimulation by the T-cell receptor.

FIG. 16 illustrates that the sites predicted to be the best PKC sites are also the ones for which the corresponding phosphorylated peptide binds best to the phosphoantibody.

FIG. 17 illustrates that scores derived from different test sets tested at different times are reproducible and scores extrapolated for untested residues can be adequate.

FIG. 18 illustrates how a peptide can be scored using the methods of the invention. (SEQ ID NO:474)

FIG. 19 shows the distribution of scores observed when all Ser/Thr containing sites in 15651 human proteins were scored with the PKC-theta PSSM and shows the cutoffs for scores corresponding to particular low percentile scores.

FIG. 20 illustrates that the PKC site prediction algorithm provided by the invention correctly predicts previously known sites in the MARCKS protein. (SEQ ID NOs:632-640)

FIG. 21 High similarity in specificity between novel and classical PKC isoforms, but atypical PKC differs more and great divergence seen with AKT1 and PKA.

FIG. 22 illustrates the differences between PSSM Logos of different kinases analyzed with the same peptide supersets.

FIG. 23 illustrates validation studies that demonstrate that the predictions made for PKC-zeta are valid and are better predictions for PKC-zeta than for PKC-delta.

FIG. 24 illustrates scoring changes in peptides that are less phosphorylated by PKC-zeta than by PKC-delta.

FIG. 25 illustrates position-specific residue preferences for PKA and PKG determined using the PKC superset.

FIG. 26 illustrates the differences between PSSM Logos of different mutant kinases derived from PKC-theta analyzed with the same peptide supersets. A PSSM Logo for wild type kinase analyzed using low levels of ATP is shown in the lower right corner.

FIG. 27 illustrates the detailed changes in amino acid preferences observed with PKC-theta mutant constructs and with altered kinase assay conditions.

FIG. 28 illustrates that details of residue references for PKC-theta depend on the choices made for anchor and phosphorylation residues in the test sets used.

FIG. 29 illustrates results for ROK-alpha with test sets based on ??R??T???? with only 4 query residues.

FIG. 30 illustrates details of the R-Pair Anchor optimization set.

FIG. 31 illustrates results for analysis of PKA with the R-Pair set shown in FIG. 30.

FIG. 32: shows that the R-Pair set reveals positions associated with the strongest preference for R.

FIG. 33 shows detection of specific phosphorylation of LIMK-2 by Western blot with the pPKC antibody which is augmented following stimulation by the T-cell receptor.

FIG. 34 shows detection of phosphorylation of MLK3 by Western blot with the pPKC antibody.

FIG. 35 is a diagram of a computerized system in conjunction with which embodiments of the invention may be implemented.

### DETAILED DESCRIPTION OF THE INVENTION

The invention relates to determination of the specificity of protein kinases, to visual representation of specificity of kinases, to prediction of sites on sequenced proteins that are most likely to be phosphorylated by each kinase studied, to validation that peptides corresponding to those predicted sites are indeed phosphorylated in vitro by each kinase studied, and to validation of phosphorylation of those sites in vivo.

The term “kinase” (or “protein kinase”) as used herein is intended to include all enzymes that add a phosphate group to an amino acid residue within a protein or peptide. Kinases that may be used in the methods of the invention include protein-serine/threonine specific protein kinases, protein-tyrosine specific kinases and dual-specificity kinase. Other kinases that can be used in the method of the invention include protein-cysteine specific kinases, protein-histidine specific kinases, protein-lysine specific kinases, protein-aspartic acid specific kinases and protein-glutamic acid specific kinases.

A kinase used in the method of the invention can be a wild type or mutant kinase. The kinases employed can be purified native kinases, for example, a kinase purified from its native biological source. Kinases employed can be from a variety of species. Some kinases that can be employed are commercially available (e.g., protein kinase A from Sigma Chemical Co.). Alternatively, a kinase used in the method of the invention can be a kinase produced by creation of a nucleic acid construct and preparing the protein product expressed in vitro or in whole cells (i.e., a “recombinantly produced kinase”). Many kinases have been molecularly cloned and characterized and thus can be expressed recombinantly by standard techniques. Hence, any recombinantly produced kinase that retains its kinase function can be used in the methods of the invention. If the recombinant kinase to be examined is a eukaryotic kinase, it is generally preferable that the kinase be recombinantly expressed in a eukaryotic expression system to ensure proper post-translational modification of the protein kinase. Many eukaryotic expression systems (e.g., baculovirus and yeast expression systems) are known in the art and standard procedures can be used to express a kinase recombinantly. A recombinantly produced kinase can also be a fusion protein (i.e., composed of the kinase and a second protein or peptide) as long as the fusion protein retains the catalytic activity of the non-fused form of the kinase. Furthermore, the term “kinase” is intended to include portions of native protein kinases that retain catalytic activity. For example, a subunit of a multi-subunit kinase that contains the catalytic domain of the kinase can be used in the methods of the invention.

One of skill in the art frequently uses a formula such as the following (I) to represent the amino acid positions within a peptidyl site that may be phosphorylated by a kinase:
(P−4)-(P−3)-(P−2)-(P−1)-P0-(P+1)-(P+2)-(P+3)-(P+4) I
where P0 is the phosphorylated position, P−1 is the amino acid position immediately to the N-terminal side of P0, P+1 is the amino acid position immediately to the C-terminal side of P0, P−2 is the amino acid position that is two residues from P0 on the N-terminal side of P0, etc. This terminology will be used herein as a general description of a kinase phosphorylation site and the variables P−4, P−3 etc. will be used to refer to a particular amino acid position within a kinase phosphorylation site.

In general, key positions that determine kinase specificity are within about four amino acids of the phosphorylated amino acid. However, positions farther than four positions from the phosphorylation site can influence the specificity of a kinase and can be characterized by the methods of the invention.

When one or more positions of a particular peptidyl sequence are determined, a one letter amino acid symbol may be used herein to indicate what amino acid is present at that determined position. The standard three-letter and one-letter abbreviations for amino acids provided in Table 1 are used throughout the application.

TABLE 1

| Amino acid | 3-Letter | 1-Letter |
|---|---|---|
| Alanine | Ala | A |
| Arginine | Arg | R |
| Aspartic acid | Asp | D |
| Asparagine | Asn | N |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The P0 position is the position that can be phosphorylated (the “phosphorylatable position”) and is generally either a serine (S), threonine (T) or a tyrosine (Y) for human kinases. Hence, specific peptidyl sequences generally discussed herein will often have S, T or Y at the P0 position. When any of a defined set of amino acids is present at a given position, for example, when a degenerate mixture of amino acids is used during synthesis of a peptide at that position, a lower case “d” is used herein to represent the degeneracy of that position. To represent peptides in which a residue is phosphorylated, a lower case ‘p’ is used before the residue abbreviation; thus, pS or pSer represents a phosphorylated serine residue, pT or pThr represents a phosphorylated threonine, and pY or pTyr represents a phosphorylated tyrosine.

Design of Single Peptide Test Sets:

The invention provides for determination of the specificity of protein kinases by synthesis of test sets (and supersets) of peptides, subjecting the test sets (or supersets) to phosphorylation by a kinase of interest, and quantifying and analyzing the results.

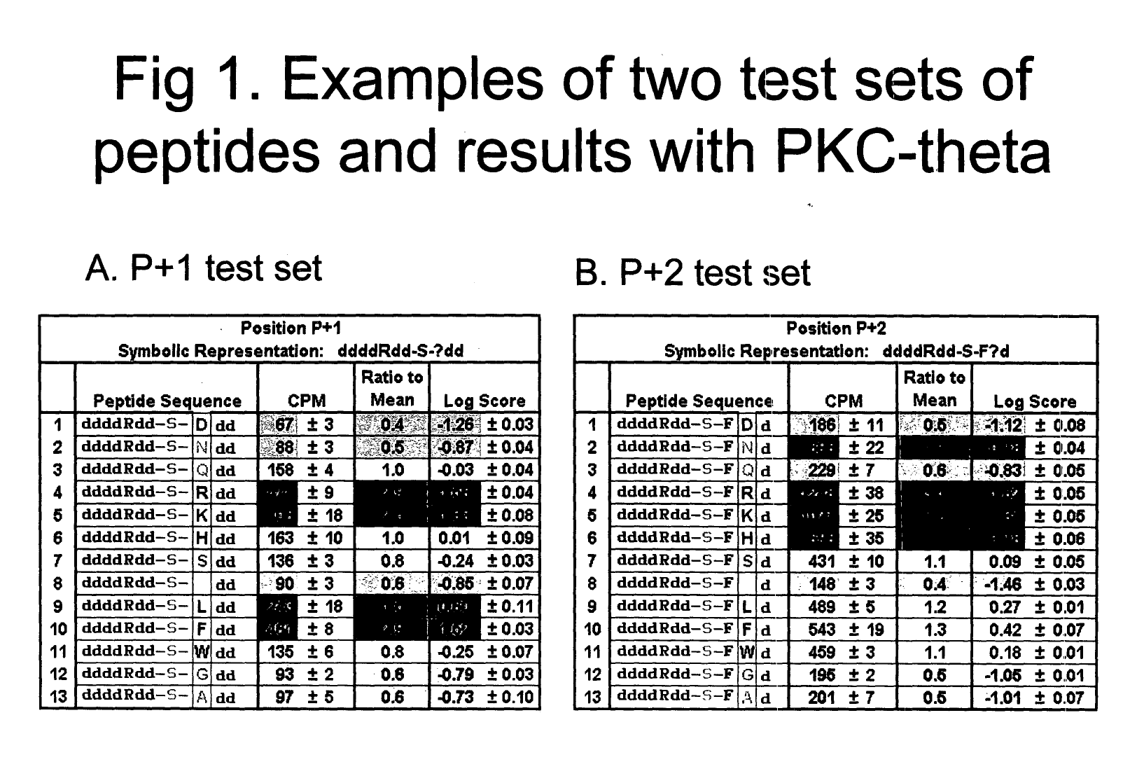

Two simplified embodiments shown in FIG. 1 are used as examples of the methods provided herein. FIG. 1A shows one test set of peptide pools (a “P+1” test set) and FIG. 1B shows a second test set (a “P+2” test set). As used herein, the name of a test set generally identifies which position is being systematically varied (i.e., which position is the “query” position. Each peptide of the two test sets illustrated in FIG. 1 has a “core” sequence comprised of eleven amino acid residues. The term “core” is used to refer to amino acid sequences that play a key role in determining kinase specificity and is used to distinguish such key amino acids from N-terminal or C-terminal residues that are incorporated to provide functions unrelated to determination of specificity (such as for capture of the peptide onto a solid support or for quantification).

Four different types of amino acid positions can occupy the core positions in each of these peptides, as well as the other peptides described herein. These different types of amino acid positions are described below.

1) A phosphorylatable amino acid position is a position occupied by an amino acid to which a phosphate group can be added by a kinase. In eukaryotes S, T, and Y are the primary phosphorylatable residues. However, in other species residues such as histidine are also subject to phosphorylation. This residue occupies the P0 position in each peptide pool in a test set. Hyphens (-) may be used herein around the amino symbol in the P0 position (e.g. -S-) to visually highlight this position. Note that the position of other types of amino acid position in the core sequence are fixed relative to this P0 phosphorylatable position in for all peptide pools in a given test set, and that each amino acid position is expressed relative to the P0 position.

2) An anchor amino acid position is a position in addition to the phosphorylatable amino acid position having a determined amino acid that does NOT vary from one peptide pool to another in the test set. More than one anchor amino acid position can be present in a test set. The location of the anchor amino acid positions and identity of the anchor amino acids at each anchor position are identical for all peptides pools in the test set. For example in the P+1 set shown in FIG. 1A, there is one anchor amino acid: an arginine (R) at position P−3. In the P+2 set, there are two anchor amino acids: an arginine (R) at P−3, and a phenylalanine (F) at P+1. The function of the anchor amino acid positions is to provide sufficient favorable interaction between substrate and kinase to permit measurable phosphorylation of each peptide pool. An anchor amino acid is represented by a single letter amino acid code for the amino acid in that anchor position.

3) A query amino acid position (or a varied position) is a position that is being tested for its effect upon substrate phosphorylation. The symbol “?” is often used herein as a symbol for identifying the query position. Unlike anchor amino acid positions, there is generally only a single query amino acid position within all peptide pools of a test set. In general, a query amino acid is determined (i.e. not degenerate) for a particular peptide pool. However, the query amino acid at that query position is systematically varied from peptide pool to peptide pool within a test set of peptides. Hence, in contrast to the anchor positions, the query or varied position is occupied by different residues within the different peptide pools of a test set. The query or varied position is boxed in red in FIG. 1. The function of the query or varied positions is to allow assessment of the contribution of different amino acids to kinase specificity by determining how each of the different tested amino acids influences the amount of phosphorylation.

4) A degenerate position contains an undetermined amino acid selected from a defined mixture of amino acids. More than one degenerate position is typically present in a test set of peptide pools. For any given peptide pool in a test set, all core positions that are not anchor, phosphorylatable or query positions are degenerate positions. Thus, the presence of one or more degenerate positions means that each peptide pool in a test set of peptides is actually a complex mixture (or “library” of distinct peptides). Although each peptide pool consists of many individual peptides, that peptide pool is often referred to herein as a “peptide,” in keeping with common usage in the literature. Measuring phosphorylation of each such peptide pool assures that the assay reflects the average behavior of a large number of individual sequences. The symbol “d” is used herein as symbol of a degenerate position in the test sets of peptide pools provided herein.

In some embodiments, the query position is not adjacent to an anchor position within the test sets provided herein. In other embodiments, the query position is not adjacent to the phosphorylatable position.

FIG. 1 illustrates the symbolic representation of two test sets of peptides designed for analysis of PKC specificity, and the corresponding peptides pools synthesized for those test sets. The formula ddddRdd-S-?-dd describes the P+1 test set of peptides shown in FIG. 1, where serine is in the P0 position, the query position is P+1, arginine is the anchor amino acid chosen for an anchor position at P−3 and the remaining amino positions are degenerate. Similarly, the formula ddddRdd-S-F-?-d describes the P+2 test set of peptides shown in FIG. 1, where: serine is in the P0 position, the query position is P+2; arginine is the anchor amino acid chosen for an anchor position at P−3; phenylalanine is an anchor amino acid chosen for a second anchor position at P+1; and the remaining amino acid positions are degenerate (d).

Each test set in the embodiments shown in FIG. 1 consists of 13 peptide pools. The residue present at the query position in each peptide pool in a test set is systematically varied. However, the fixed anchor positions within all peptides pools of the test set provide at least a minimal level of kinase recognition and phosphorylation for each peptide in the test set. At the remaining core positions, an amino acid selected from a degenerate mixture of amino acids is used.

Analysis of Kinase Specificity by Phosphorylation of Test Sets

Determination of kinase specificity is made by phosphorylating the test sets of peptides with a kinase of interest. Methods of the invention for determining the substrate specificity of a kinase generally involve contacting each peptide pool in at least one test set of peptide pools with a kinase and a ?-labeled ATP, quantifying the amount of label incorporated into each peptide pool, and comparing the quantity of label incorporated into a peptide pool with the quantity of label incorporated into at least one other peptide pool.

Hence, a test set of peptides is synthesized, for example, the P+1 test set having the thirteen sequences shown in FIG. 1 panel A. The synthesized peptide pools in the test set are reconstituted to standardized concentrations, and replicate samples of the peptide pools are contacted with a kinase under assay conditions that permit phosphorylation at the P0 position. The amount of phosphorylation of each peptide pool can be determined, for example, by observing the radioactivity incorporated into the peptide pool after using ?³²P-ATP as a donor of the phosphate group during the phosphorylation assay.

FIG. 1 panel A provides results of such a phosphorylation assay for the P+1 test set of peptides. The “raw data” are measured as counts per minute (cpm). As shown in FIG. 1, marked variation exists in the amount of phosphorylation present in different peptide pools of a test set, reflecting important contributions of the single residue by which they differ. Furthermore, the SEMs (standard error of the mean of replicate values) are small, indicative of good assay agreement between replicates.

In some embodiments, the determination of residue preference is made by comparing the cpm incorporated into each peptide, with the geometric mean cpm incorporated for all the peptides in the set. That ratio is shown in FIG. 1 within the column labeled ‘Ratio-to-Mean.’ The Ratio-to-Mean is also referred to herein as residue preference. A Ratio-to-Mean greater than 1.0 indicates that the selected query residue in the corresponding peptide is preferred by the kinase over the other types of query residues tested. For example, a Ratio-to-Mean of 2.9 was observed for ‘F’ in the P+1 test set, indicating that phenylalanine at P+1 is highly preferred by the kinase used for this assay (PKC-theta). A ratio less than 1.0 indicates that the selected query residue in the corresponding peptide pool is disfavored compared to the other residues tested. For example, a ratio of 0.4 was obtained for ‘D’ in the P+1 test set, indicating that aspartic acid at P+1 is disfavored by the kinase used for this assay. To visually emphasize the preferred residues, the data in FIG. 1 have been shaded in red to indicate favored residues with residue preferences greater than 1.5. In contrast, data relating to disfavored residues have been shaded in blue, indicating that the residue preference is less than 0.67 (i.e. 1.0 divided by 1.5).

A value called ‘Log Score’ was calculated for each residue by determining the log (base 2) of the Ratio-to-Mean. As a result of this mathematical transformation, favored residues have a positive score, and disfavored residues have a negative score. This score obviously differs depending on the position of the residue in the peptide (compare the P+1 test set in FIG. 1 panel A with the P+2 test set in FIG. 1 panel B). Hence, each value represents a position-specific score for a particular amino acid residue. As indicated in FIG. 1 panel A, arginine, lysine, phenylalanine and leucine are preferred residues at the P+1 position for the kinase tested (PKC-theta). In contrast, aspartic acid, asparagine, proline, glycine and alanine are disfavored at the P+1 position for the kinase tested (PKC-theta).

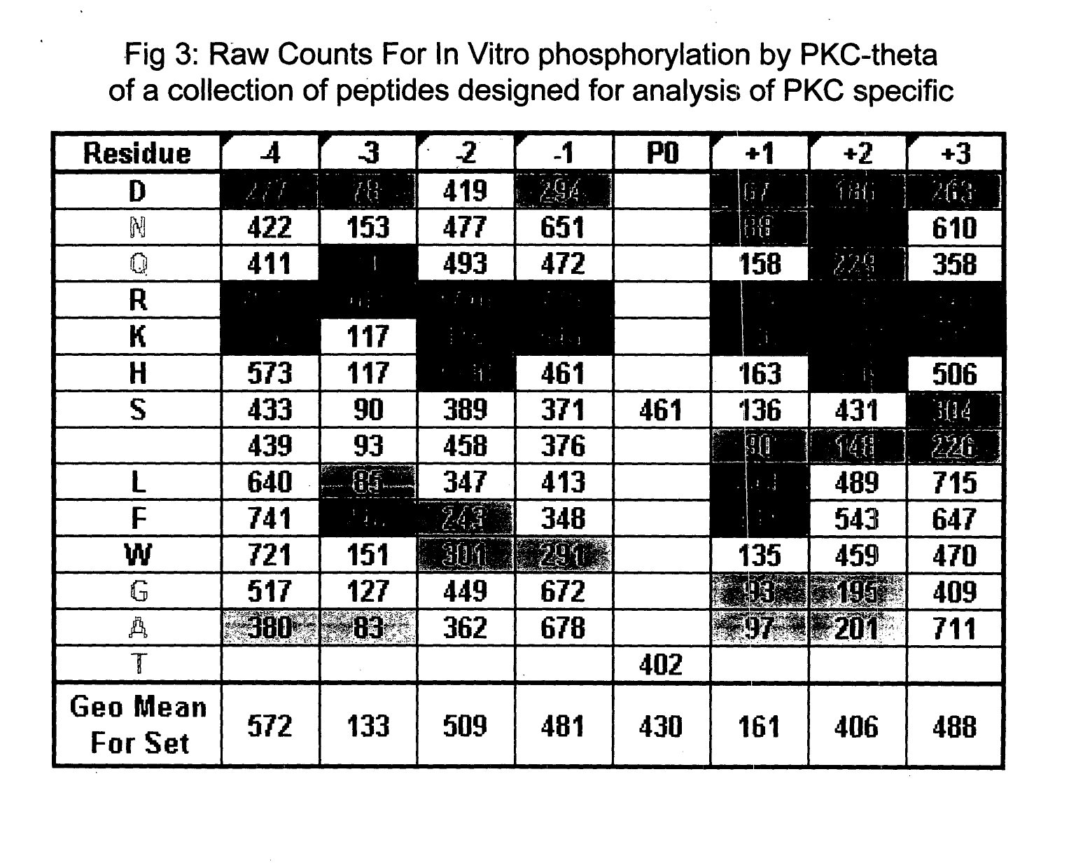

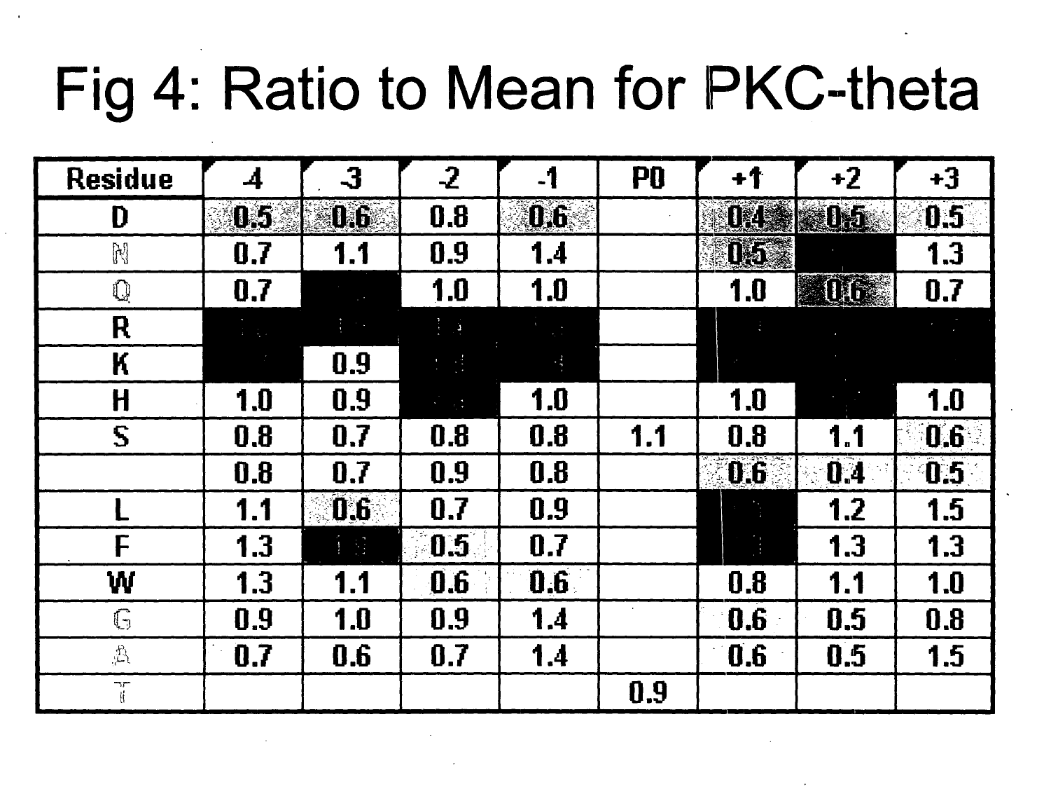

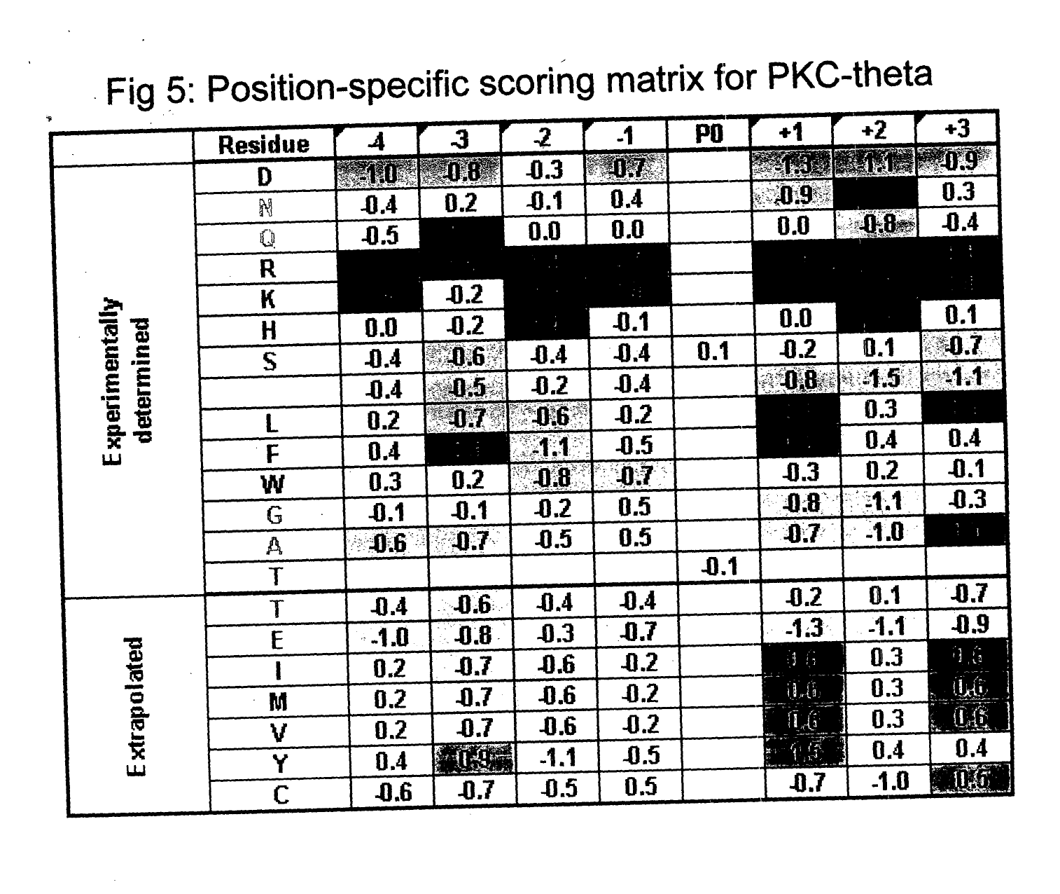

The invention provides computer-executable instructions for performing the calculations described above. One preferred embodiment uses software tools enabled by use of a spreadsheet application such as Microsoft Excel running on operating system such as Windows 2000 on a hardware platform such as a Dell Latitude using a microprocessor such as an Intel Pentium chip. For example, a spreadsheet is customized for a given superset of test peptides; manipulation of that data is provided by formulas embedded in that spreadsheets. Output of counts per minute from TopCount NXT Microplate Scintillation and Luminescence Counter in 96 well plate format are input into the spreadsheet. The results are displayed to the user in the spreadsheet; FIG. 3, FIG. 4. and FIG. 5 are screen captures from such a spreadsheet. In one embodiment additional processing of data is provided by automation of additional functions in the spreadsheet using the language Visual Basic for Applications, which is embedded in the Excel application; in other embodiments additional automation is provided by software objects exposed by the Excel interface and manipulated by software external to Excel, such as Microsoft Visual Basic. This embodiment uses this same computational infrastructure for performing the manipulations described in Example 3.

Thus, the invention provides a computer readable medium having computer-executable instructions for determining quantitative values describing the preference of a kinase for a defined amino acid at a defined substrate position wherein the input data comprises experimental data on phosphorylation of a test set of peptides comprising at least two peptide pools, wherein every peptide in each of the peptide pools comprises one phosphorylatable amino acid position, one query amino acid position, wherein each peptide of every peptide pool has an identical phosphorylatable amino acid that can be phosphorylated by a kinase at the phosphorylatable amino acid position and the query amino acid position is at a defined position relative to the phosphorylatable amino acid position within every peptide of every peptide pool but a query amino acid's identity at the query amino acid position is systematically varied from one peptide pool to the next peptide pool within the test set of peptide pools

Supersets Constructed from Multiple Test Sets

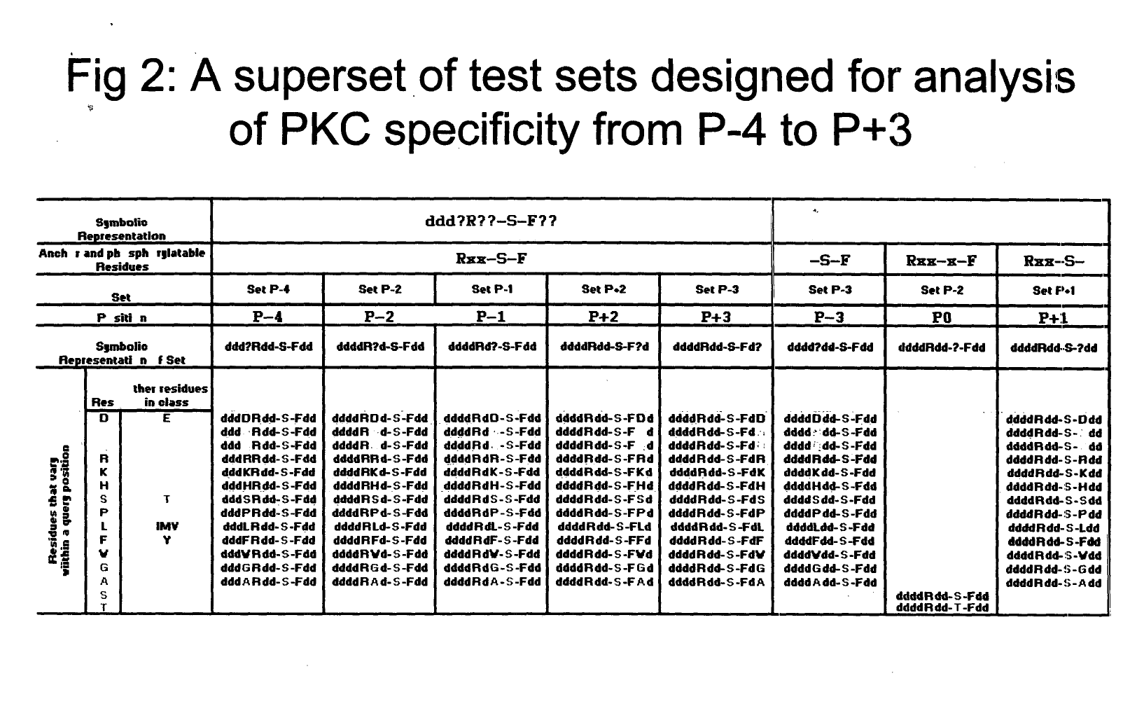

The test sets illustrated in FIG. 1 provide information on positions P+1 and P+2, based on the location of the query position relative to the phosphorylatable anchor residue. In general, all positions within a test substrate can separately be made into query positions by constructing a test set of peptides for each query position. Hence, one of skill in the art can make, for example, P−7, P−6, P−5, P−4, P−3, P−2, P−1, P+1, P+2, P+3, P+4, P+5, P+6 and P+7 test sets of peptides and systematically vary the type of amino acid at each of these query positions. Such a large of test sets of peptide pools with query residues at substantially all the different positions is referred to as a superset. In some embodiments, each position close to the phosphorylation site (P0) will be a query position and the appropriate test sets of peptides within the superset will be made and tested to ascertain which amino acid is preferred by the kinase at those query positions. FIG. 2 shows such a superset of test sets of peptides designed and synthesized to test the specificity of PKC and related kinases at all query positions from P−4 to P+3. This superset includes the two test sets shown in FIG. 1 together with six other test sets.

Such supersets are phosphorylated by a kinase of interest as described for the test sets above. FIG. 3 shows the raw data (cpm) obtained for a representative experiment testing PKC-theta on the superset shown in FIG. 2. FIG. 4 shows the Ratio-to-Mean for that data, calculated as described above. FIG. 5 shows the Log (base 2) score for that data, calculated as described above. Taken together, the scores derived from analysis of a superset of peptides (e.g. FIG. 5) constitute a position-specific scoring matrix (PSSM) describing the residue preference of the selected kinase at different positions around the phosphorylation site.

A reduced set of amino acid residues can be used in the query position of the test sets of peptides. Experimental data obtained for such reduced sets of query amino acids do not provide information for all naturally occurring residues. In some embodiments, data that is not obtained experimentally can be estimated from existing data. For example, the lower boxed region shown in FIG. 5 provides extrapolated data for residues that were not tested, but that have similar physico-chemical properties to the peptides tested. Thus, in this case data for glutamic acid (E) was inferred from aspartic acid (D), data for isoleucine (I), methionine (M) and valine (V) was inferred from leucine (L), data for tyrosine (Y) was inferred from phenylalanine (F). Where cysteine was excluded from the residues analyzed, a score for cysteine was likewise created from scores for other residues. Such extrapolation can be accomplished in a variety ways, for example, by assigning a score of zero, or assigning the score corresponding to other residues such as alanine. The accuracy of these extrapolated scores can then be tested as described below (Example 2)

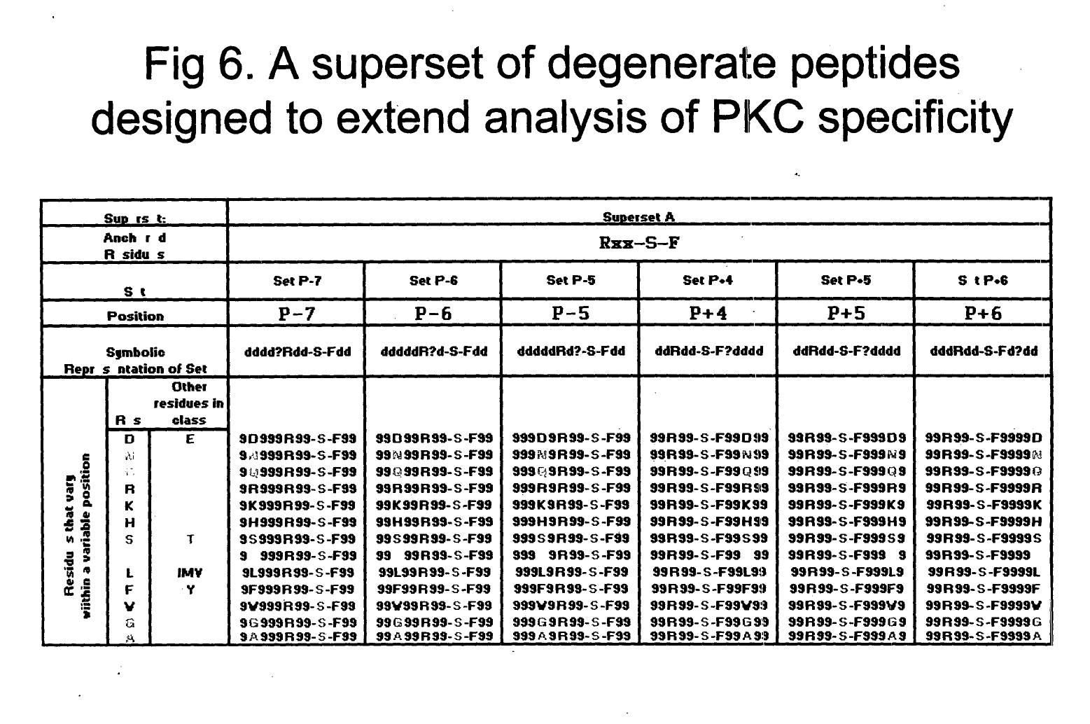

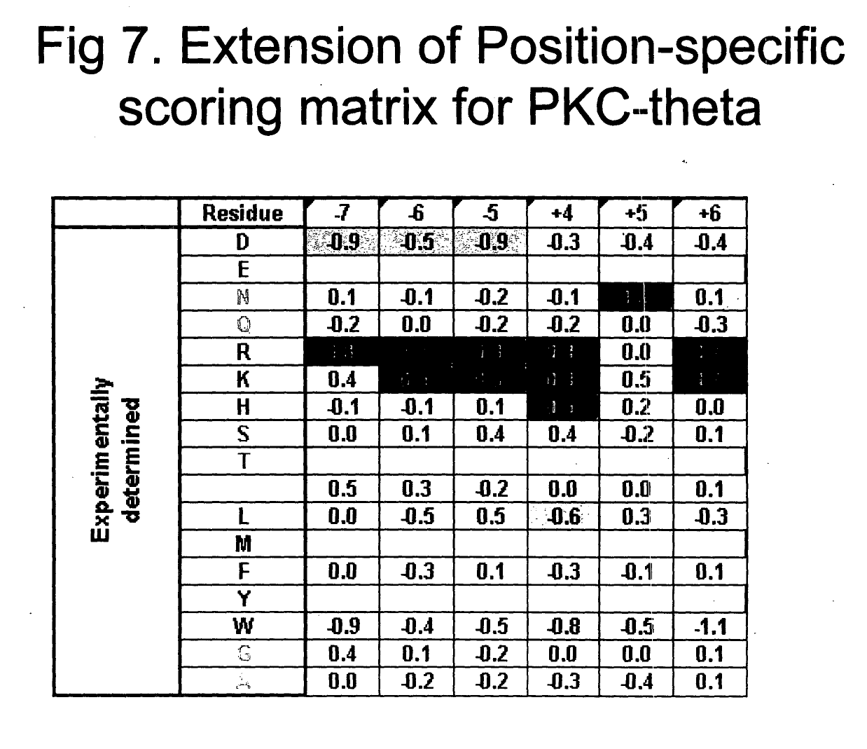

The method of the invention is flexible so that greater or lesser numbers of test sets can be included for testing as many positions as desired. For example, FIG. 6 lists the sequences of a superset of peptide pools designed to extend the analysis of PKC specificity to include positions P−7 through P−5 and P+4 thru P+6. FIG. 7 shows an extended position-specific scoring matrix for positions P−7 through P−5 and P+4 through P+6 derived from testing PKC-theta with the test sets shown in FIG. 6. Taken together, the scores from FIG. 5 and FIG. 7 provide a position-specific scoring matrix for PKC-theta for positions P−7 to P+6. The ability to combine results from different sets and different experiments is a convenient aspect of the invention.

Visual Representation of Kinase Specificity

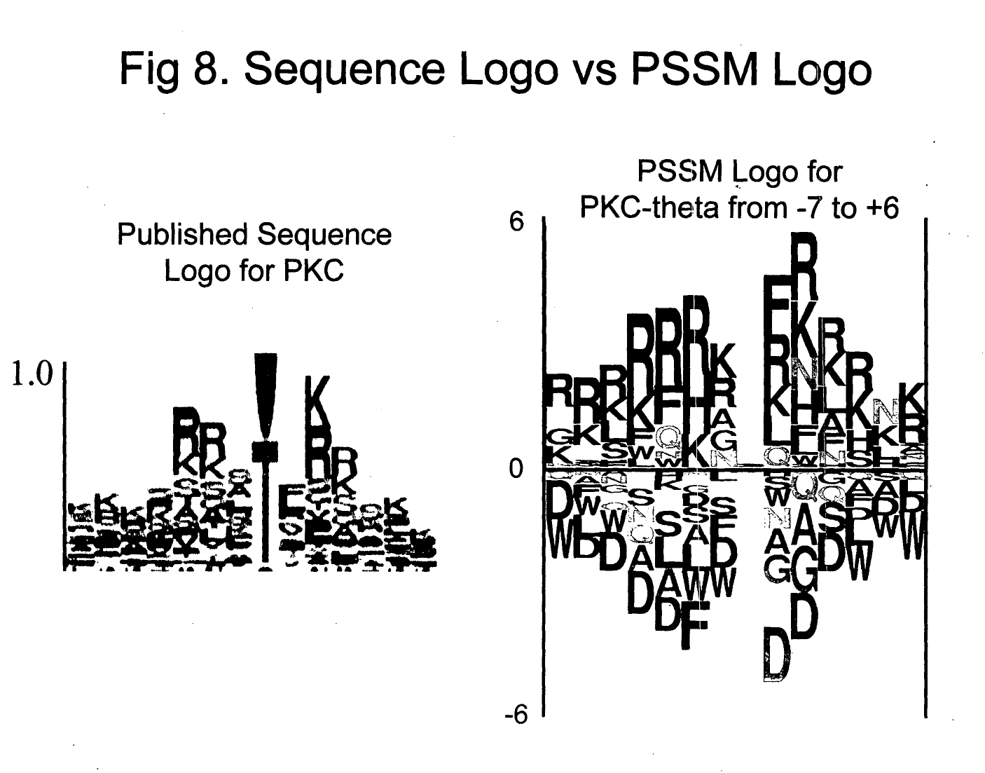

An efficient strategy for visual representation of specificity information is important for conceptualizing and communicating findings on kinase specificity. A previously described method for visualizing peptide specificity data is via the Sequence Logo developed by Thomas Schneider (Schneider T D et al. 1990. Nucleic Acids Res. 18:6097-6100). In that article, the method is described as follows “The height of each letter is made proportional to its frequency, and the letters are sorted so the most common one is on top. The height of the entire stack is then adjusted to signify the information content of the sequences at that position.” This visualization method is illustrated on the left side of FIG. 8 for a published Sequence Logo generated by the Schneider method for protein Kinase C (PKC) (Kreegipuu A et al. 1998. FEBS Lett 430:45-50).

The invention provides a new method for visualizing which amino acids are preferred in the substrate of a kinase. This method involves use of a position specific residue scoring matrix (PSSM) to generate a PSSM Logo. Each position in a PSSM is represented in a PSSM Logo by a vertical stack of amino acid residue single letter codes. The height of each code is made proportional to the absolute value of a Log Score, and the positions of the codes in the stack are sorted from bottom to top in ascending value by the quantitative parameter. An example of a PSSM Logo of the invention is provided on the right side of FIG. 8, which illustrates the results for analysis of PKC-theta with peptide pools shown in FIG. 2 and FIG. 6.

Two major differences exist between the previously available Sequence Logo and a PSSM Logo of the invention. The most fundamental difference between a Sequence Logo and a PSSM Logo is that the PSSM Logo visually emphasizes the residues that are disfavored by the kinase as well as the ones that are favored by the kinase. In contrast, the Sequence Logo only emphasizes the residues that are favored. Such distinction is not a trivial distinction, but rather represents a fundamental difference in emphasis between the method of the invention and those of prior workers. In particular, the present methods accurately determine which amino acid residues are disfavored, which has not previously been emphasized and which can be a controlling factor in determining kinase specificity (see below).

A secondary difference between the previously available Sequence Logo and a PSSM Logo of the invention is in the parameters represented by the PSSM Logo versus those represented by the Sequence Logo. The Sequence Logo, as described by Schneider, is determined by a combination of the parameters referred to as ‘information content’ of that position, and of the residue frequency. In contrast, in a preferred embodiment, the PSSM Logo reflects the log scores obtained by the methods of the invention, which are not interchangeable with residue frequency. In other embodiments, the parameter represented in the PSSM Logo is the log of the ratio of [residue frequency]/[control residue frequency]. Hence, the PSSM Logo is distinct from the Sequence Logo.

Note that use of a PSSM Logo is not restricted to findings of kinase specificity, but rather is generally useful for expressing results pertaining to amino acid residue preference. Thus, for example, results of other experimental methods for determination of residue preference for peptide binding (rather than phosphorylation) can equally well be represented with a PSSM Logo. Moreover, nucleotide sequence preferences can also be represented using a PSSM Logo.

One embodiment uses software tools enabled by use of a spreadsheet application such as Microsoft Excel running on operating system such as Windows 2000 on a hardware platform such as a Dell Latitude using a microprocessor such as an Intel Pentium chip. Software objects exposed by the Excel interface are manipulated by software external to Excel, such as Microsoft Visual Basic. Information in the spreadsheet for each substrate position consists of paired columns, one comprising the residue code and one comprising the log2 scores. Rows in that pair of columns are sorted in descending order by log2 scores. That sorted information is converted into a file of commands using postscript programming language which instruct a postscript printer (such as as Xerox Phaser 6200 printer) to create symbols of the appropriate size and position in a column. Successive columns in the PSSM are processed similarly and the postscript code instructs the printer to move horizontally to position information on each successive substrate position into adjacent columns.

Thus, the invention provides a computer readable medium having computer-executable instructions for performing a method of visually displaying amino acid or nucleotide sequence preferences, the method comprising: representing a position in a peptide or a nucleic acid sequence with a stack of single letter symbols for amino acids or nucleotides; and displaying one or more stacks of letters wherein each symbol's height is proportional to the absolute value of a quantitative parameter that is positive for favored amino acids or nucleotides and negative for disfavored amino acids or nucleotides and wherein each symbol's position within the stack is sorted from bottom to top in ascending value by the quantitative parameter.

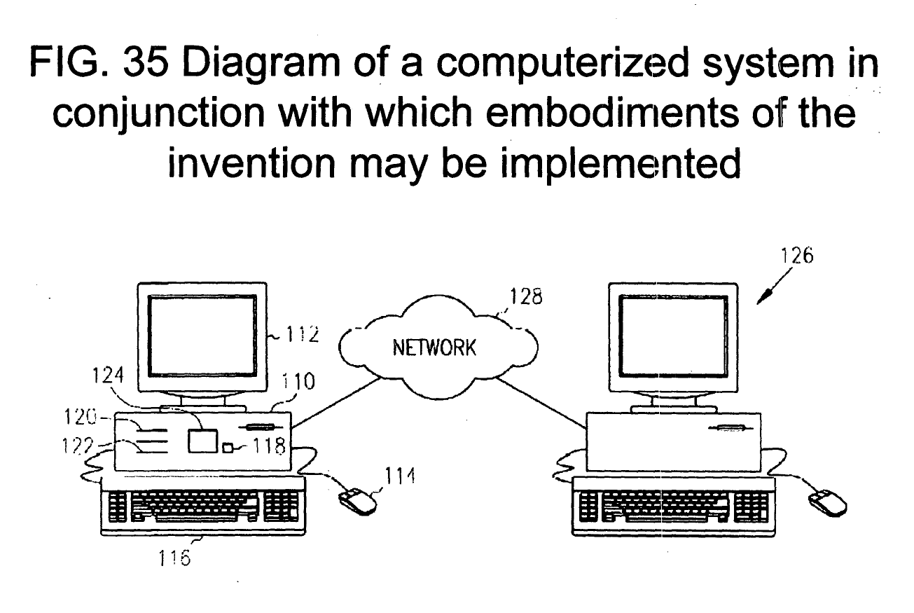

The invention also provides an overview of the hardware and the operating environment in conjunction with which embodiments of the invention can be practiced. FIG. 35 is a diagram of a computerized system in conjunction with which embodiments of the invention may be implemented. Thus, in one embodiment, computer 110 is operatively coupled to a monitor 112, a pointing device 114 and a keyboard 116. Computer 110 includes a central processing unit 118, random-access memory (RAM) 120, read-only memory (ROM) 122, and one or more storage devices 124, such as a hard disk drive, a floppy disk drive, a compact disk read-only memory (CD-ROM), an optical disk drive, a tape cartridge drive or the like. RAM 120 and ROM 122 are collectively referred to as the memory of computer 110. The memory, hard drives, floppy disks, etc., are types of computer-readable media. The computer-readable media provide nonvolatile storage of computer-readable instructions, data structures, program modules and other data for computer 110. The invention is not particularly limited to any type of computer 110.

Monitor 112 permits the display of information for viewing by a user of the computer. Pointing device 114 permits the control of the screen pointer provided by the graphical user interface of window-oriented operating systems such as the Microsoft Windows family of operating systems. Finally, keyboard 116 permits entry of textual information, including commands and data, into computer 110.

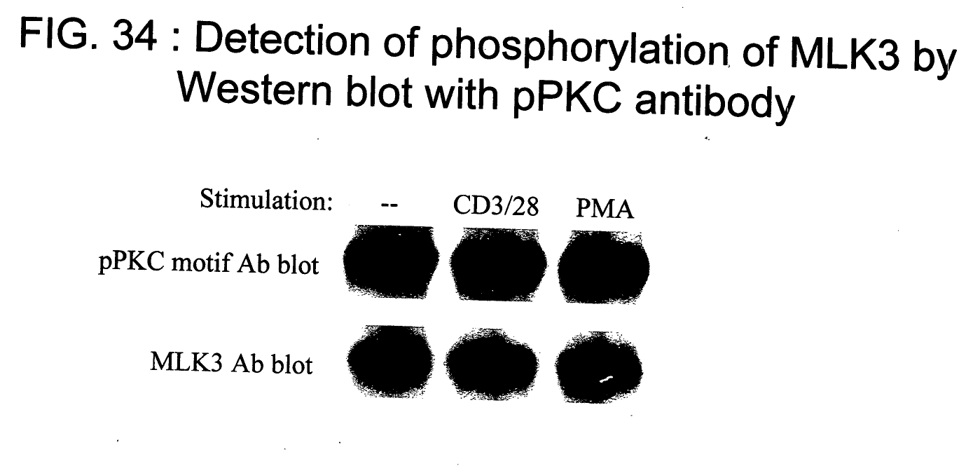

The computer 110 operates as a stand-alone computer system or operates in a networked environment using logical connections to one or more remote computers, such as remote computer 126 connected to computer 110 through network 128. The network 128 depicted in FIG. 34 comprises, for example, a local-area network (LAN) or a wide-area network (WAN). Such networking environments are common in offices, enterprise-wide computer networks, intranets, and the Internet.

An example hardware and operating environment in conjunction with which embodiments of the invention can be practiced has been described.

Validation of the Results Obtained Using the Methods Described

One of the principle uses for the methods of the invention is to predict sites of phosphorylation in proteins whose sequences are known but whose phosphorylation sites are unknown. The ability to correctly predict phosphorylation sites will depend on the correctness of the methods employed. If the values for residue preference in for a kinase are incorrect, then the predictions are unlikely to be correct. As described herein a PSSM generated by the methods of the invention will generally provide better and more complete substrate specificity information than previously employed methods and predictions employed.

Rather surprisingly, systematic validation has not been reported for previously reported predictive algorithms, such as those proposed by U.S. Pat. No. 6,004,757 to Cantley et al. For example, Nishikawa K et al. 1997. J Biol Chem 272:952-960 describes an approach for determining peptide specificity for PKC, but the validation provided was limited to a showing that the optimal peptides predicted for two different kinases are preferentially phosphorylated by their respective kinases. No validation was provided that the sequence identified was the best sequence, or that good in vitro substrates can be identified by using the remainder of the information derived from the technique. While, Cantley and co-workers also propose that the results of such predictions correlate with physiologically relevant sites, such assertions are based on a modest correlation with anecdotal results from the literature.

One approach to validating a substrate identification method can involve, for example, comparison of substrate sites predicted by the method with in vitro phosphorylation results obtained using the selected kinase and peptides of known sequences. Such a systematic validation has been performed for the methods described herein. For example, a panel of seventy five peptides was synthesized, the phosphorylation observed for each peptide was experimentally measured, the amount of phosphorylation was quantified, the phosphorylation results for each peptide were normalized to the phosphorylation observed with the best substrate tested and these amounts were compared with predictions made according to the invention and according to the procedures provided by others. These peptides are referred to herein as proteomic peptides because their sequences are chosen from proteins in the human proteome; unlike the test sets employed herein, these peptides include no degenerate positions

Fairness of a validation strategy requires that the choice of test peptides not be unfairly biased by findings from the PSSM being validated. The choice of the peptides in Table 2 was not biased by information from the PSSM-based scoring illustrated herein because the peptides were chosen and synthesized more than five months before the method was established. The dominant criteria for selection of the peptides was computerized scanning of human protein sequences amongst NCBI reference sequences (see website at ncbi.nlm.nih.gov/) to identify sites with an abundance of positively charged residues in positions P−3 to P+3 relative to a potential P0 phosphorylation position (S or T), and with good diversity in the P−1 and P+1 positions.

The results of this analysis for phosphorylation are provided in Table 2. While the results provided in Table 2 show measured phosphorylation by PKC-delta, the PKC-delta predictions made by the methods of the invention (shown in Table 2) were actually based upon data obtained by PKC-theta. In contrast, data generated by the methods of Cantley and co-workers was available for PKC-delta (Nishikawa K et al. 1997. J Biol Chem 272:952-960; and Scansite at scansite.mit.edu). Because the predictions from the present methods are based on PKC-theta, which is distinct from PKC-delta but is the PKC isoform closest to PKC-delta, the comparison provided in Table 2 is biased in favor of the method provided by Cantley and co-workers. Despite this bias, the results demonstrate that predictions made by the methods of the invention are better than predictions made by the methods of Cantley and co-workers (Scansite).

TABLE 2

Validation of the Present Methods
Comparison of Present
Method vs Scansite Predictions

| | |
|---|---|
| Prediction | Measured |
| (percentile) | in vitro |

| | | | | |
|---|---|---|---|---|
| | | In- | | phos- |
| SEQ | | vention | Scansite | phoryl- |
| ID | | for PKC- | for PKC- | ation by |
| NO: | Sequence | theta | delta | PKC-delta |

| | | | | |
|---|---|---|---|---|
| 1 | HVRRRRGTFKRSKLRARD | 0 | 0.26 | 100 |
| 2 | KKKKRASFKRKSSKKG | 0 | 0.01 | 76 |
| 3 | NRKKKRTSFKRKA | 0.1 | 0.05 | 66 |
| 4 | KFARKSTRRSIRLPE | 0.9 | 4.29 | 52 |
| 5 | RQRKRKLSFRRRTDKD | 0 | 0.35 | 42 |
| 6 | PRLIRRGSKKRPAR | 0 | >5 | 40 |
| 7 | RKIPKRPGSVHRTPSRQ | 0.2 | 4.23 | 38 |
| 8 | AARKKRISVKKKQEQ | 0.2 | 0.04 | 35 |
| 9 | QKKSRLRRRASQLKI | 0.1 | 3.83 | 34 |
| 10 | AQIVKRASLKRGKQ | 0.5 | 0.03 | 32 |
| 11 | KKKFRTPSFLKKSKK | 0.4 | 1.52 | 25 |
| 12 | KKKKKRFSFKKSFKL | 0.2 | 0 | 24 |
| 13 | WKGKRRSKARKKRK | 2.5 | >5 | 22 |
| 14 | EYLERRASRRRAV | 0.1 | >5 | 20 |
| 15 | RGFLRSASLGRRASFHLE | 0 | 0.41 | 18 |
| 16 | DGQKRKKSLRKKLD | 0 | >5 | 17 |
| 17 | AGWRKKTSFRKPKED | 0.2 | 0.75 | 17 |
| 18 | KKRFSFKKSFKLSGFSFKKN | 0.2 | 0.01 | 16 |
| 19 | AGSFKRNSIKKIV | 0.3 | 1.69 | 14 |
| 20 | GAPPRRSSIRNAH | 0.4 | >5 | 13 |
| 21 | KLAVGRHSFSRRSGV | 0.5 | >5 | 12 |
| 22 | LLKKRDSFRTPRDSKLE | 2.5 | 2.51 | 12 |
| 23 | QKRHARVTVKYDRRE | 1.5 | 4.49 | 10 |
| 24 | EKIKRSSLKKVDSLKK | 1.5 | 0.02 | 10 |
| 25 | EILSRRPSYRKILND | 0.1 | >5 | 9 |
| 26 | ALRRPSLRREADD | 0.2 | >5 | 9 |
| 27 | KKRKKKSSKSLAHA | 2.7 | 0.02 | 8 |
| 28 | KRPGKKGSNKRPGKR | 4 | 0.48 | 8 |
| 29 | RKNDRKKRYTVVGNP | >5 | >5 | 8 |
| 30 | KEVVRTDSLKGRRGR | 1.5 | >5 | 7 |
| 31 | RKKRKKKSSKSLAHAGVALA | 2.7 | 0.02 | >5 |
| 32 | KATTKKRTLRKNDRK | 1.7 | 0.48 | >5 |
| 33 | QQKIRKYTMRRLLQE | 0.5 | >5 | >5 |
| 34 | EGGDRRASGRRK | 2.1 | >5 | 5 |
| 35 | GLLDRKGSWKKLDDM | 2.1 | 3.26 | 4 |
| 36 | GENVLKKSMKSRVKG | 5.2 | >5 | 4 |
| 37 | AYIERMNSIHRDLRA | 3.1 | >5 | 3 |
| 38 | NYLRRRLSDSNFMAN | 0.9 | >5 | 3 |
| 39 | LLGSGKVTDRKAL | >5 | >5 | 3 |
| 40 | NMEAKKLSKDRMKKY | >5 | >5 | 3 |
| 41 | FVHQASFKFGQGD | 1.5 | 0.04 | 3 |
| 42 | QPEGLRSLKKPDRKKR | >5 | >5 | 3 |
| 43 | AWVTVHEKKSSRKSEYL | 4.2 | 2.95 | 3 |
| 44 | VLAKKGTSKTPVPE | >5 | 2.43 | 2 |
| 45 | VFREHQRSGSYHVRE | 0.1 | >5 | 2 |
| 46 | GQAWGRQSPRRLED | >5 | >5 | 2 |
| 47 | ARIIGEKSFRRSVVG | 2.7 | 0.69 | 2 |
| 48 | AVNSRRRAGQKKK | 5 | >5 | 2 |
| 49 | VQQLLRSSNRRLEQL | >5 | >5 | 2 |
| 50 | ENLRRVATDRRHLGH | 0.8 | >5 | 2 |
| 51 | DLLGKKVSTKTLSEDD | >5 | 4.05 | 2 |
| 52 | HKHSPEKRGSERKEG | >5 | >5 | 2 |
| 53 | AKNLKTLQKRDSFIG | >5 | 0.41 | 2 |
| 54 | ENLRKVTTDKKSLAY | >5 | 0.01 | 2 |
| 55 | DDMEHKTLKITDFG | 1.5 | >5 | 2 |
| 56 | EARLGAASLKFGARD | >5 | 0.01 | 2 |
| 57 | KNVVKLLSSRRTQDR | >5 | 4.49 | 2 |
| 58 | RVKLGTLRRPEGP | >5 | 4.05 | 1 |
| 59 | PVNKRSKYTMMK | 4.1 | 0.18 | 1 |
| 60 | LRRKHLGTLNFGGIR | >5 | 0 | 1 |
| 61 | VDNILKKSNKKLEEL | 5.3 | >5 | 1 |
| 62 | AVRDMRQTVAVGVIK | >5 | 0.84 | 1 |
| 63 | QRQERIFSKRRGQDF | 3.4 | >5 | 1 |
| 64 | ALRAPKPTLRYFTTERF | >5 | 0 | 1 |
| 65 | IKVTHKATGKVMVMK | >5 | >5 | 1 |
| 66 | GFAKKIGSGQKTWTF | >5 | 0.15 | 1 |
| 67 | AINSRETMFHKERFK | >5 | >5 | 1 |
| 68 | RGEGHKPSIAHRDFK | >5 | >5 | 1 |
| 69 | LALTARESSVRSGGAG | >5 | 0 | 1 |
| 70 | HERKGSDKRGDNQ | 4.1 | >5 | 1 |
| 71 | RRRQKRRTGALVLSRGGKR | >5 | >5 | 1 |
| 72 | LTDPKEDPIYDEPEGLAPVPG | >5 | >5 | 0 |
| 73 | IDYYKKTTNGRLPVK | >5 | >5 | 0 |
| 74 | IDYYKKTSNGRLPVK | >5 | >5 | 0 |
| 75 | EEAEHKATKARLADK | >5 | >5 | 0 |

Two steps are involved in the validation process: making the predictions, then assessing the predictions by comparison with measured values. When a PSSM is obtained by the methods of the invention, the calculation of a prediction is straightforward, using the algorithms described herein (see, e.g., example 3).

Table 2 compares the present predictions with actual measurements of phosphorylation on validating peptides. The method of synthesis of the validating peptides was as described elsewhere in the application, and each included an N-terminal linker sequence of biotinylated-Lys-dansylated-Lys-Pro-Pro-Gly (SEQ ID NO:231). The length of the remaining “core” of the validating peptides ranged from 12-21 residues with one to five S/T residues. In vitro phosphorylation of these validating peptides was measured in the manner described herein. Measurements were obtained by phosphorylation of the validating peptides with PKC-delta at a peptide concentration of 10 nM. In vitro phosphorylation results for the validating peptides were expressed as normalized values, namely as a percentage of phosphorylation of the best validating peptide substrate in the group. Hence, a higher value for the measured in vitro phosphorylation of a validating peptide indicated that the validating peptide was phosphorylated to a greater extent than a validating peptide with a lower phosphorylation value.

Many of the peptides employed (Table 2) have multiple serine/threonine residues; the score for a peptide is determined by scoring each Ser/Thr in the peptide and the lowest (i.e. best) percentile for all residues that could be phosphorylated was taken as the percentile for the peptide.

In addition to the measured value, Table 2 tabulates percentile prediction scores for the validating peptides where the prediction scores were obtained either by the methods of the invention or by the methods of Cantley and co-workers. To obtain predictions made as described by Cantley et al, the sequence of the peptide was analyzed using Scansite (see website at scansite.mit.edu/). Scansite is a website made publicly available by L. Cantley and M. Yaffe to predict best substrates based on data derived by the Cantley degenerate peptide strategy. By both the present methods and by the methods of Cantley, a lower positive prediction value indicated a stronger prediction that the peptide will be phosphorylated. Using the conventions of Scansite, predictive percentile scores greater than 5 were shown as >5.

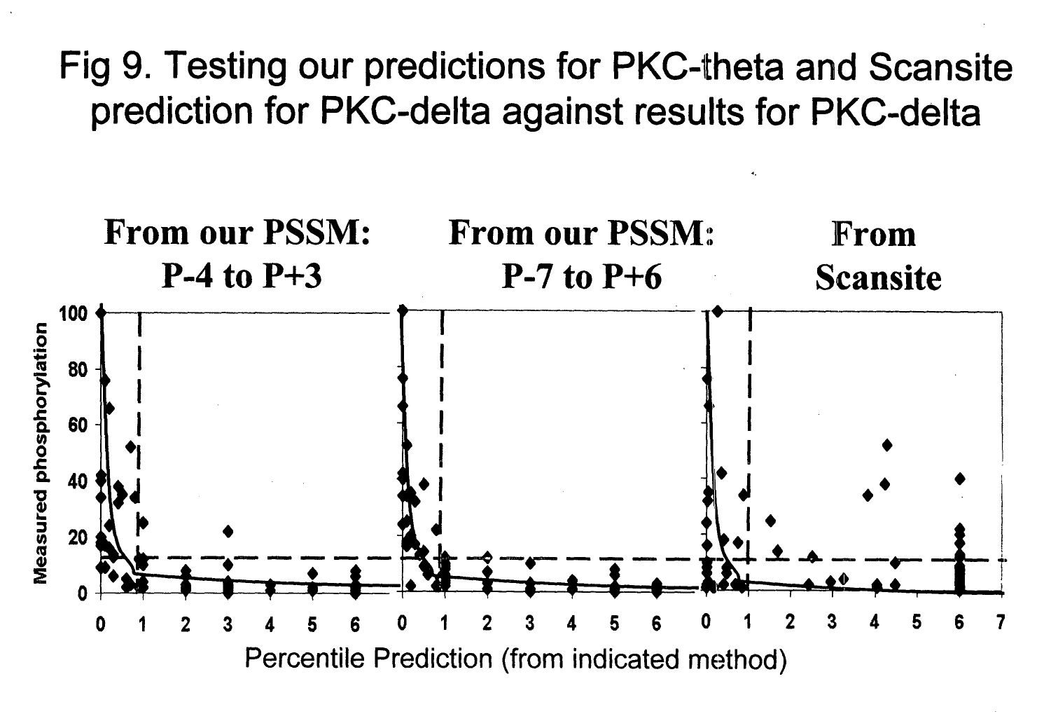

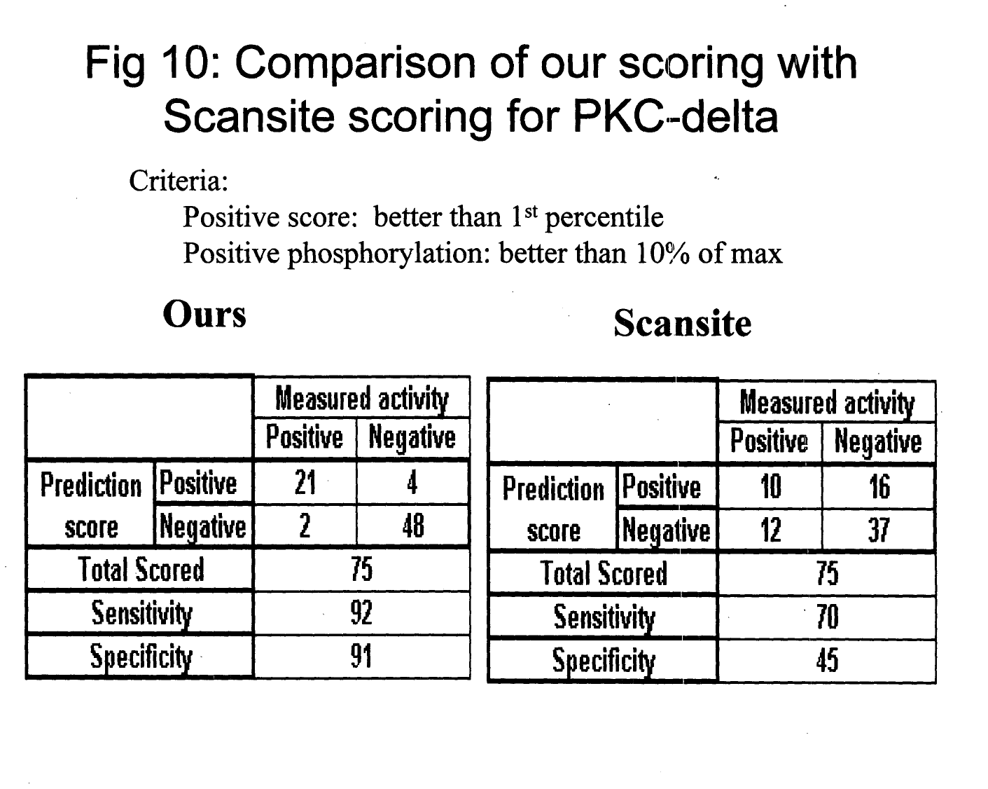

As shown in Table 2, FIG. 9, and FIG. 10, the methods of the invention are better predictors of which peptide sequence will be phosphorylated than are the methods provided by the prior art. For example, peptide SEQ ID NOs: 4, 7, 9 and 11 were highly phosphorylated by the in vitro validating assay but the Scansite methods predicted significantly poorer levels of phosphorylation than did the methods of the invention. Similarly, peptide SEQ ID NOs:60, 64, 66 and 69 were poorly phosphorylated by the in vitro validating assay but the Scansite methods predicted significantly higher levels of phosphorylation than did the methods of the invention.

The predictive accuracies of the methods of the invention and those of Cantley and co-workers (Scansite) are summarized in FIG. 9. FIG. 9 provides a correlation between the predicted percentile and the measured phosphorylation for each peptide. Results are shown for three different predictions: predictions of the invention based only on positions −4 to +3 for PKC-theta; predictions of the invention based on positions −7 to +6 of PKC-theta and the Scansite prediction for PKC-delta. A curve has been overlaid on each of the three plots to indicate what the correlation might be expected to look like. Note that accurate predictions will have few peptides in the upper right (false negatives) or the extreme lower left (false positives). Inspection of FIG. 9 reveals that predictions made by using the methods of the present invention are both good, and that the expansion from P−4/P+3 to P−7/P+6 gives modestly improved predictions. In contrast, the pattern observed with the Scansite prediction includes many more peptides that are located at positions far from the optimal correlation.

FIG. 10 tabulates the results obtained. As shown in FIG. 10, the methods of the invention have approximately 90% specificity and sensitivity while the methods provided by Scansite have only 70% specificity and 45% sensitivity. Thus, the methods provided by the invention for predicting kinase specificity are better than this prior art approach for predicting PKC-delta specificity, even though the analysis was weighted in favor of the Cantley approach by using PKC-delta, which was exactly the kinase that Cantley used, and only a close relative of the kinase used in the methods of the invention (PKC-theta).

Identification of Peptides Efficiently Phosphorylated by PKC

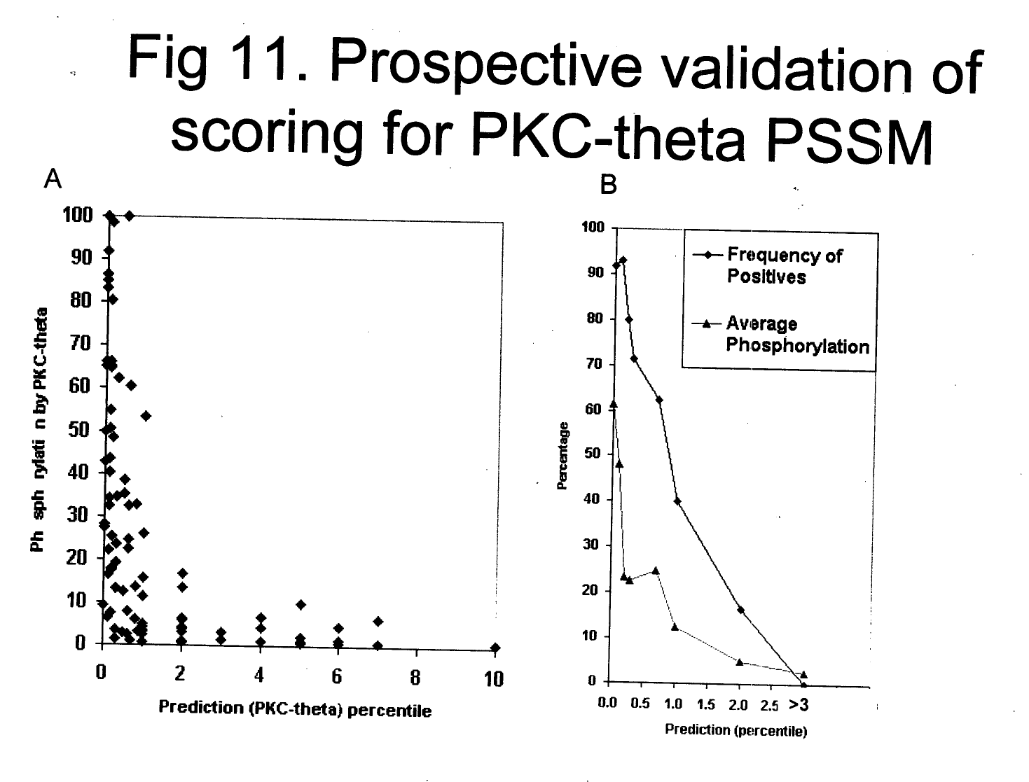

A second strategy for validation of the PSSM derived from the methods described herein is to identify sequences represented in the human proteome that have low percentiles derived from the PSSM, to synthesize peptides that have those sequences, and test the efficiency of phosphorylation of those peptides by the kinase of interest. FIG. 11 shows the results for such an analysis for 96 individual peptides. The results are shown for individual peptides (FIG. 11, panel A) or for groups of peptides aggregated by percentile prediction (FIG. 11, panel B). As with the testing described above with prospectively chosen peptides, the percentile scores are highly predictive of phosphorylation by the relevant kinase.

The process of prediction and testing resulted in identification of many peptides predicted to be substrates for PKC-theta and demonstrated to be substrates for PKC-theta (Table 3). A number of the sequences surrounding the most likely phosphorylation site have quite incomplete matches to the prototypic PKC substrate pattern [RK][RK]x[ST][hydrophobic][RK][RK]. Most of these peptides/sites have not previously been reported to be substrates for PKC in vivo or in vitro.

TABLE 3

Identification of in vitro substrates of
PKC-theta with further method validation

| | | | | Measured in vitro | Prediction |
|---|---|---|---|---|---|
| | SEQ | | | phosphorylation | from PKC- |
| Sequence | ID NO | LocusLinkID | Name | by PKC-theta | theta |
| -AMSRSA-S-KRRSR- | 168 | 7074 | TIAM1 | 100 | 0.5 |
| RTRSRRL-T-FRK--- | 169 | 1901 | S1P1 receptor | 100 | 0.0 |
| --VKLRR-S-KKRTKR | 170 | 1794 | DOCK2 | 98 | 0.1 |
| --RRGRRSTKKRRR | 171 | 55672 | FLJ20719 | 92 | 0.0 |
| --VRRRRSQRISQR | 172 | 25836 | IDN3 | 86 | 0.0 |
| RSGRRRGSQKS--- | 173 | 202 | absent in | 85 | 0.0 |
| | | | melanoma 1 | | |
| KKERRRNSINRN-- | 174 | 4542 | myosin IF | 83 | 0.0 |
| -KKRRTKSSRRGV- | 175 | 1612 | DAP-kinase 1 | 80 | 0.1 |
| ---RRERSRSRRKQ | 176 | 2305 | forkhead | 66 | 0.1 |
| | | | (Drosophila)- | | |
| | | | like 16 | | |
| -RRRRRRSRTFSR- | 177 | 1196 | CLK2 | 66 | 0.0 |
| ---RRRRSRTFSRS | 178 | 1196 | CLK2 | 65 | 0.0 |
| -KRHYRKSVRSRS- | 179 | 65125 | WNK1 | 65 | 0.1 |
| -FLRRSSSRRNRS- | 180 | 9595 | PSCDBP | 65 | 0.1 |
| TGERKRKSVRG--- | 181 | 6194 | ribosomal | 62 | 0.3 |
| | | | protein S6 | | |
| -TKKKRGSYRGGS- | 182 | 9221 | nucleolar | 61 | 0.6 |
| | | | phosphoprotein | | |
| | | | p130 | | |
| -ARRSKRSRRRET- | 183 | 23031 | MAST3 | 55 | 0.1 |
| ----FRASSRSTTK | 184 | 4863 | NPAT | 54 | 1.0 |
| KKFKRRLSLTLR-- | 185 | 5128 | PCTK2 | 51 | 0.1 |
| -DFRRRRSFRRIA- | 186 | 5734 | prostaglandin | 50 | 0.0 |
| | | | E receptor 4 | | |
| --LRRKSSTRHIHA | 187 | 672 | BRCA1 | 48 | 0.2 |
| -ERGRRGSKKGSI- | 188 | 695 | BTK | 44 | 0.1 |
| GRRRRSRSKVK--- | 189 | 8899 | serine/ | 43 | 0.0 |
| | | | threonine- | | |
| | | | protein | | |
| | | | kinase PRP4 | | |
| --RRRRHTMDKDSR | 190 | 65125 | WNK1 | 40 | 0.1 |
| ---HKRNSVRLVIR | 191 | 409 | beta-arrestin2 | 38 | 0.5 |
| -GNRKGKSKKWRQ- | 192 | 2870 | GRK6 | 35 | 0.5 |
| --PLRKSSLKKGGR | 193 | 393 | ARHGAP4 | 35 | 0.3 |
| -KRRKRKSLQRHK- | 194 | 1455 | casein kinase | 34 | 0.1 |
| | | | I gamma 2 | | |
| PGSSHRKTKK---- | 195 | 695 | BTK | 33 | 0.8 |
| -RWKRRRSYSREH- | 196 | 1198 | CLK3 | 32 | 0.1 |
| -ILRPSKSVKLRS- | 197 | 26191 | Lyp | 32 | 0.6 |
| --RRRRPTKSKGSK | 198 | 65125 | WNK1 | 28 | 0.0 |
| -RGRRSRSRLRRR- | 199 | 8899 | serine/ | 27 | 0.0 |
| | | | threonine- | | |
| | | | protein | | |
| | | | kinase PRP4 | | |
| EQQRRALSFRQ--- | 200 | 5778 | HePTP | 26 | 1.0 |
| -TQDRRKSLFKKI- | 201 | 23031 | MAST3 | 25 | 0.2 |
| -VMKRKFSLRAAE- | 202 | 6840 | supervillin | 24 | 0.6 |
| -VRRSKKSKKKES- | 203 | 23227 | MAST4 | 24 | 0.3 |
| RFSRRSSSWRIL-- | 204 | 4033 | LRMP | 22 | 0.6 |
| -EGRRSRSRRYSG- | 205 | 1105 | CHD1 | 22 | 0.1 |
| KSSRNSTSVKKK-- | 206 | 9934 | GPR105 | 19 | 0.3 |
| -SFRGHITRKKLK- | 207 | 2596 | gap-43 | 18 | 0.2 |
| -VSRPRKSRKRVD- | 208 | 25836 | IDN3 | 17 | 0.2 |
| DKEKSKGSLKRK-- | 209 | 5777 | SHP-1 | 17 | 2.0 |
| -PLRRRESMHVEQ- | 210 | 6650 | SOLH | 16 | 0.1 |
| RSRSYSRSRSR--- | 211 | 4820 | NKTR | 16 | 1.0 |
| --VSRGSSLKILSK | 212 | 7852 | CXCR4 | 13 | 2.0 |
| -RHSRSRSRHRLS- | 213 | 8621 | CDC2L5 | 13 | 0.8 |
| -SRRRSPSYSRHS- | 214 | 8621 | CDC2L5 | 13 | 0.3 |
| -TKKRSKSRSKER- | 215 | 8899 | serine/ | 12 | 0.5 |
| | | | threonine- | | |
| | | | protein | | |
| | | | kinase PRP4 | | |
| --SCRTSSRKRAGK | 216 | 8915 | BCL10 | 11 | 1.0 |

Considerations in Design of Test Sets of Peptides

Design of each test set of peptides involves important decisions regarding: the choice of phosphorylatable residue, the choice of anchor positions, the identity of residues at the anchor positions, the choice of the query positions, the identity of residues for the query positions and choice of positions and residue types for the degenerate positions. These considerations are discussed in more detail below.

In most embodiments, one position is a residue that can be phosphorylated (a phosphorylatable amino acid position), such as serine (S), threonine (T) or tyrosine (Y). As described above such a phosphorylatable position is referred to as “P0.” The choice between S, T and Y is based on the known or inferred phosphorylation preference of the kinase(s) whose specificity is to be assessed. For example, protein kinase C (PKC) phosphorylates a serine (S) more often than threonine (T). However, data obtained by the inventors indicates that Rho-kinase generally phosphorylates a threonine (T) and it has been previously determined that Lck generally phosphorylates a tyrosine (Y). Hence, one of skill in the art can use available information to assign the identity of the phosphorylatable amino acid. Alternatively, procedures like those provided herein or other available procedures can be used to determine which residues are preferentially phosphorylated by a kinase of unknown specificity.

Selecting the Number and Identity of Anchor Positions.

Anchor positions in the peptides used in the present methods can be at any position within the sequence of a test peptide pool. In particular, anchor positions do not need to be contiguous (i.e. next) to each other in the present methods. Anchor positions need not be adjacent to the query amino acid position. Anchor positions also do not need to be adjacent to the phosphorylatable residue. For example, many of the test sets in the superset of peptides used for PKC analysis had anchor residues in the pattern Rxx-S-F (see FIG. 2) where the anchor residue arginine (R) was adjacent neither to the phosphorylatable residue serine (S) nor to the other anchor residue phenylalanine (F).

The number of anchor positions selected for a set of peptides can influence the amount of information obtained about the substrate. In general, if too many residues are anchored then the test set will be relatively insensitive to changes in the query residues. However, if too few residues are anchored then the average amount of phosphorylation in the set will be too low. Low levels of phosphorylation can lead to error-prone readings. For example, when there is a low level of phosphorylation, decreases in phosphorylation caused by disfavored query residues will generally be small and unreliable.

In most embodiments, one or two positions are assigned to be anchor positions. However, a larger number of anchor residues can be useful in some embodiments, particularly those designed for particular conditions. As illustrated herein some embodiments have two anchor positions. For example, two anchor residues were used for six of the eight test sets in a superset design for PKC analysis, i.e. R??-S-F?? (FIG. 2). As show herein, use of this superset provides a good characterization of the specificity of PKCs.

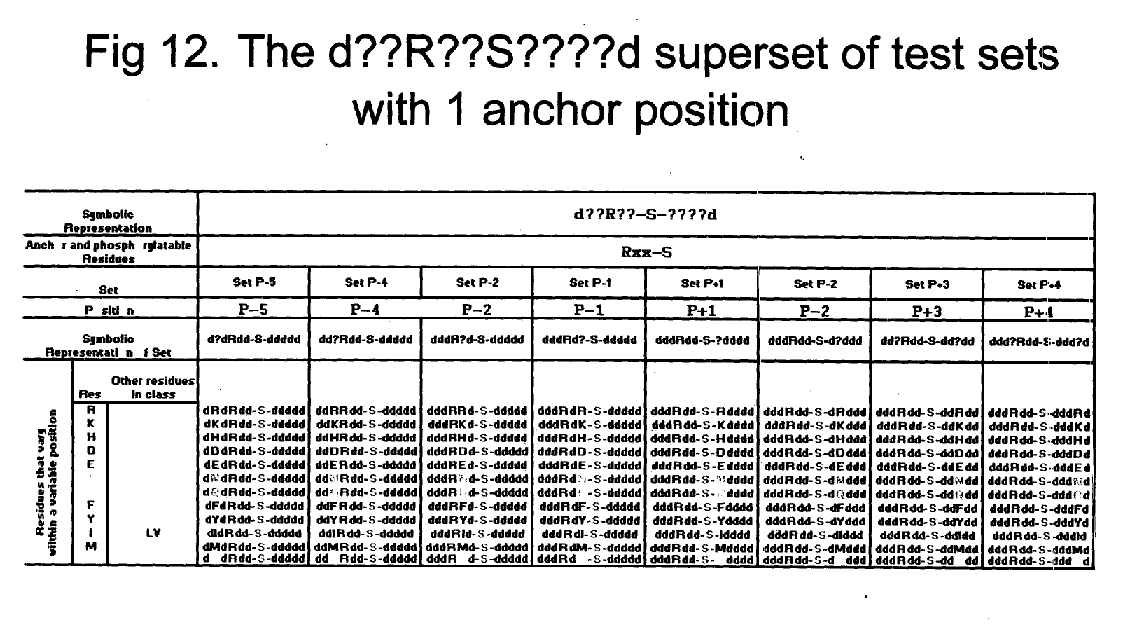

Supersets with one anchor position are also very useful. The utility of such a superset with one anchor position is illustrated by a superset consisting of 8 test sets with the symbolic representation d??R??S????d (FIG. 12). This d??R??S????d superset is an especially useful superset for initial characterization of kinases that may be basophilic, because many basophilic kinases have a strong preference for ‘R’ at the P−3 position.

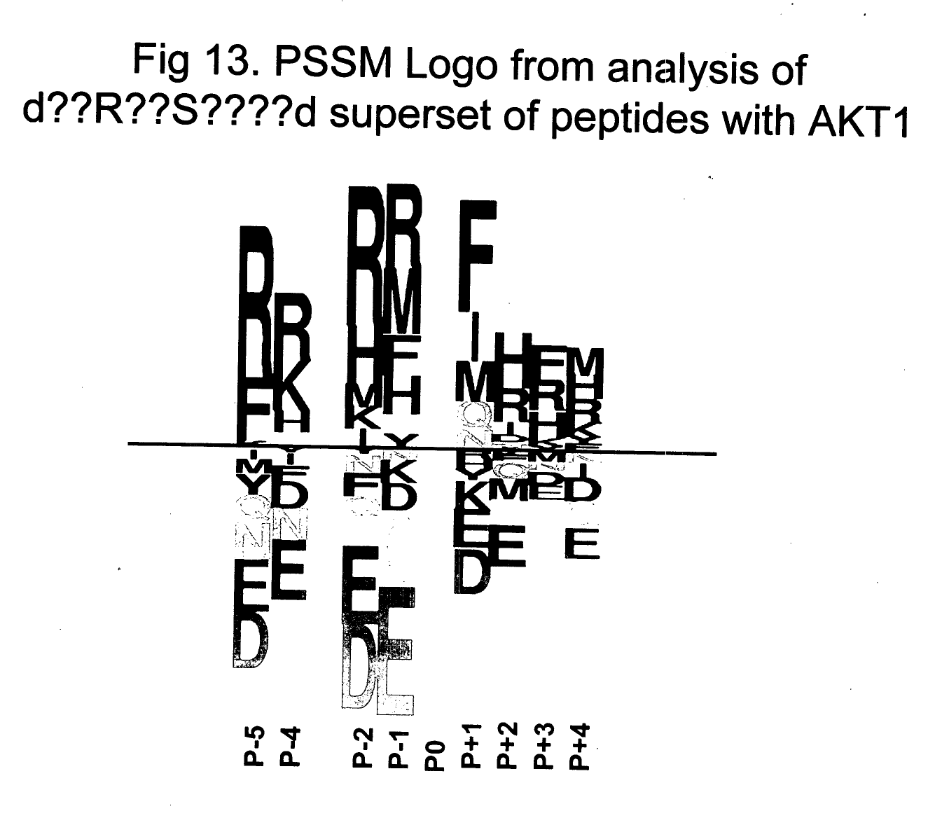

FIG. 13 shows a PSSM Logo for analysis of the kinase AKT1 with this superset, which provides a good overview of the preferences of AKT1 at most positions between P−5 and P+4. Because there is only one anchor residue, the counts per minute for this superset after phosphorylation are typically lower than with two suitable anchor positions. However, this superset can still provide an adequate “dynamic range” showing favored and disfavored residues (FIG. 13). Data from this analysis provides an approximation of the specificity of AKT1. If more precise understanding is required, then a suitable second anchor position can be chosen from the results of this d??R??S????d set, and an additional superset(s) of test peptides can be synthesized with two anchor positions. One of skill in the art can envision other one-anchor sets that would be especially useful such as d?????SP???d for proline-directed kinases, d?????SQ????d for ‘SQ’ directed kinases, and d?????SR???d for ‘SR’ directed kinases.

According to the invention, several principles for choosing a second anchor position from the results of a one anchor set such as d??R??S????d. In general, the second anchor is an amino acid that is strongly preferred by the kinase of interest. In the case of AKT1, illustrated by FIG. 13, there are multiple such residues, for example, R at P−5, R at P−2, and F at P+1. In choosing between those, a secondary consideration is minimizing the number of other preferred residues at that position. Hence, a second anchor amino acid is selected as the most preferred of only a few preferred residues at that position. Based on that criterion, a particularly good choice would be R at P−5. If one of skill in the art wishes to obtain more detailed information on which anchor residues to select, multiple second anchors can be chosen and supersets synthesized to test each anchor position.

It is also important to note that a superset based on no anchors, such as d????S????d or d????Y????d can also be useful. Information derived by analysis with such a set could be particularly useful for choice of a second anchor (distinct from R at P−3) on which to build a superset conceptually similar to the d??R??S????d superset.

If sufficient prior knowledge is available, the anchor residues for test sets can be chosen based on that prior knowledge. The choice of anchor positions and anchor residue identities for the RxxSF PKC-theta supersets (FIG. 2 and FIG. 6) were based on prior knowledge of the inventor on PKC specificity in which the dominant residues that determine PKC specificity were believed to include arginine at P−3, arginine at P−2, phenylalanine at P+1, arginine at P+2 and arginine at P+3. Therefore, some or all of such previously identified residues and/or positions can be chosen for the anchor positions of a particular test set or superset of peptides.

Choice of the Query Positions and the Amino Acid Residues at the Query Position.

In most embodiments each test set has only one query position. This assures that the difference between peptides in the test set can be clearly attributed to change in a single amino acid at a standardized position.

Of importance in the current method is the fact that the query position does NOT need to be adjacent to either an anchor position or to a phosphorylatable position. This contrasts with pervasive use in the prior art of query positions adjacent to anchor positions (and phosphorylatable positions) in methods using “systematic amino acid variation on template substrate” (SAaVoTS). Particularly notable is that the extensive work of Tegge and colleagues on finding optimal peptides/inhibitors was based on query residues adjacent to fixed residues (for example Dostmann W R et al. 1999. Pharmacol Ther 82:373-387; Tegge W et al. 1995. Biochemistry 34:10569-10577; Tegge W J et al. 1998. Methods Mol Biol 87:99-106). Thus, the current method incorporates new flexibility relative to the prior art of “systematic amino acid variation on template substrate” by placing a query position at any position relative to the anchor and phosphorylatable positions.

Any amino acid can be selected for placement at the query position. While in some embodiments all available amino acids are systematically placed and tested in the query position, in other embodiments only a subset of natural amino acids are selected for placement in the query position. Hence, in some embodiments, the test set of peptides would include one peptide for each natural amino acid. In other embodiments, cysteine is eliminated and only nineteen alternative amino acid residues are used.

In other embodiments, economy is achieved by assuming that amino acids can be subdivided into classes that are most similar in their functional properties. For example, using this strategy, a “reduced set” of only about thirteen amino acid residues are alternatively placed in the query position, as illustrated by FIG. 2 and FIG. 6. For example, one of skill in the art may choose to eliminate glutamic acid (E) by virtue of its similarity to aspartic acid (D); isoleucine (I), methionine (M) and valine (V) can be eliminated by virtue of their similarity to leucine (L) and tyrosine (Y) can be eliminated by virtue of similarity to phenylalanine (F) (see further details in Example 2).

Choosing Residues and Conditions for Degenerate Positions

The degenerate amino acid position in the peptide pools can be created such that any one of the twenty amino acids can occupy that position. However, this strategy can be altered by one of skill in the art to suit the needs of a particular test or situation. For example, one of skill in the art may elect not to use cysteine because can give rise to disulfide bonds and dimer formation.

In other embodiments, residues that may be phosphorylated (e.g. S, T, and Y) can be excluded from the degenerate positions. However, serine, threonine and tyrosine residues may also be included because they can have a role in determining substrate specificity and because an experimental design minimizes noise when such residues are used in degenerate position. For example, in the methods of the invention noise from degenerate position serine, threonine or tyrosine residues is minimized because of the abundance of the selected serine, threonine, or tyrosine residue at the P0 position relative to the rarity of these amino acids in degenerate positions. Moreover, phosphorylation at the P0 position is selectively enhanced by the anchor residues that guide the kinase to phosphorylate the appropriate residue. Hence, the types and positions of degenerate residues can be varied as needed.

Two approaches can be used for inserting a degenerate set of amino acids into selected positions of a peptide. In one embodiment, a mixture of selected amino acid residues is added by a specific coupling step to create a degenerate position. However, different amino acid residues have different coupling efficiencies and therefore, if equal amounts of each amino acid are used, each amino acid residue may not be equivalently represented at the degenerate position. The different coupling efficiencies of different amino acids can be compensated for by using a “weighted” mixture of amino acids at a coupling step, wherein amino acids with lower coupling efficiencies are present in greater abundance than amino acids with higher coupling efficiencies. Conditions of the coupling can also be varied to facilitate achievement of a desired mix in the synthesized peptide. For example relatively low molar ratios minimize skewing by different coupling efficiencies; also, repetitive additions of low molar ratios can augment efficiency while minimizing skewing.

In an alternative embodiment, the resin upon which the peptides are synthesized is divided into equivalent portions and then each portion is subjected to a separate coupling reaction that employs a distinct type of amino acid. After this coupling reaction, the resin aliquots are recombined and the procedure is repeated for each degenerate position. This approach results in approximately equivalent representation of each different amino acid residue at the degenerate position.

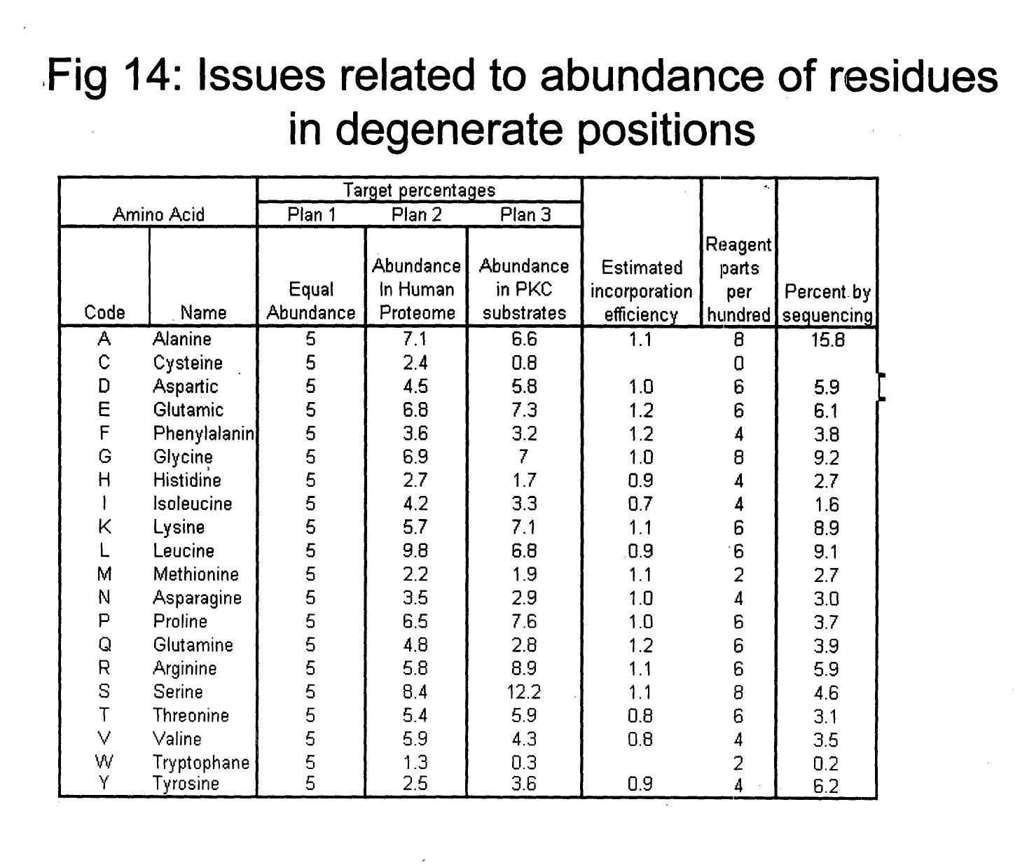

The abundance of residues at the degenerate positions in the peptides can be controlled by a variety of different strategies (see FIG. 14). One procedure for controlling the abundance of residues at the degenerate position is shown as plan 1 in FIG. 14, where an equal abundance of each amino acid residue is selected for each position. However, in many embodiments the abundance of amino acids is based on prior knowledge of the abundance of residues in human proteins or relevant regions thereof. One such embodiment utilized the average abundance of various amino acids in the human proteome. The abundance of amino acids in human proteins was determined by reference to sequences tabulated by the National Center for Biotechnology Information (Plan 2, FIG. 14).

In another embodiment, the abundance of various amino acids at a degenerate position correlates with the abundance of that amino acid in known kinase substrates (Plan 3, FIG. 14). Plan 3 of FIG. 14 takes into account the physiological relevance of various residues and resembles the residue abundance found in physiologic substrates for the kinase(s). To this end, the inventor has accumulated a list of known or suspected substrate sites for PKC and has determined the residue frequency in the regions surrounding those sites (Plan 3, FIG. 14). The intent was to create a method that screens the most relevant peptide sequences for targeted biological processes.

Hence, in some embodiments a degenerate mixture of residues is used that is like the types of amino acid residues thought to be most relevant to a particular kinase. Implementing this improvement by deviating from equal abundance is not a problem in the present method but could be a problem in prior art approaches (e.g. U.S. Pat. No. 6,004,757 to Cantley) because prior art approaches depend on detection of substrate residue by sequence analysis of the phosphorylated product and a low abundance of a particular residue in the degenerate peptide pool being phosphorylated would decrease the reliability of detecting such a difference.

Additional Residues Beyond the Core Peptide

The peptide pools in a test set or in a superset can include additional residues at either the N-terminus or C-terminus (or both). Such additional amino acid residues may provide additional attachment points or other functions useful to one of skill in the art. For example, in the ninety peptide test set having the formula Rxx-S-F, each peptide included a three residue N-terminal linker of biotinylated lysine, dansylated lysine and glycine. The biotin moiety provided an efficient mechanism for capture of the peptide before, during or after an assay. The dansyl moiety also provided a convenient means to quantify the amount of each peptide by measuring light absorption at 335 nm. The glycine provided flexibility in connecting the linker to the remainder of the peptide. Hence, such linkers can be used in the methods, articles and kits of the invention.

Examples of Other Variations in Tests Sets of Peptides

The number of peptide pools in a test set can vary. In some embodiments, the number of peptide pools in the test set is equivalent to the number of amino acids tested at the query position. Hence, for example, if all twenty naturally-occurring amino acids are tested in the test set, the number of peptide pools would be twenty. However, in many embodiments, fewer than twenty amino acids are tested because one of skill in the art may have information indicating that certain amino acids need not be tested. Moreover, many amino acid analogs are available to one of skill in the art and in some instances the skilled artisan may choose to test such an amino acid analog at the query position. In such instances, amino acid analogs may be used in the test sets of the invention and the number of peptide pools can be greater than twenty. Also, under special circumstances it is useful to use a mixture of amino acids, such as (R+K) or (D+E) instead of a single amino acid at a query position. Similarly, special circumstances may dictate use of a limited mix of amino acids at the phosphorylatable position (such as S+T), or at an anchor position (such as I+L+M+V). Note that FIG. 2 illustrates that the same degenerate peptide can be used in three different sets: for example, the peptide symbolized by ‘ddddRdd-S-Fdd’ (shaded) was an element of the P−3 set, the P−0 set, and the P+1 set.

The number of test sets in a superset or collection of peptide pools can also vary. In general a superset has at least two test sets of peptide pools. Typically the number of test sets corresponds to the number of positions around the phosphorylation site that are being tested, which is usually in the range of from about five to about twenty positions (or test sets). Moreover, a given test set can be used as part of different supersets. Also, practical considerations such as number of wells in a standardized plate (e.g. 96 or 384) often contribute to the choices made regarding number peptide pools in a test set, and number of test sets in a superset. Moreover, different test sets can be used as part of different supersets.

The length of a peptide in a peptide pool can also vary. For example, although the amino acid sequences described in this application are often about five to about fifteen amino acids in length, a peptide that is shorter than five amino acids may be used in some embodiments. For example, a peptide as short as about three amino acids in length may be used as a substrate. The upper size of the peptides used in the test sets and supersets is not critical and can vary as desired by one of skill in the art. However, peptides that are chemically synthesized become more expensive as their length increases. Hence, one of skill in the art may choose to limit the size of the peptides employed to about 100 or fewer amino acids, or about 50 or fewer amino acids, or about 30 or fewer amino acids, or about 25 or fewer amino acids.

In some embodiments the peptide pools used in the test sets and supersets of the invention are soluble pools of peptides. The term “soluble peptide pools” is intended to mean a population of peptides that are not attached to a solid support at the time they are subjected to phosphorylation.

In alternative embodiments, the peptides used in the test sets and supersets of the invention can be attached to a solid support such as a bead, a well of a microtiter dish, a membrane or a plastic pin. For general descriptions of the construction of solid-support bound peptide libraries see for example Geysen, H. M., et al. (1986) Mol. Immunol. 23:709-715; Lam, K. S., et al. (1991) Nature 354:82-84; and Pinilla, C., et al. (1992) BioTechniques 13:901-905. For this type of library, the peptides can be synthesized while attached to a solid support such as a bead, and degenerate positions are created by splitting the population of beads, coupling different amino acids to different subpopulations and recombining the beads. The final product is a population of beads each carrying many copies of a single unique peptide. This approach has been termed “one bead/one peptide”.

The choice of a soluble versus immobilized format should not be based solely on convenience of the assay; some studies conducted by the inventors suggest that significant differences in specificity are observed with the same peptides assayed in solution versus assays performed on immobilized peptides. Therefore, the distinction between soluble and immobilized may be of considerable importance. The use of soluble peptide pools as the preferred embodiment of this invention distinguishes the invention from many prior methods performed with immobilized peptides. Also, those of skill in the art should carefully assess all the implications of these alternative formats when choosing the design of test sets of peptides for particular applications.

The peptides utilized in the test sets and supersets of the invention can be prepared by any method available to one of skill in the art. For example, the peptides can be constructed by in vitro chemical synthesis, for example using an automated peptide synthesizer. As described herein the peptides can be soluble peptide pools or the peptides can be attached to a solid support such as a bead, membrane, microtiter well, tube or other convenient solid support.

Standard techniques for in vitro chemical synthesis of peptides are known in the art. For example, peptides can be synthesized by (benzotriazolyloxy)tris (dimethylamino)-phosphonium hexafluorophosophate (BOP)/1-hydroxybenzotriazole coupling protocols. Automated peptide synthesizers are commercially available (e.g., Milligen/Biosearch 9600). For general descriptions of the construction of soluble synthetic peptide libraries see for example Houghten, R. A., et al., (1991) Nature 354:84-86 and Houghten, R. A., et al., (1992) BioTechniques 13:412-421.

Binding Entities that Bind to Substrates of Kinases

The invention also contemplates binding entities that can bind to peptides or proteins that may be phosphorylated by a kinases. In some embodiments, the binding entities bind to the non-phosphorylated substrate; in other embodiments the binding entities bind to phosphorylated substrates. Such binding entities can be used in vitro or in vivo for detecting phosphorylated or non-phosphorylated peptide or protein or modulating the function of a phosphorylated or non-phosphorylated protein. As used herein, a binding entity is any small molecule, peptide, or polypeptide that can bind to a peptidyl substrate site of kinase. In some embodiments, the binding entities are antibodies.

Hence, binding entities can bind to a phosphorylated peptidyl substrate sequence but exhibit significantly less or substantially no binding to the corresponding non-phosphorylated peptidyl substrate sequence. Binding entities of the invention can also bind to a non-phosphorylated peptidyl substrate sequence but exhibit significantly less or substantially no binding to the corresponding phosphorylated peptidyl substrate sequence.

For example, binding entities and antibodies contemplated by the invention may bind to a peptide having one or a few of SEQ ID NO: 76, 79, 81, 82, 87, 89-94, 97, 98, 100, 102, 104, 105, 108, 110, 112, 113, 115, 117, 121, 124, 125, 127-134, 136, 138, 139, 143-145, 148-153, 156, 160, 163-180, 182-194, 196-206, 208-211, 213-216. In another embodiment, binding entities and antibodies of the invention bind to one of peptide SEQ ID NO: 76, 79, 81, 82, 87, 89-94, 97, 98, 100, 102, 104, 105, 108, 110, 112, 113, 115, 117, 121, 124, 125, 127-134, 136, 138, 139, 143-145, 148-153, 156, 160, 163-180, 182-194, 196-206, 208-211, 213-216, but not any other of the peptides. In further embodiments of the invention, binding entities and antibodies of the invention bind to a phosphorylated peptide having one of SEQ ID NO: 76, 79, 81, 82, 87, 89-94, 97, 98, 100, 102, 104, 105, 108, 110, 112, 113, 115, 117, 121, 124, 125, 127-134, 136, 138, 139, 143-145, 148-153, 156, 160, 163-180, 182-194, 196-206, 208-211, 213-216, but exhibit significantly less or substantially no binding to the corresponding non-phosphorylated peptidyl substrate sequence. Other examples of phosphorylated peptides to which the binding entities and antibodies of the invention can bind include phosphorylated peptides having SEQ ID NO:298-347, 349-473.

In still further embodiments of the invention, binding entities and antibodies of the invention bind to a non-phosphorylated peptide having one of SEQ ID NO: 76, 79, 81, 82, 87, 89-94, 97, 98, 100, 102, 104, 105, 108, 110, 112, 113, 115, 117, 121, 124, 125, 127-134, 136, 138, 139, 143-145, 148-153, 156, 160, 163-180, 182-194, 196-206, 208-211, 213-216, but exhibit significantly less or substantially no binding to the corresponding phosphorylated peptidyl substrate sequence.

The invention provides antibodies and binding entities made by available procedures that can bind a peptide or phosphorylated peptide of the invention. The binding domains of such antibodies, for example, the CDR regions of these antibodies, can also be transferred into or utilized with any convenient binding entity backbone.

Antibody molecules belong to a family of plasma proteins called immunoglobulins, whose basic building block, the immunoglobulin fold or domain, is used in various forms in many molecules of the immune system and other biological recognition systems. A standard antibody is a tetrameric structure consisting of two identical immunoglobulin heavy chains and two identical light chains and has a molecular weight of about 150,000 daltons.

The heavy and light chains of an antibody consist of different domains. Each light chain has one variable domain (VL) and one constant domain (CL), while each heavy chain has one variable domain (VH) and three or four constant domains (CH). See, e.g., Alzari, P. N., Lascombe, M.-B. & Poljak, R. J. (1988) Three-dimensional structure of antibodies. Annu. Rev. Immunol. 6, 555-580. Each domain, consisting of about 110 amino acid residues, is folded into a characteristic β-sandwich structure formed from two β-sheets packed against each other, the immunoglobulin fold. The VH and VL domains each have three complementarity determining regions (CDR1-3) that are loops, or turns, connecting β-strands at one end of the domains. The variable regions of both the light and heavy chains generally contribute to antigen specificity, although the contribution of the individual chains to specificity is not always equal. Antibody molecules have evolved to bind to a large number of molecules by using six randomized loops (CDRs).

Immunoglobulins can be assigned to different classes depending on the amino acid sequences of the constant domain of their heavy chains. There are at least five (5) major classes of immunoglobulins: IgA, IgD, IgE, IgG and IgM. Several of these may be further divided into subclasses (isotypes), for example, IgG-1, IgG-2, IgG-3 and IgG-4; IgA-1 and IgA-2. The heavy chain constant domains that correspond to the IgA, IgD, IgE, IgG and IgM classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of variable domains differ extensively in sequence from one antibody to the next. The variable domains are for binding and determine the specificity of each particular antibody for its particular antigen. However, the variability is not evenly distributed through the variable domains of antibodies. Instead, the variability is concentrated in three segments called complementarity determining regions (CDRs), also known as hypervariable regions in both the light chain and the heavy chain variable domains.

The more highly conserved portions of variable domains are called framework (FR) regions. The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from another chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific peptide sequence described herein or a derivative thereof.

Moreover, the binding regions, or CDR, of antibodies can be placed within the backbone of any convenient binding entity polypeptide. In preferred embodiments, in the context of methods described herein, an antibody, binding entity or fragment thereof is used that is immunospecific for any of the peptides described herein, as well as the derivatives thereof, including the phosphorylated derivatives thereof.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called Fab fragments, each with a single antigen binding site, and a residual Fe fragment. Fab fragments thus have an intact light chain and a portion of one heavy chain. Pepsin treatment yields an F(ab′)₂ fragment that has two antigen binding fragments that are capable of cross-linking antigen, and a residual fragment that is termed a pFc′ fragment. Fab′ fragments are obtained after reduction of a pepsin digested antibody, and consist of an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. As used herein, “functional fragment” with respect to antibodies, refers to Fv, F(ab) and F(ab′)₂ fragments.

Additional fragments can include diabodies, linear antibodies, single-chain antibody molecules, and multispecific antibodies formed from antibody fragments. Single chain antibodies are genetically engineered molecules containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, where the fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

Antibody fragments contemplated by the invention are therefore not full-length antibodies. However, such antibody fragments can have similar or improved immunological properties relative to a full-length antibody. Such antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more.

In general, an antibody fragment of the invention can have any upper size limit so long as it is has similar or improved immunological properties relative to an antibody that binds with specificity to a peptide or phosphorylated peptide described herein. For example, smaller binding entities and light chain antibody fragments can have less than about 200 amino acids, less than about 175 amino acids, less than about 150 amino acids, or less than about 120 amino acids if the antibody fragment is related to a light chain antibody subunit. Moreover, larger binding entities and heavy chain antibody fragments can have less than about 425 amino acids, less than about 400 amino acids, less than about 375 amino acids, less than about 350 amino acids, less than about 325 amino acids or less than about 300 amino acids if the antibody fragment is related to a heavy chain antibody subunit.

Antibodies directed against disease markers can be made by any available procedure. Methods for the preparation of polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

Monoclonal antibodies can also be employed in the invention. The term “monoclonal antibody” as used herein refers to an antibody obtained from a population of substantially homogeneous antibodies. In other words, the individual antibodies comprising the population are identical except for occasional naturally occurring mutations in some antibodies that may be present in minor amounts. Monoclonal antibodies are highly specific, being directed against a single antigenic site. Furthermore, in contrast to polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass. Fragments of such antibodies can also be used, so long as they exhibit the desired biological activity. See U.S. Pat. No. 4,816,567; Morrison et al. Proc. Natl. Acad. Sci. 81, 6851-55 (1984). The monoclonal antibodies herein also specifically include those made from different animal species, including mouse, rat, human and rabbit.

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of antibodies are available to those skilled in the art. For example, the monoclonal antibodies to be used in accordance with the present invention may be made by the hybridoma method as described above or may be made by recombinant methods, e.g., as described in U.S. Pat. No. 4,816,567. Monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression of nucleic acids encoding the antibody fragment in a suitable host. Antibody fragments can be obtained by pepsin or papain digestion of whole antibodies conventional methods. For example, antibody fragments can be produced by enzymatic cleavage of antibodies with pepsin to provide a 5S fragment described as F(ab′)₂. This fragment can be further cleaved using a thiol reducing agent, and optionally using a blocking group for the sulfhydryl groups resulting from cleavage of disulfide linkages, to produce 3.5S Fab′ monovalent fragments. Alternatively, enzymatic cleavage using pepsin produces two monovalent Fab′ fragments and an Fc fragment directly. These methods are described, for example, in U.S. Pat. No. 4,036,945 and No. 4,331,647, and references contained therein. These patents are hereby incorporated by reference in their entireties.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

While standardized procedures are available to generate antibodies, the size of antibodies, the multi-stranded structure of antibodies and the complexity of six binding loops present in antibodies constitute a hurdle to the improvement and the manufacture of large quantities of antibodies. Hence, the invention further contemplates using binding entities, which comprise polypeptides that can recognize and hind to kinase substrates provided herein.

A number of proteins can serve as protein scaffolds to which binding domains can be attached and thereby form a suitable binding entity. The binding domains bind or interact with the peptide sequences of the invention while the protein scaffold merely holds and stabilizes the binding domains so that they can bind. A number of protein scaffolds can be used. For example, phage capsid proteins can be used. See Review in Clackson & Wells, Trends Biotechnol. 12:173-184 (1994). Phage capsid proteins have been used as scaffolds for displaying random peptide sequences, including bovine pancreatic trypsin inhibitor (Roberts et al., PNAS 89:2429-2433 (1992)), human growth hormone (Lowman et al., Biochemistry 30:10832-10838 (1991)), Venturini et al., Protein Peptide Letters 1:70-75 (1994)), and the IgG binding domain of Streptococcus (O'Neil et al., Techniques in Protein Chemistry V (Crabb, L., ed.) pp. 517-524, Academic Press, San Diego (1994)). These scaffolds have displayed a single randomized loop or region that can be modified to include binding domains for kinase substrates.

Researchers have also used the small 74 amino acid a-amylase inhibitor Tendamistat as a presentation scaffold on the filamentous phage M13. McConnell, S. J., & Hoess, R. H., J. Mol. Biol. 250:460-470 (1995). Tendamistat is a β-sheet protein from Streptomyces tendae. It has a number of features that make it an attractive scaffold for binding entities, including its small size, stability, and the availability of high resolution NMR and X-ray structural data. The overall topology of Tendamistat is similar to that of an immunoglobulin domain, with two β-sheets connected by a series of loops. In contrast to immunoglobulin domains, the β-sheets of Tendamistat are held together with two rather than one disulfide bond, accounting for the considerable stability of the protein. The loops of Tendamistat can serve a similar function to the CDR loops found in immunoglobulins and can be easily randomized by in vitro mutagenesis. Tendamistat is derived from Streptomyces tendae and may be antigenic in humans. Hence, binding entities that employ Tendamistat are preferably employed in vitro.

Fibronectin type III domain has also been used as a protein scaffold to which binding entities can be attached. Fibronectin type III is part of a large subfamily (Fn3 family or s-type Ig family) of the immunoglobulin superfamily. Sequences, vectors and cloning procedures for using such a fibronectin type III domain as a protein scaffold for binding entities (e.g. CDR peptides) are provided, for example, in U.S. Patent Application Publication 20020019517. See also, Bork, P. & Doolittle, R. F. (1992) Proposed acquisition of an animal protein domain by bacteria. Proc. Natl. Acad. Sci. USA 89, 8990-8994; Jones, E. Y. (1993) The immunoglobulin superfamily Curr. Opinion Struct. Biol. 3, 846-852; Bork, P., Hom, L. & Sander, C. (1994) The immunoglobulin fold. Structural classification, sequence patterns and common core. J. Mol. Biol. 242, 309-320; Campbell, I. D. & Spitzfaden, C. (1994) Building proteins with fibronectin type III modules Structure 2, 233-337; Harpez, Y. & Chothia, C. (1994).

In the immune system, specific antibodies are selected and amplified from a large library (affinity maturation). The combinatorial techniques employed in immune cells can be mimicked by mutagenesis and generation of combinatorial libraries of binding entities. Variant binding entities, antibody fragments and antibodies therefore can also be generated through display-type technologies. Such display-type technologies include, for example, phage display, retroviral display, ribosomal display, and other techniques. Techniques available in the art can be used for generating libraries of binding entities, for screening those libraries and the selected binding entities can be subjected to additional maturation, such as affinity maturation. Wright and Harris, supra., Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743.

The invention therefore also provides methods of mutating antibodies, CDRs or binding domains to optimize their affinity, selectivity, binding strength and/or other desirable properties. A mutant binding domain refers to an amino acid sequence variant of a selected binding domain (e.g. a CDR). In general, one or more of the amino acid residues in the mutant binding domain is different from what is present in the reference binding domain. Such mutant antibodies necessarily have less than 100% sequence identity or similarity with the reference amino acid sequence. In general, mutant binding domains have at least 75% amino acid sequence identity or similarity with the amino acid sequence of the reference binding domain. Preferably, mutant binding domains have at least 80%, more preferably at least 85%, even more preferably at least 90%, and most preferably at least 95% amino acid sequence identity or similarity with the amino acid sequence of the reference binding domain.

For example, affinity maturation using phage display can be utilized as one method for generating mutant binding domains. Affinity maturation using phage display refers to a process described in Lowman et al., Biochemistry 30(45): 10832-10838 (1991), see also Hawkins et al., J. Mol. Biol. 254: 889-896 (1992). While not strictly limited to the following description, this process can be described briefly as involving mutation of several binding domains or antibody hypervariable regions at a number of different sites with the goal of generating all possible amino acid substitutions at each site. The binding domain mutants thus generated are displayed in a monovalent fashion from filamentous phage particles as fusion proteins. Fusions are generally made to the gene III product of M13. The phage expressing the various mutants can be cycled through several rounds of selection for the trait of interest, e.g. binding affinity or selectivity. The mutants of interest are isolated and sequenced. Such methods are described in more detail in U.S. Pat. No. 5,750,373, U.S. Pat. No. 6,290,957 and Cunningham, B. C. et al., EMBO J. 13(11), 2508-2515 (1994).

Therefore, in one embodiment, the invention provides methods of manipulating binding entity or antibody polypeptides or the nucleic acids encoding them to generate binding entities, antibodies and antibody fragments with improved binding properties that recognize kinase substrate sequences.

Such methods of mutating portions of an existing binding entity or antibody involve fusing a nucleic acid encoding a polypeptide that encodes a binding domain for a disease marker to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage, and selecting phage that bind to a kinase substrate.

Accordingly, the invention provides antibodies, antibody fragments, and binding entity polypeptides that can recognize and bind to a kinase substrate (e.g., a peptide sequence having any one of SEQ ID NO: 76, 79, 81, 82, 87, 89-94, 97, 98, 100, 102, 104, 105, 108, 110, 112, 113, 115, 117, 121, 124, 125, 127-134, 136, 138, 139, 143-145, 148-153, 156, 160, 163-180, 182-194, 196-206,208-211, 213-216. The invention further provides methods of manipulating those antibodies, antibody fragments, and binding entity polypeptides to optimize their binding properties or other desirable properties (e.g., stability, size, ease of use).

Kinases that can be Used in the Methods of the Invention

The methods of the invention can be used to identify the specificity of any type of wild type or mutant kinase from any prokaryotic or eukaryotic species. For example, the kinase can be a protein-serine/threonine specific kinase (in which case a library with a fixed non-degenerate serine or threonine is used), a protein-tyrosine specific kinase (in which case a library with a fixed non-degenerate tyrosine is used) or a dual-specificity kinase (in which case a library with either a fixed non-degenerate serine, threonine or tyrosine can be used). Examples of protein kinases that can be utilized in the methods of the invention can also be found in Hanks et al. (1988) Science 241:42-52 and Manning G et al. 2002. Science 298:1912-1934.

Protein-serine/threonine specific kinases that can be used in the methods of the invention include: 1) cyclic nucleotide-dependent kinases, such as cyclic-AMP-dependent protein kinases (e.g., protein kinase A) and cyclic-GMP-dependent protein kinases; 2) calcium-phospholipid-dependent kinases, such as protein kinase C; 3) calcium-calmodulin-dependent kinases, including CaMII, phosphorylase kinase (PhK), myosin light chain kinases (e.g., MLCK-K, MLCK-M), PSK-H1 and PSK-C3; 4) the SNF1 family of protein kinases (e.g., SNF 1, nim1, KIN1 and KIN2); 5) casein kinases (e.g., CKII); 6) the Raf-Mos proto-oncogene family of kinases, including Raf, A-Raf, PKS and Mos; and 7) the STE7 family of kinases (e.g. STE7 and PBS2). Additionally, the protein-serine/threonine specific kinase can be a kinase involved in cell cycle control. Many kinases involved in cell cycle control have been identified. Cell cycle control kinases include the cyclin dependent kinases, which are heterodimers of a cyclin and kinase (such as cyclin B/p33^{cdc2}, cyclin A/p33^{CDK2}, cyclin E/p33^{CDK2} and cyclin D1/p33^{CDK4}). Other cell cycle control kinases include Wee1 kinase, Nim1/Cdr1 kinase, Wis1 kinase and NIMA kinase.

Protein-tyrosine specific kinases that can be used in the methods of the invention include: 1) members of the src family of kinases, including pp60^{c-src}, pp60^{v-src}, Yes, Fgr, FYN, LYN, LCK, HCK, Dsrc64 and Dsrc28; 2) members of the Abl family of kinases, including Abl, ARG, Dash, Nabl and Fes/Fps; 3) members of the epidermal growth factor receptor (EGFR) family of kinases, including EGFR, v-Erb-B, NEU and DER; 4) members of the insulin receptor (INS.R) family of growth factors, including INS.R, IGF1R, DILR, Ros, 7less, TRK and MET; 5) members of the platelet-derived growth factor receptor (PDGFR) family of kinases, including PDGFR, CSF1R, Kit and RET.

Other protein kinases which can be used in the method of the invention include syk, ZAP70, Focal Adhesion Kinase, erk1, erk2, erk3, MEK, CSK, BTK, ITK, TEC, TEC-2, JAK-1, JAK-2, LET23, c-fms, S6 kinases (including p70^{S6} and RSKs), TGF-β/activin receptor family kinases and Clk.

Kits

The invention is further directed to a kit having a test set or an array of peptide pools for identifying kinase substrate specificities. The peptides used in the test sets and arrays can be soluble peptides or peptides attached to a solid support. Instructions for using the array can also be included in the kit.

As described above, a test set contains peptide pools, wherein every peptide in each of the peptide pools has an amino acid that can be phosphorylated by a kinase, a query amino acid, at least one anchor amino acid, and at least one degenerate amino acid. The amino acid that can be phosphorylated by a kinase is at a defined phosphorylation position and every peptide of every peptide pool within a test set of peptide pools has an identical amino acid that can be phosphorylated by a kinase in that phosphorylation position. The query amino acid is at a defined query position within a test set but the query amino acid's identity at that defined query position is systematically varied from one peptide pool to the next peptide pool within a test set of peptide pools. Each anchor amino acid is at a defined anchor position within a test set and an identical anchor amino acid is present at that defined position in every peptide of every peptide pool in the test set, but each test set of the series of test sets can have different anchor amino acids. The at least one degenerate amino acid is an unknown amino acid selected from a degenerate mixture of amino acids.

The methods and kits of the invention can be used to determine an amino acid sequence motif for the phosphorylation site of any kinase. The preferred embodiment of such kits includes software to facilitate calculation of results, determination of derived parameters such as residue preference and scores for a position specific scoring matrix, and display of results in informative formats such as the PSSM Logo. The kits of the invention can also include any item, reagent or solution useful for performing the methods of the invention. Such items can include microtiter plates, arrays of peptide pools where the peptides are attached to a solid support, tubes for diluting reagents, and the like. Reagents useful for performing the methods of the invention include, for example, ATP, ?-labeled ATP, cations and co-factors typically utilized by kinases. Solutions useful for performing the method include buffer solutions for controlling or adjusting the pH of the kinase assay mixture, sterile deionized water for diluting and reconstituting reagents, and the like.

The invention is further illustrated by the following non-limiting Examples.

### EXAMPLE 1

### Peptide Synthesis and In Vitro Kinase Assay Materials

DIEA, piperidine (peptide synthesis grade), and TFA (HPLC grade) were obtained from Chem-Impex (Wood Dale, Ill.). DMF, ACN, MTBE, and MeOH were obtained from EM Science (Gibbstown, N.J.). HOBT and HBTU (peptide synthesis grade) were obtained from AnaSpec (San Jose, Calif.). Fmoc-amino acid derivatives were obtained from AnaSpec (San Jose, Calif.) and Chem-Impex (Wood Dale, Ill.). Biotin was obtained from SynPep (Dublin, Calif.).

Peptide Synthesis

Peptides were synthesized as C-terminal amides on Mimotopes (Clayton, Australia) SynPhase Rink amide acrylic-grafted polypropylene solid support (loading 7.5 μmole), arranged in a 12×8 format, in 96 well microtiter plates. Amino acid solution delivery was facilitated by a PinPal Amino Acid Indexer to indicate the appropriate amino acid to be delivered for each peptide in each coupling cycle. A solution containing a mixture of nineteen amino acids was delivered for specific peptides and coupling cycles to create degenerate peptides. Activation was preformed in situ with a solution of 0.1 M HOBT/HBTU/DIEA in DMF. Each unique peptide sequence was synthesized with an N terminal Biotin-Lys-Gly spacer. A dansyl group was attached to the side chain of the spacer Lysine to serve as a chromophore (330 nm) to facilitate peptide quantification. Deprotection with 25% piperidine, DMF and methanol washes were preformed batch wise. After completion of the synthesis, the peptides were cleaved from the solid support and deprotected by acidolysis in the presence of scavengers using TFA/EDT/TA/anisole 90:4:3:3 (v/v/v/v). The crude peptides were precipitated and washed three times with cold MTBE, and lyophilized from water/ACN/HOAc 8:1:1 (v/v/v).

Analysis

The peptide products were validated and quantified via high throughput LC-MS. The system consisted of a Shimadzu (Columbia, Md.) VP series HPLC system and a PE Sciex (Foster City, Calif.) API 165 single quadrapole mass spectrometer. Reverse phase separations of 1 μL injections were preformed using two Phenominex (Torrance, Calif.) 30×1.0 mm Luna 3μ C8 columns at 50° C. with a flow rate of 350 μL/min. The peptides were eluted by a linear gradient from 0% to 60% MeOH (0.1% HOAc) over five minutes and detected at 330 nm and 220 nm. For each LCMS injection, (M+H)/Z was extracted from MS data and compared to the expected mass for that sample, as calculated from its sequence. The UV absorbance trace was integrated to determine purity and yield.

Degenerate Peptide Quantification

Absorbance data for 10 μL aliquots of degenerate peptide solution were acquired using a Labsystems (Beverly, Mass.) Multiskan Ascent plate reader equipped with a 340 nm filter. Yield was determined using a concentration factor calculated from absorbance data acquired on the same system from samples of known concentration that also contained a dansyl chromophore.

Dried degenerate peptides were reconstituted in 90% water/10% ethanol. The concentration of peptide was determined by measurement of absorption at 335 nm (maximal absorption wavelength for dansyl group), stock diluted to 1 mM and stored in sealed well at 4° C. A replica plate was prepared with peptides at 100 μM concentration in 90% water/10% ethanol and stored similarly.

Kinase Preparations

Catalytically active preparations of the kinases of interest were either purchased or prepared. Purchased and tested active kinase preparations including the following: PKC-alpha, PKC-delta, PKC-epsilon, PKC-zeta, PKC-mu, PKA, PKG from Calbiochem, ROK alpha/ROCK-II, active from Upstate Biotechnology, and AKT1 from Panvera.

An example of the purification procedure used for production of active kinase is as follows. A preparation of PKC-theta was prepared using a Gateway expression construct containing PKC-theta that was expressed in baculovirus, which were used to infect Sf9 cells. The cell pellet from a liter of baculovirus-infected Sf9 cells was resuspended in 20 volumes (60 ml) of extraction buffer (20 mM Na phosphate buffer pH 7.5, 500 mM NaCl, 5 mM pyrophosphate, 10% glycerol, 10 mM imidazole, 1 mM PMSF), sonicated twice for one minute (1 cm tip at 60% power and 50% duty cycle) and cell disruption was verified microscopically. The sample was adjusted to five mM MgCl2 and treated with one U benzonase/ml for an additional 20 minute on ice. The sample was clarified by centrifugation in a JA-20 rotor at 15K for 30 min at 4° C., filtered through a 0.8 mm filter and applied at 0.5 ml/min to a one ml chelating sepharose column previously charged with nickel and equilibrated with extraction buffer. The column was washed with extraction buffer at one ml/min to baseline and eluted in a 20 ml gradient (20-500 mM imidazole in extraction buffer) into one ml fractions that were analyzed by SDS-PAGE. Fractions with the highest concentration of protein were pooled, were dialyzed twice against one liter of 20 mM Na PO4 pH 7.5, 50 mM NaCl buffer. The kinase pool was dialyzed twice against 20 mM HEPES pH 7.4, 100 mM NaCl, 2 mM EDTA, 5 mM DTT, 0.05% Triton-X-100. After dialysis, the sample was adjusted to 50% glycerol and quick-frozen in a dry ice/ethanol bath.

More than 20 other preparations of PKC-theta have also been prepared and tested in the inventor's laboratory. The have been typically been transiently expressed in HEK293 cells, and purified by His-tag based isolation conceptually similar to that described above. Alternatively, they were immunoaffinity purified using anti-HA tag antibody to capture the protein when it has been fused to a HA epitope tag; such preps are released by incubation in an excess concentration of HA peptide. These include preparations derived from more than 10 different variant constructs of PKC-theta. Point mutations have been produced using the QuikChange system from Statagene, using the manufacturer's suggested procedures.

Kinase Assay

The conditions of the kinase assay and the amount of active kinase used varied with the kinase and with the accuracy needed. For a typical experiment, 5-20 ng of kinase was used per well and each peptide pool was assayed in duplicate wells. Note that the absolute amount of kinase used was not usually a critical parameter, because the desired information related to specificity of the kinase not its absolute activity, and robustness of the assay depends on comparisons of the same amount of kinase on different peptides. The combination of kinase concentration and assay duration was modified to assure that the stoichiometry of peptide phosphorylation never exceeded 5%. The choice of kinase buffer depended on the kinase being analyzed. For studies of PKC, 100 mM HEPES, 0.05% Triton-X100, 1 mM CaCl2, 20 mM MgCl2, 0.2 mg/ml phosphatidyl serine (Avanti Polar Lipids), PMA 100 ng/ml was typically used. The lipid stock was prepared by transferring 3 mg phosphatidyl serine into iced mixture of 450 μl water plus 50 μl of 10% Triton-X100, sonicating 10 times on ice for 1 sec each.

The kinase reaction mixture was assembled by sequential addition to a tube held on ice of: 5 μl peptide (100 μM for final concentration of 10 μM), 15 μl of kinase (typically 5 ng/well, in appropriate kinase buffer), 30 μl of ATP (1 uCi/well of ³²P-gamma ATP in a stock of 167 μM cold ATP in the kinase buffer; for final concentration for 100 μM ATP). The mixture was rapidly warmed to desired reaction temperature (30° C. for PKC) and incubated for the desired duration (usually 10 minutes). The kinase assay was terminated by transfer to 4° C. water batch, and rapid addition of an equal volume (50 μl) of stop solution [0.1M ATP+0.1M EDTA in water, pH 8].

The peptides were then captured from the reaction mixture by transfer to a Reacti-Bind Streptavidin High Binding Capacity Coated Plates (HBC) (Pierce Biotechnology) as follows. The HBC plates were pre-rinsed three times with PBS/Tween PBS/Tween20 0.05% (PBS/Tween). Part of all of the reaction mixture was then transferred wells of a HBC plate pre-filled with 90 μl of phosphate-buffered saline (PBS); typically each aliquots of each phosphorylation reaction were transferred to duplicate HBC plates to assure accuracy by additional replication

For kinase assays done at the standard peptide concentration of 10 μM, the peptide concentration in the reaction mixture becomes 5 μM after addition of the stop solution; consequently 10 μl of the reaction (50 pMoles of peptide) was transferred to the HBC plate. More generally, the amount of reaction mixture transferred was estimated to be about 50 pMoles of peptide. The inventor had validated that 50 pMoles of peptide was reliably and completely captured by the wells that had a nominal binding capacity of 125 pMoles. The HBC plates were incubated for 0.5 to 1.5 hr at room temperature for complete binding of biotinylated peptides to plate-bound streptavidin. The HBC plates were then washed extensively with PBS/Tween. Five washes were done routinely and additional wash steps were added if the wash solution removed from the plate had measurable radioactivity as detected using a Geiger counter. This step is essential to obtaining a good the signal to noise ratio because the fraction of radioactivity incorporated in the peptides was a tiny fraction of the total in the reaction mixture. The wells were air-dried. A volume of 40-50 μl of microScint-20 (Packard Instruments) was added to each well. The plates were covered with stick-on film sheet. Radioactive emissions were measured in a TopCount NXT Microplate Scintillation and Luminescence Counter (Packard Instruments). Typically samples were counted for 5 minutes (or more) to improve the signal to noise ratio when counts were low.

### EXAMPLE 2

### Use of Reduced Set of Query Residues

The methods described herein provide for systematic variation of the query amino acid between peptides pools of a test set. In one embodiment, all naturally occurring residues will occupy the query amino acid position. In other embodiments, such as illustrated in FIG. 2 and FIG. 6, peptide pool variations at the query position were selected from a reduced set of amino acids.

Because scoring of potential sites in proteins requires a PSSM that includes information on all naturally occurring residues, use of reduced sets requires extrapolation of information from tested residues to residues that have not been tested. The methods of the invention can readily be expanded to include additional residues that provide data to test whether the extrapolated results (e.g. those at the bottom of the chart in FIG. 5) are valid.

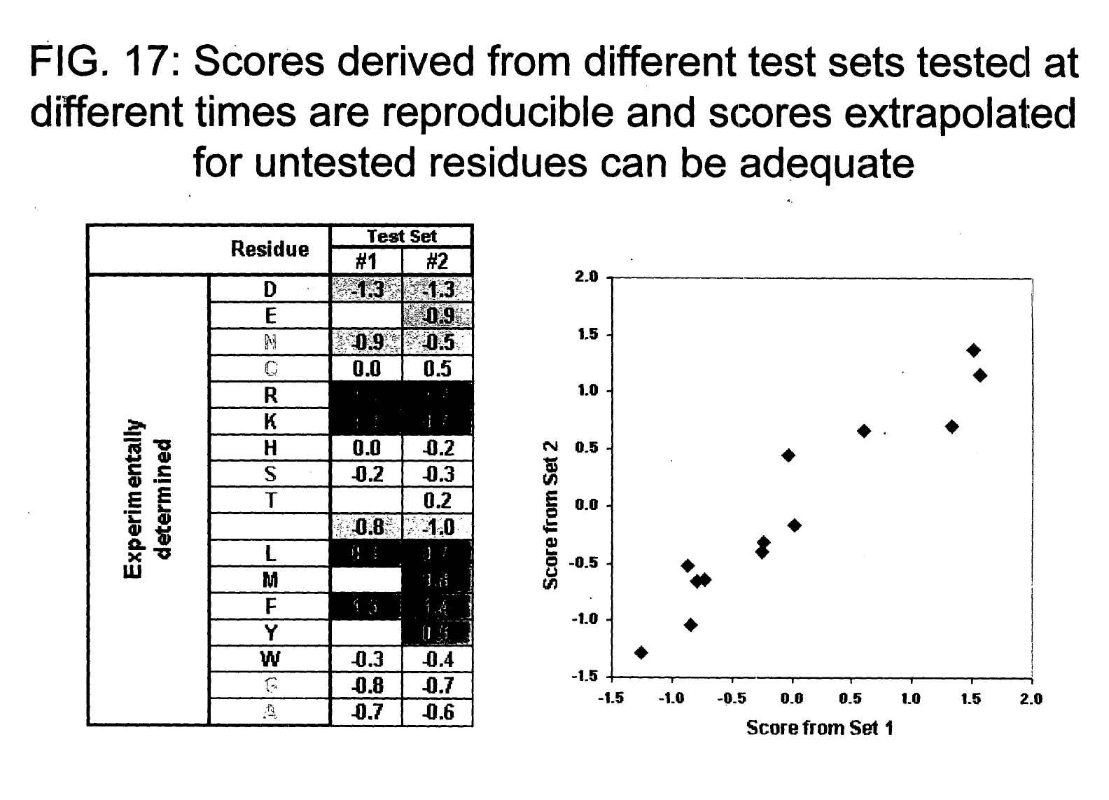

For example, FIG. 17 shows scores for the P+1 position of PKC theta using test set 1 (see also FIG. 2) and a test set 2 that is identical in sequence except that it includes 4 additional query residues and was synthesized several months after test set 1. The two sets were tested in two different experiments that were performed several months apart. Nonetheless, the table and graph in FIG. 17 show that the scores for the residues tested are in very good agreement. The results also showed generally adequate agreement between values extrapolated for untested residues and the values subsequently experimentally determined for those residues. For example, the Log Score for methionine at position P+1 was extrapolated to be 0.7 and experimentally shown to be 0.8. However, the experimentally determined Log Score value for tyrosine (0.5) did differ somewhat from the extrapolated value (1.4). Because the differences in extrapolated and experimentally determined values for tyrosine and phenyalanine were larger than optimal, in preferred embodiments test sets include both F and Y as query residues.

### EXAMPLE 3

### Scoring Phosphorylation Sites Sequences from a PSSM and Predicting Best Phosphorylation Sites

The prior art provides a scoring system by which kinase substrate preferences can be used to make predictions about phosphorylation by the kinase (Yaffe M B, Leparc G G, Lai J, Obata T, Volinia S, Cantley L C. 2001. A motif-based profile scanning approach for genome-wide prediction of signaling pathways. Nat Biotechnol 19:348-353). This example illustrates how that scoring approach is done and validates the methods described herein when applied to a known PKC substrate.

Methods Employed

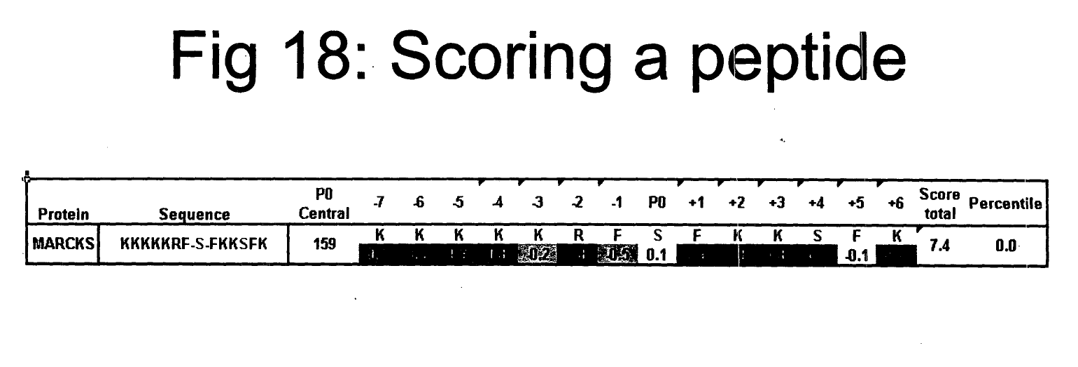

As shown in FIG. 18, a raw total score can readily be calculated for any peptide sequence using the data in a PSSM, for example, the PSSMs provided in FIG. 5, FIG. 7, and FIG. 17. The total score was determined by adding together the PSSM score for each of the residues of the peptide. This type of calculation is illustrated in FIG. 18 for a peptide corresponding to a known PKC phosphorylation site in the protein MARCKS having the sequence KKKKKRF-S-FKKSFK (SEQ ID NO:474). The score derived was for the sequence surrounding the Ser-159 of the intact MARCKS protein. For example, because the P−7 position of MARCKS was occupied by K, a score of 0.4 from column P−7 of FIG. 7 was used. The scores for the other thirteen residues were similarly derived from columns of FIG. 5, FIG. 7, and FIG. 17. The fourteen scores were combined for a total score of 7.4 for the KKKKKRF-S-FKKSFK (SEQ ID NO:474) sequence in MARCKS.

The raw total scores are informative in ranking individual peptides. However, it was even more useful to estimate the relative likelihood of phosphorylation of a peptide compared to many other peptides in the human proteome (i.e. proteins encoded by human genes). Such an estimate can be conveniently represented by a percentile score. To convert a raw score for a peptide to a percentile score, a relevant set of peptide scores must first be collected and sorted. Then, the relative position of the raw total score within that ordered set is determined.

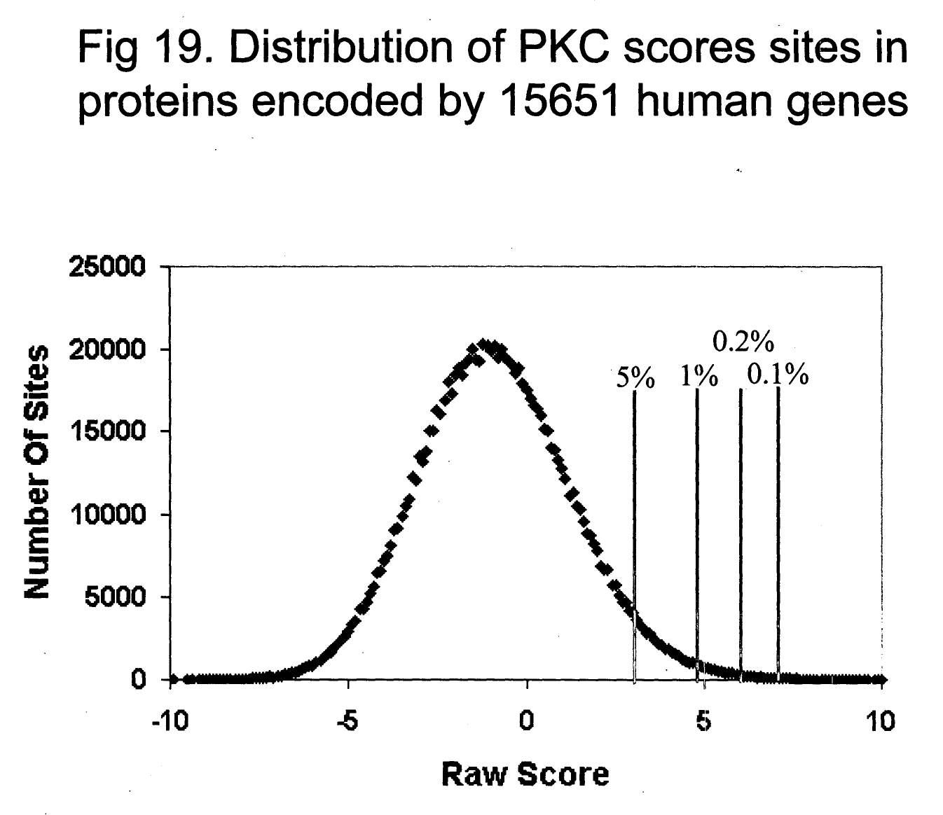

Peptide sequences were examined that surrounded 1,071,932 Ser and Thr residues found in proteins encoded by 15651 human genes catalogued in the human reference sequence (RefSeq) collection maintained by the National Center for Biotechnology Information. The sequence of each protein was scanned to identity each residue that could be phosphorylated on Ser or Thr. The sequence surrounding each of these sites was used to calculate a raw score for that site for each PSSM. The distribution of scores was determined, as illustrated, for example, in FIG. 19 for the PKC-theta PSSM. The median score for all these proteins was −0.9.

From this distribution, a percentile score was determined for any given raw score. For example, a raw score of >2.8 corresponds to the top 5 percentile and a raw score of >6.2 corresponds to the top 0.2 percentile of sites likely to be phosphorylated by a selected kinase. Using this distribution, each score can be assigned a percentile. For example, a raw score of 7.4 for the KKKKKRF-S-FKKSFK (SEQ ID NO:474) sequence in MARCKS corresponds to the 0.04 percentile. Such a low percentile indicates that the KKKKKRF-S-FKKSFK (SEQ ID NO:474) sequence in MARCKS is amongst the best candidate substrates for PKC. Therefore, this kind of finding indicates that using the PSSM provided by FIG. 5, FIG. 7, and FIG. 17, one of skill in the art can predict which sequence within which protein is particularly likely be phosphorylated by PKC-theta.

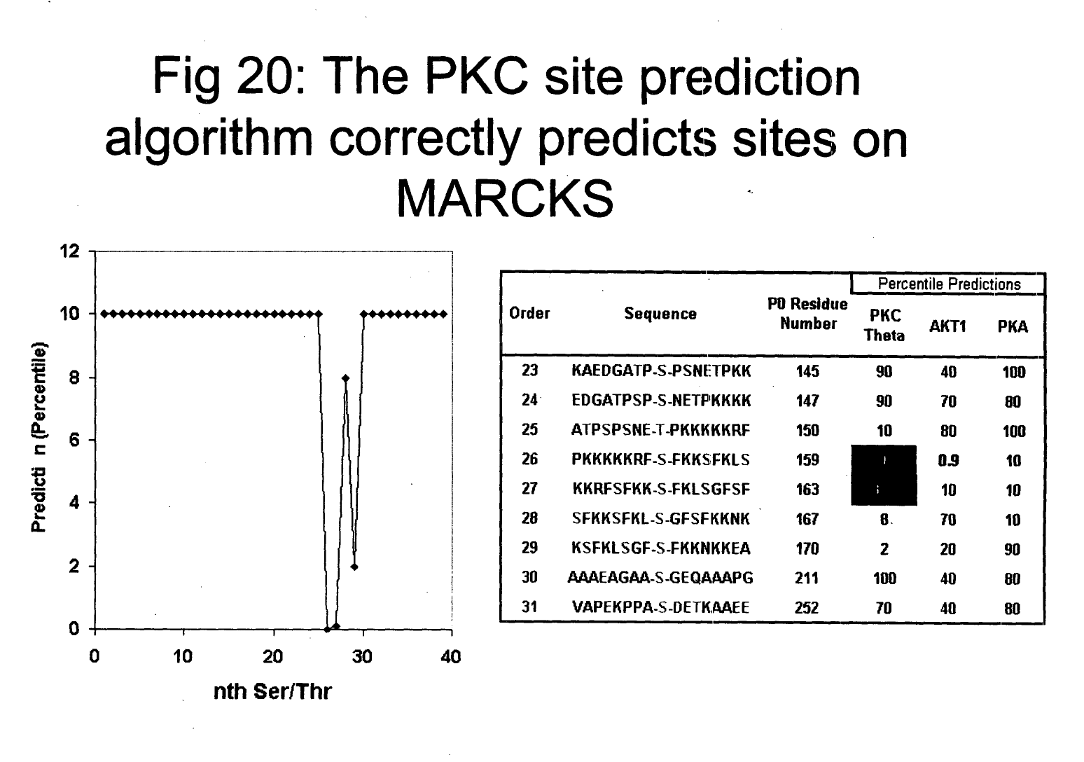

In another embodiment, the invention provides methods for identifying which sites in a protein of interest are likely to be phosphorylated by a particular kinase, such as PKC-theta. FIG. 20 illustrates such an analysis for the thirty nine Ser and Thr residues in the protein MARCKS. The panel on the left shows the percentile score for each of the thirty nine residues. There is only one region of the MARCKS protein in which PKC phosphorylation sites are likely located. The panel on the right shows a portion of the analysis corresponding to this most likely region. Each row shows a candidate site, together with information on the position of the candidate site, and percentile predictions for phosphorylation at the candidate position by three kinases studied: PKC-theta, AKT1, and PKA. As shown in FIG. 20, two very strong candidate sites exist for PKC-theta at P0 positions 159 and 163 (percentile <0.2). The values for AKT1 and PKA suggest there are much less likely to be sites for phosphorylation by those kinases. These sites are precisely the two sites known to be physiologically relevant PKC phosphorylation sites in MARCKS. This kind of validation has been reproduced in a number of other molecules with known PKC phosphorylation sites, such as alpha-, beta-, gamma-adducins, and GAP-43.

### EXAMPLE 4

### Identification of In Vitro Phosphorylation Sites for PKC

Many peptides that are good substrates for PKC enzymes were identified using the methods of the invention. For example, Tables 4 and 5 provide a listing of peptides identified as potentially useful kinase substrates. The locuslink identifier (NCBI) for the gene, the gene symbol and the peptide sequence, together with results for results for phosphorylation by up to seven different kinases are provided Tables 4 and 5. Five PKC isoforms were tested using the methods described herein (see, e.g. Example 1): one classical PKC isoform (PKC-alpha), three “novel” PKC isoforms (PKC-epsilon, PKC-delta and PKC-theta) and one atypical PKC isoform (PKC-zeta). The data provided in Tables 4 and 5 show that novel and classical PKCs exhibit similar phosphorylation site preferences. In contrast to the general similarity of the substrates selected by the four classical PKC isoforms tested (PKC-alpha, PKC-epsilon, PKC-delta and PKC-theta), a more distant PKC isoform (PKC-zeta) and two other kinases in the same superfamily (AGC) show rather different patterns of phosphorylation. Note that Table 5 includes data for two different concentrations of substrate peptide during the assay (10 μM and 1 μM). Results are substantially similar at those two concentrations, indicating that these findings on specificity are of general relevance and pertain to phosphorylation over a broad range of substrate concentrations.

TABLE 4

Identification of additional PKC substrates
PKC isoforms alpha, epsilon, delta and theta have similar specificity.

| | | | | | | | | | | | | |
|---|---|---|---|---|---|---|---|---|---|---|---|---|
| | | | SEQ | | | | | | | | | |
| | Name or | | ID | | | PKC | PKC- | PKC | PKC | PKC- | | |
| LocusLink | Gene Symbol | Position | NO: | Sequence | average | alpha | epsilon | delta | theta | zeta | PKA | AKT1 |

| | | | | | | | | | | | | |
|---|---|---|---|---|---|---|---|---|---|---|---|---|
| 4296 | MLK3 | 477 | 76 | HVRRRRGTFKRSKLRARD | 91 | 62 | 100 | 100 | 100 | 41 | 69 | 100 |
| 8525 | DGKZ | 265 | 77 | KKKKRASFKRKSSKKG | 80 | 100 | 71 | 76 | 74 | 60 | 4 | 8 |
| 5341 | PLEK | 0 | 78 | KFARKSTRRSIRLPE | 63 | 69 | 84 | 52 | 47 | 100 | 5 | 2 |
| 9162 | DGKI | 345 | 79 | NRKKKRTSFKRKA | 60 | 50 | 92 | 66 | 33 | 18 | 3 | 7 |
| 4082 | MARCKS | 159 | 80 | KKKKKRFSFKKSFKL | 56 | 65 | 71 | 24 | 63 | 16 | 3 | 6 |
| 5339 | PLEC1 | 0 | 81 | KRERKTSSKSSVRKRR | 56 | 62 | 59 | | 46 | 10 | 2 | 9 |
| 9828 | p164-RhoGEF | 369 | 82 | PRLIRRGSKKRPAR | 56 | 61 | 72 | 40 | 49 | 38 | 10 | 9 |
| 1128 | CHRM1 | 451 | 83 | RKIPKRPGSVHRTPSRQ | 55 | 41 | 47 | 38 | 92 | 4 | 2 | 6 |
| 2561 | GABRB2 | 472 | 84 | QKKSRLRRRASQLKI | 53 | 39 | 52 | 34 | 87 | 19 | 63 | 10 |
| 5578 | PRKCA | 25 | 85 | RFARKGSLRQKNV | 52 | 42 | 86 | 34 | 46 | 56 | 81 | 10 |
| 3757 | KCNH2 | 890 | 86 | RQRKRKLSFRRRTDKD | 48 | 47 | 63 | 42 | 38 | 28 | 87 | 39 |
| 94121 | SYTL4 | 414 | 87 | RQGKRKTSIKRDTVNPL | 47 | 46 | 31 | | 65 | 3 | 64 | 7 |
| 65108 | MACMARCKS | 104 | 88 | KKPFKLSGLSFKRNRKE | 43 | 48 | 44 | | 38 | 4 | 3 | 10 |
| 55357 | PARIS1 | 449 | 89 | EYLERRASRRRAV | 41 | 37 | 45 | 20 | 60 | 14 | 4 | 1 |
| 286 | ANK1 | 68 | 90 | AQIVKRASLKRGKQ | 40 | 40 | 49 | 32 | 38 | 5 | 3 | 3 |
| 5587 | PRKCM | 437 | 91 | VHYTSKDTLRKRHYWR | 40 | 60 | 49 | | 10 | 12 | 3 | 5 |
| 395 | ARHGAP6 | 257 | 92 | DGQKRKKSLRKKLD | 38 | 36 | 51 | 17 | 47 | 2 | 18 | 2 |
| 9266 | PSCD2 | 392 | 93 | AARKKRISVKKKQEQ | 37 | 34 | 40 | 35 | 40 | 1 | 1 | 7 |
| 2081 | ERN1 | 724 | 94 | KLAVGRHSFSRRSGV | 36 | 34 | 13 | 12 | 86 | 10 | 5 | 9 |
| 119 | ADD2 | 713 | 95 | KKKFRTPSFLKKSKK | 33 | 34 | 31 | 25 | 42 | 5 | 2 | 9 |
| 775 | CACNA1C | 1898 | 96 | RGFLRSASLGRRASFHLE | 33 | 40 | 41 | 18 | 21 | 22 | 86 | 32 |
| 434 | ASIP | 78 | 97 | KKRSSKKEASMKKVVRP | 28 | 37 | 16 | | 31 | 1 | 1 | 10 |
| 393 | ARHGAP4 | 217 | 98 | AGPLRKSSLKKGGRL | 27 | 25 | 34 | | 22 | 4 | 21 | 7 |
| 9590 | AKAP12 | 311 | 99 | AGWRKKTSFRKPKED | 27 | 37 | 27 | 17 | 25 | 10 | 1 | 6 |
| 9020 | MAP3K14 | 140 | 100 | WKGKRRSKARKKRK | 26 | 14 | 13 | 22 | 56 | 8 | 2 | 3 |
| 4687 | NCF1 | 0 | 101 | GAPPRRSSIRNAH | 23 | 32 | 19 | 13 | 29 | 3 | 100 | 12 |
| 4763 | NF1 | 2813 | 102 | AGSFKRNSIKKIV | 23 | 35 | 14 | 14 | 28 | 2 | 5 | 6 |
| 4607 | MYBPC3 | 0 | 103 | LLKKRDSFRTPRDSKLE | 22 | 32 | 21 | 12 | 24 | 8 | 3 | 7 |
| 8436 | SDPR | 235 | 104 | EKIKRSSLKKVDSLKK | 22 | 34 | 17 | 10 | 25 | 3 | 1 | 2 |
| 9148 | NEURL | 238 | 105 | ALRRPSLRREADD | 21 | 26 | 29 | 9 | 21 | 4 | 84 | 12 |
| 1385 | CREB1 | 0 | 106 | EILSRRPSYRKILND | 21 | 23 | 31 | 9 | 19 | 15 | 30 | 12 |
| 94274 | PPP1R14A | 38 | 107 | QKRHARVTVKYDRRE | 19 | 18 | 7 | 10 | 40 | 1 | 2 | 3 |
| 3985 | LIMK2 | 473 | 108 | KATTKKRTLRKNDRK | 16 | 19 | 8 | 6 | 32 | 1 | 0 | 1 |
| 8013 | NR4A3 | 366 | 109 | KEVVRTDSLKGRRGR | 16 | 21 | 26 | 7 | 9 | 2 | 1 | 2 |
| 57731 | SPTBN4 | 2555 | 110 | EGGDRRASGRRK | 15 | 30 | 4 | 5 | 22 | 3 | 1 | 1 |
| | MARCKS | | 111 | KKKRFSFKKSFKLSGFSFKK | 14 | 11 | 12 | 16 | 18 | 9 | 3 | 3 |
| 10969 | EBNA1BP2 | 289 | 112 | KRPGKKGSNKRPGKR | 12 | 10 | 11 | 8 | 17 | 1 | 0 | 3 |
| 54986 | FLJ20574 | 174 | 113 | GENVLKKSMKSRVKG | 10 | 16 | 10 | 4 | 10 | 1 | 0 | 9 |

TABLE 5

Identification of additional substrates for PKC
Specificities of PKC-theta, -delta, -epsilon and -alpha are similar

| | | | | | | | |
|---|---|---|---|---|---|---|---|
| | | average | | | | | |
| | | Peptide | | | | | |
| SEQ | | concen- | PKC | PKC- | | | |
| ID | Locus | tration | theta | delta | epsilon | alpha | zeta |

| | | | | | | | | | | | | | | |
|---|---|---|---|---|---|---|---|---|---|---|---|---|---|---|
| Sequence | NO: | Link ID | Std Name | P0Range | (uM) | 10 | 1 | 10 | 1 | 1 | 10 | 1 | 10 | 1 |

| | | | | | | | | | | | | | | |
|---|---|---|---|---|---|---|---|---|---|---|---|---|---|---|
| KKKKRASFKRKSSKKG | 114 | 8525; | dag kinase zeta | 265-265; | 95 | 86 | 100 | 100 | 80 | 100 | 100 | 100 | 51 | 45 |
| NRKKKRTSFKRKA | 115 | 9162; | dag kinase iota | 344-344; | 80 | 77 | 89 | 99 | 94 | 91 | 56 | 55 | 10 | 11 |
| EEGTFRSSIRRLSTRRR | 116 | 5348; | phospholemman | 79-79; | 71 | 78 | 83 | 41 | 75 | 92 | 57 | 70 | 100 | 100 |
| NRKKKRTSFKRKA | 117 | 9162; | dag kinase iota | 344-344; | 70 | 78 | 84 | 79 | 100 | 67 | 39 | 40 | 11 | 20 |
| RPQNTLKASKKKKRASFKRK | 118 | 8525; | dag kinase zeta | 254-254; | 64 | 52 | 67 | 60 | 73 | 89 | 60 | 46 | 57 | 46 |
| KKRFSFKKSFKLSGFSFKKN | 119 | 4082; | MARCKS | 159-159; | 61 | 28 | 69 | 16 | 42 | 81 | 98 | 94 | 20 | 20 |
| AKRRRLSSLRASTSK | 120 | 6194; | ribosomal protein | 235-235; | 52 | 51 | 51 | 32 | 64 | 56 | 58 | 55 | 34 | 43 |
| | | | S6 | | | | | | | | | | | |
| LRRRSLRRSNSISKSPGP | 121 | 3985; | LIMK-2 | 283-283; | 50 | 57 | 51 | 53 | 80 | 49 | 29 | 33 | 56 | 60 |
| | | | | 262-262; | | | | | | | | | | |
| RAITSTLASSFKRRR | 122 | 2902; | NMDAR1 | 884-884; | 49 | 42 | 66 | 24 | 55 | 72 | 44 | 43 | 17 | 20 |
| KKRFSFKKSFKLSGFSFKKN | 123 | 4082; | MARCKS | 159-159; | 49 | 32 | 47 | 22 | 45 | 67 | 73 | 58 | 20 | 20 |
| PRLIRRGSKKRPAR | 124 | 9828; | p164-RhoGEF | 922-922; | 49 | 57 | 53 | 43 | 69 | 49 | 24 | 48 | 32 | 45 |
| PLKEKKRERKTSSKSSVRKR | 125 | 5339; | plectin | 4157-4157; | 48 | 100 | 39 | 36 | 46 | 48 | 31 | 34 | 12 | 13 |
| KAIKAIEGGQKFARKSTRRS | 126 | 5341; | pleckstrin 1 | 113-113; | 46 | 45 | 46 | 43 | 47 | 69 | 34 | 36 | 56 | 46 |
| SQVQKQRSAGSFKRNSIKKI | 127 | 4763; | NF1 | 2798-2798; | 43 | 58 | 45 | 33 | 52 | 32 | 39 | 40 | 19 | 19 |
| QQVDRERPHVRRRRGTFKRS | 128 | 4296; | MLK3 | 477-477; | 42 | 59 | 41 | 57 | 46 | 32 | 33 | 25 | 9 | 14 |
| VQRHRSMRKTFARYLSFRRD | 129 | 4171; | MCM2; | 801-801; | 40 | 25 | 48 | 14 | 32 | 65 | 37 | 58 | 31 | 51 |
| EYLERRASRRRAV | 130 | 55357; | PARIS1 | 443-443; | 39 | 44 | 38 | 20 | 46 | 46 | 38 | 38 | 39 | 36 |
| WKGKRRSKARKKRK | 131 | 9020; | NIK | 140-140; | 38 | 35 | 53 | 29 | 38 | 43 | 22 | 49 | 6 | 7 |
| GFLNEPLSSKSQRRKSLKLK | 132 | 57082; | AF15q14; | 1059-1059; | 38 | 40 | 34 | 47 | 41 | 47 | 30 | 24 | 61 | 19 |
| | | | | 1059-1059; | | | | | | | | | | |
| | | | | 1085-1085; | | | | | | | | | | |
| LEKRGMLGKRPRRKSSRRKK | 133 | 3797; | kinesin 3C | 408-408; | 37 | 53 | 46 | 52 | 46 | 31 | 16 | 17 | 30 | 27 |
| RSRSRSRSKSKDKRKSRKRS | 134 | 6429; | splicing factor, | 342-342; | 34 | 16 | 36 | 16 | 44 | 32 | 39 | 54 | 5 | 5 |
| | | | arginine/serine- | | | | | | | | | | | |
| | | | rich 4 | | | | | | | | | | | |
| KKKFRTPSFLKKSKK | 135 | 119; | beta adducin | 711-711; | 33 | 36 | 30 | 25 | 40 | 27 | 19 | 56 | 13 | 11 |
| RARRDSLKKIEIW | 136 | 9101; | ubiquitin specific | 994-994; | 32 | 28 | 42 | 23 | 41 | 33 | 28 | 26 | 9 | 6 |
| | | | protease 8 | | | | | | | | | | | |
| PSKSPSKKKKKFRTPSFLKK | 137 | 119: | beta adducin | 699-699; | 31 | 19 | 40 | 16 | 38 | 32 | 38 | 34 | 5 | 4 |
| EYLERRASRRRAV | 138 | 55357; | PARIS1 | 443-443; | 31 | 39 | 34 | 28 | 49 | 27 | 20 | 17 | 61 | 31 |
| RPTPGDGEKRSRIKKSKKRK | 139 | 79142; | MGC2941; | 205-205; | 30 | 17 | 31 | 24 | 31 | 24 | 44 | 39 | 1 | 1 |
| TELEGGFSRQRKRKLSFRRR | 140 | 3757; | HERG | 875-875; | 30 | 35 | 25 | 49 | 44 | 27 | 15 | 12 | 49 | 24 |
| VTDSQKRREILSRRPSYPKI | 141 | 1385; | CREB | 105-105; | 29 | 28 | 29 | 21 | 43 | 31 | 25 | 24 | 78 | 26 |
| ERHVAQKKSRLRRRASQLKI | 142 | 2561; | GABA A receptor | 465-465; | 28 | 37 | 28 | 36 | 30 | 28 | 21 | 16 | 37 | 22 |
| | | | beta 2 | | | | | | | | | | | |
| VRYTPYTISPYNRKGSFRKQ | 143 | 56000; | nuclear RNA export | 66-66; | 28 | 27 | 25 | 32 | 33 | 21 | 19 | 38 | 79 | 22 |
| | | | factor 3 | | | | | | | | | | | |
| LSSMFGTLPRKSRKGSVRKQ | 144 | 9595; | PSCDBP | 322-322; | 28 | 22 | 35 | 28 | 35 | 24 | 20 | 31 | 54 | 16 |
| ISDFGLAKKLAVGRHSFSRR | 145 | 2081; | ERN1 | 710-710; | 27 | 25 | 27 | 23 | 34 | 26 | 30 | 21 | 61 | 25 |
| QAQRQIKRGAPPRRSSIRNA | 146 | 4687; | p47phox | 303-303; | 27 | 34 | 33 | 14 | 33 | 28 | 24 | 20 | 56 | 32 |
| RDIRQSPKRGFLRSASLGRR | 147 | 775; | calcium channel, | 1924-1924; | 26 | 69 | 20 | 13 | 27 | 26 | 16 | 13 | 34 | 28 |
| | | | voltage-dependent, | | | | | | | | | | | |
| | | | L type, alpha | | | | | | | | | | | |
| RELEQLKAEYLERRASRRRA | 148 | 55357; | PARIS1 | 443-443; | 26 | 34 | 34 | 14 | 27 | 33 | 22 | 19 | 20 | 20 |
| RVVQSVKHTKRKSSTVMK | 149 | 5587; | PKD1 | 412-412; | 25 | 14 | 13 | 17 | 20 | 27 | 52 | 33 | 5 | 3 |
| VDPFYEMLAARKKRISVKKK | 150 | 9266; | cytohesin-2 | 381-381; | 24 | 35 | 35 | 20 | 34 | 20 | 11 | 12 | 6 | 3 |
| PQNSLKASNRKKKRTSFKRK | 151 | 9162; | dag kinase iota | 333-333; | 24 | 19 | 25 | 37 | 27 | 24 | 16 | 17 | 28 | 15 |
| DLIEGRKGAQIVKRASLKRG | 152 | 286; | ankyrin R | 68-68; | 23 | 30 | 31 | 18 | 35 | 25 | 12 | 13 | 9 | 4 |
| TYLLPDKSRQGKRKTSIKRD | 153 | 94121; | slp4 | 399-399; | 23 | 18 | 22 | 26 | 32 | 24 | 22 | 17 | 13 | 6 |
| KKFFTQGWAGWRKKTSFRKP | 154 | 9590; | gravin | 301-301; | 22 | 21 | 34 | 14 | 26 | 29 | 12 | 16 | 23 | 27 |
| | | | | 203-203; | | | | | | | | | | |
| RWDKRRWRKIPKRPGSVHRT | 155 | 1128; | M1 muscarinic | 451-451; | 21 | 26 | 23 | 23 | 30 | 26 | 12 | 10 | 6 | 19 |
| | | | receptor | | | | | | | | | | | |
| SAQITIPKDGQKRKKSLRKK | 156 | 395; | ARHGAP6 | 242-242; | 20 | 23 | 18 | 31 | 29 | 18 | 11 | 9 | 44 | 15 |
| PSPSNETPKKKKKRFSFKKS | 157 | 4082; | MARCKS | 145-145; | 20 | 23 | 20 | 35 | 27 | 12 | 11 | 10 | 6 | 3 |
| VQMTWSYPDEKNKRASVRRR | 158 | 2321; | fit1 | 265-265; | 19 | 23 | 19 | 33 | 18 | 11 | 11 | 16 | 72 | 11 |
| LYARLARAYRRSQRASFKRA | 159 | 2837; | Urotensin-2 | 231-231; | 19 | 17 | 21 | 12 | 15 | 25 | 19 | 21 | 41 | 25 |
| | | | receptor | | | | | | | | | | | |
| PFEVVWYKDKRQLRSSKKYK | 160 | 7273; | titin | 6478-6478; | 18 | 25 | 19 | 31 | 25 | 13 | 7 | 9 | 57 | 15 |
| KYKAFIRIPIPTRRHTFRRQ | 161 | 5337; | PLD1 | 133-133; | 18 | 23 | 19 | 14 | 22 | 21 | 8 | 22 | 9 | 15 |
| KKKFSFKKPFKLSGLSFKRN | 162 | 65108; | MacMARCKS | 93-93; | 17 | 13 | 14 | 13 | 21 | 19 | 19 | 17 | 4 | 7 |
| PPRTPGWHQLQPRRVSFRGE | 163 | 9088; | Myt1 kinase | 71-71; | 16 | 11 | 14 | 21 | 13 | 14 | 10 | 30 | 26 | 7 |
| TEGKMARVAWKGKRRSKARK | 164 | 9020; | NIK | 125-125; | 16 | 20 | 24 | 26 | 18 | 12 | 6 | 6 | 2 | 3 |
| TEEKSKKRKKKHRKNSRKHK | 165 | 9360; | cyclophilin G | 223-223; | 16 | 14 | 16 | 19 | 25 | 12 | 10 | 15 | 2 | 1 |
| MAQIERGEARIQRRISIKKA | 166 | 6594; | SMARCA5 | 931-931; | 15 | 13 | 13 | 29 | 13 | 10 | 7 | 22 | 9 | 2 |
| | | 8467; | | 910-910; | | | | | | | | | | |
| | | | | 916-916; | | | | | | | | | | |
| GLPAPGEDKSIYRRGSRRWR | 167 | 5590; | pkc-zeta | 113-113; | 15 | 14 | 12 | 24 | 13 | 6 | 7 | 27 | 37 | 5 |
| | | | | 113-113; | | | | | | | | | | |

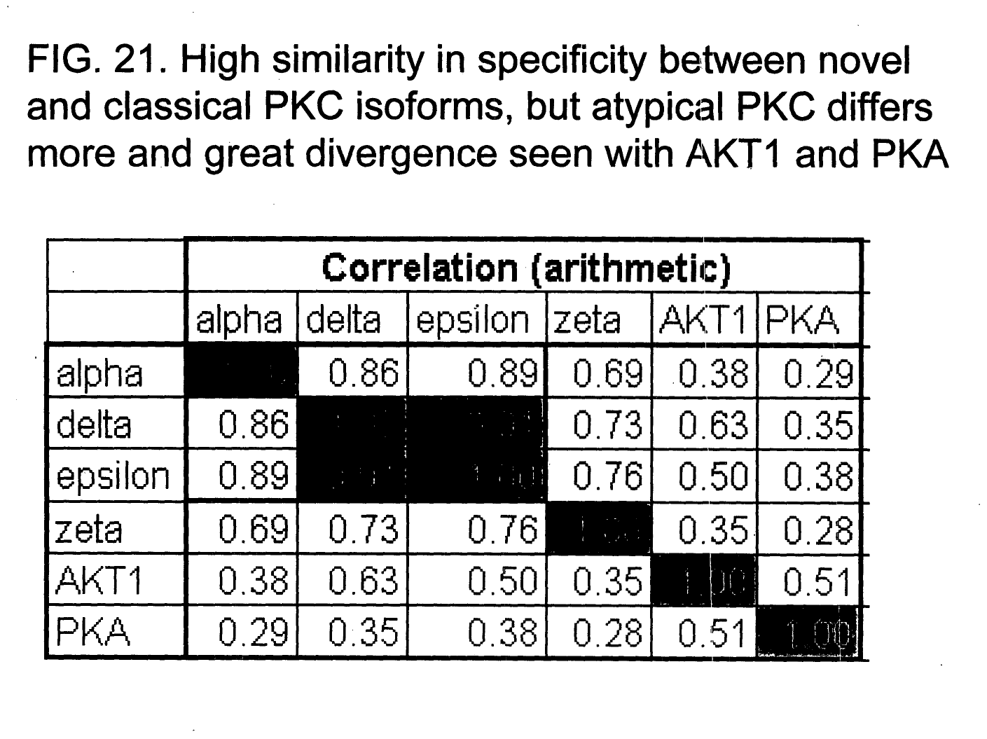

Quantitative analysis of correlations between phosphorylation of the same substrate by different kinases is shown in FIG. 21. Such analysis confirms the conclusions that the novel and classical PKC isoforms are very similar in specificity, that there is greater divergence of the atypical PKC isoform PKC-zeta, and that the other kinases of the same superfamily (AGC) are even more divergent in specificity.

Results in Table 2, Table 3, Table 4 and Table 5 demonstrate phosphorylation by PKC of many of the peptides. As validated herein, the methods of the invention predict that Ser and Thr residues within those peptides are the preferred sites of phosphorylation. Table 6 lists sequences of peptides in which pSer and pThr are present at positions corresponding to preferred PKC phosphorylation sites in peptides phosphorylated by PKC. Phosphopeptides included in Table 6 are only those corresponding to peptides whose efficiency of phosphorylation by PKC is greater than or equal to 10% of the best substrate. Such a cutoff is relatively stringent. It is more rigorous than many previous methods in which the magnitude of phosphorylation is not compared with reference positives.

TABLE 6

Sequence of phosphopeptides
corresponding to preferred sites of PKC
phosphorylation provided by the invention

| | | | | Percentile |
|---|---|---|---|---|
| SEQ | Locus- | | Sequence | prediction |
| ID | Link | | indicating site | for PKC- |
| NO | ID | Name | of phospharylation | theta |
| 301 | 202 | absent in | RSGRRRG-pS-QKSTDS | 0.0 |
| | | melanoma 1 | | |
| 302 | 286 | ankyrin R | AQIVKRA-pS-LKRGKQ | 0.3 |
| 303 | 695 | BTK | FERGRRG-pS-KKGSID | 0.2 |
| 304 | 1105 | CHD1 | SEGRRSR-pS-RRYSGS | 0.1 |
| 305 | 1455 | casein | FKRRKRK-pS-LQRHK- | 0.1 |
| | | kinase I | | |
| | | gamma 2 | | |
| 306 | 1612 | DAP-kinase | IKKRRTK-pS-SRRGVS | 0.0 |
| | | 1 | | |
| 307 | 1612 | DAP-kinase | KKRRTKS-pS-RRGVSR | 0.2 |
| | | 1 | | |
| 308 | 1794 | DOCK2 | PEVKLRR-pS-KKRTKR | 0.1 |
| 309 | 1901 | S1P1 | YSLVRTR-pS-RRLTFR | 0.1 |
| | | receptor | | |
| 310 | 2870 | GRK6 | GGNRKGK-pS-KKWRQM | 0.5 |
| 311 | 3985 | LIMK-2 | ---LRRR-pS-LRRSNS | 0.0 |
| 312 | 4033 | JAW1 | RFSRRSS-pS-WRILGS | 0.6 |
| 313 | 4296 | MLK3 | RRGTFKR-pS-KLRARD | 0.8 |
| 314 | 4296 | MLK3 | HVRRRRG-pT-FKRSKL | 0.0 |
| 315 | 4542 | myosin IF | KKERRRN-pS-INRNFV | 0.0 |
| 316 | 4820 | NKTR | TSSYRSR-pS-YSRSRS | 0.7 |
| 317 | 5128 | PCTK2 | KKFKRRL-pS-LTLRGS | 0.1 |
| 318 | 5339 | plectin | RKTSSKS-pS-VRKRR- | 0.5 |
| 319 | 5734 | prosta- | SDFRRRR-pS-FRRIAG | 0.0 |
| | | glandin E | | |
| | | receptor 4 | | |
| 320 | 5777 | SHP-1 | DKEKSKG-pS-LKRK-- | 2.0 |
| 321 | 5778 | HePTP | RALSFRQ-pT-SWLS-- | 2.0 |
| 322 | 5778 | HePTP | EQQRRAL-pS-FRQTSW | 3.0 |
| 323 | 7074 | TIAM1 | QAMSRSA-pS-KRRSRF | 0.6 |
| 324 | 9221 | Nucl. pp130 | KTKKKRG-pS-YRGGSI | 0.5 |
| 325 | 9266 | cytohesin-2 | AARKKRI-pS-VKKKQE | 0.2 |
| 326 | 9360 | cyclophilin | KKKHRKN-pS-RKHK-- | 0.0 |
| | | G | | |
| 327 | 9595 | PSCDBP | FGTLPRK-pS-RKGSVR | 0.2 |
| 328 | 9595 | PSCDBP | PRKSRKG-pS-VRKQ-- | 0.0 |
| 329 | 9595 | PSCDBP | SSSRRNR-pS-ISN--- | 0.3 |
| 330 | 9595 | PSCDBP | DFLRRSS-pS-RRNRSI | 0.3 |
| 331 | 23031 | MAST3 | --RMARR-pS-KRSRRR | 0.2 |
| 332 | 23031 | MAST3 | ETQDRRK-pS-LFKKIS | 0.4 |
| 333 | 23031 | MAST3 | MARRSKR-pS-RRRETQ | 0.2 |
| 334 | 25836 | IDN3 | RRRSQRI-pS-QRIT-- | 0.0 |
| 335 | 25836 | IDN3 | SGVRRRR-pS-QRISQR | 0.0 |
| 336 | 26191 | Lyp | VILRPSK-pS-VKLRSP | 0.6 |
| 337 | 65125 | WNK1 | RRRRPTK-pS-KGSKSS | 1.0 |
| 338 | 65125 | WNK1 | SGRRRRP-pT-KSKGSK | 0.0 |
| 339 | 65125 | WNK1 | RKSVRSR-pS-RHE--- | 0.6 |
| 340 | 65125 | WNK1 | TKRHYRK-pS-VRSRSR | 0.0 |
| 341 | 393 | ARHGAP4 | AGPLRKS-pS-LKKGGR | 0.3 |
| 342 | 409 | beta- | EKSHKRN-pS-VRLVIR | 0.5 |
| | | arrestin2 | | |
| 343 | 119 | adducin | TPSFLKK-pS-KK---- | 2.0 |
| | | gamma | | |
| 344 | 202 | absent in | -----RR-pS-GRRRGS | 0.5 |
| | | melanoma 1 | | |
| 345 | 395 | ARHGAP6 | DGQKRKK-pS-LRKKLD | 0.1 |
| 346 | 672 | BRCA1 | NRLRRKS-pS-TRHIHA | 0.1 |
| 347 | 672 | BRCA1 | -NRLRRK-pS-STRHIH | 0.1 |
| 348 | 775 | calcium | RGFLRSA-pS-LGRR-- | 1.0 |
| | | channel, | | |
| | | voltage | | |
| | | dependent, | | |
| | | L type, | | |
| | | alpha | | |
| 349 | 1105 | CHD1 | -GSEGRR-pS-RSRRYS | 0.7 |
| 350 | 1196 | CLK2 | RRRRRSR-pT-FSRSSS | 0.0 |
| 351 | 1196 | CLK2 | RRRSRTF-pS-RSSS-- | 1.0 |
| 352 | 1198 | CLK3 | YRWKRRR-pS-YSREHE | 0.1 |
| 353 | 1794 | DOCK2 | LRRSKKR-pT-KRSS-- | 0.9 |
| 354 | 2081 | ERN1 | KLAVGRH-pS-FSRRSG | 1.0 |
| 355 | 2081 | ERN1 | AVGRHSF-pS-RR---- | 4.0 |
| 356 | 2305 | forkhead- | -RERRER-pS-RSRRKQ | 0.0 |
| | | like 16 | | |
| 357 | 2305 | forkhead- | ERRERSR-pS-RRKQHL | 0.4 |
| | | like 16 | | |
| 358 | 3797 | kinesin 3C | KRPRRKS-pS-RRKK-- | 0.0 |
| 359 | 3797 | kinesin 3C | GKRPRRK-pS-SRRKK- | 0.0 |
| 360 | 3985 | LIMK-2 | KATTKKR-pT-LRKNDR | 1.0 |
| 361 | 3985 | LIMK-2 | RRRSLRR-pS-NSISKS | 0.5 |
| 362 | 3985 | LIMK-2 | RSLRRSN-pS-ISKSPG | 0.1 |
| 363 | 4033 | JAW1 | DRFSRRS-pS-SWRILG | 3.0 |
| 364 | 4033 | JAW1 | -DRFSRR-pS-SSWRIL | 3.0 |
| 365 | 4171 | MCM2; | --VQRHR-pS-MRKTFA | 0.0 |
| 366 | 4763 | NF1 | AGSFKRN-pS-IKKIV- | 0.5 |
| 367 | 4820 | NKTR | SYRSRSY-pS-RSRSRG | 2.0 |
| 368 | 4820 | NKTR | RSRSYSR-pS-RSRG-- | 1.0 |
| 369 | 4863 | NPAT | RASSRST-pT-KKR--- | 1.0 |
| 370 | 4863 | NPAT | FRASSRS-pT-TKKR-- | 1.0 |
| 371 | 5128 | PCTK2 | FKRRLSL-pT-LRGSQT | 1.0 |
| 372 | 5339 | plectin | --KRERK-pT-SSKSSV | 1.0 |
| 373 | 5339 | plectin | KKRERKT-pS-SKSSVR | 1.0 |
| 374 | 5587 | PKD1 | KHTKRKS-pS-TVMK-- | 0.3 |
| 375 | 5587 | PKD1 | VHYTSKD-pT-LRKRHY | 3.0 |
| 376 | 5590 | pkc-zeta | KSIYRRG-pS-RRWR-- | 0.0 |
| 377 | 6840 | supervillin | NVMKRKF-pS-LRAAEF | 0.5 |
| 378 | 7074 | TIAM1 | RSASKRR-pS-RFSS-- | 2.0 |
| 379 | 8436 | serum | -EKIKRS-pS-LKKVDS | 3.0 |
| | | deprivation | | |
| | | response; | | |
| 380 | 8915 | BCL10 | EISCRTS-pS-RKRAGK | 4.0 |
| 381 | 9020 | NIK | -WKGKRR-pS-KARKKR | 0.8 |
| 382 | 9101 | ubiquitin | --RARRD-pS-LKKIEI | 1.0 |
| | | specific | | |
| | | protease 8 | | |
| 383 | 9148 | neurlized- | --ALRRP-pS-LRREAD | 0.5 |
| | | like | | |
| 384 | 9162 | dag kinase | -NRKKKR-pT-SFKRKA | 0.6 |
| | | iota | | |
| 385 | 9595 | PSCDBP | DDFLRRS-pS-SRRNRS | 1.0 |
| 386 | 9828 | p164-RhoGEF | PRLIRRG-pS-KKRPAR | 0.0 |
| 387 | 10123 | ARL7 | MILKRRK-pS-LKQK-- | 0.0 |
| 388 | 10969 | EBNA1BP2 | KRPGKKG-pS-NKRPGK | 1.0 |
| 389 | 23227 | MAST4 | MVRRSKK-pS-KKKESL | 0.5 |
| 390 | 23227 | MAST4 | --RMVRR-pS-KKSKKK | 0.2 |
| 391 | 25836 | IDN3 | EVSRPRK-pS-RKRVDS | 0.4 |
| 392 | 25865 | PKD2 | ARIIGEK-pS-FRRSVV | 0.2 |
| 393 | 26191 | Lyp | -SVILRP-pS-KSVKLR | 0.9 |
| 394 | 55357 | PARIS1 | EYLERRA-pS-RRRAV- | 0.2 |
| 395 | 55672 | FLJ20719 | KKRRGRR-pS-TKKRRR | 0.0 |
| 396 | 55672 | FLJ20719 | KRRGRRS-pT-KKRRRR | 0.0 |
| 397 | 57082 | AF15q14; | SKSQRRK-pS-LKLK-- | 0.0 |
| 398 | 57731 | spectrin, | EGGDRRA-pS-GRRK-- | 0.9 |
| | | beta, 4 | | |
| 399 | 65125 | WNK1 | EYRRRRH-pT-MDKDSR | 0.4 |
| 400 | 672 | BRCA1 | RLRRKSS-pT-RHIHAL | 1.0 |
| 401 | 1128 | M1 | KIPKRPG-pS-VHRTPS | 0.5 |
| | | muscarinic | | |
| | | receptor | | |
| 402 | 1196 | CLK2 | -RRRRRR-pS-RTFSRS | 0.0 |
| 403 | 1196 | CLK2 | RSRTFSR-pS-SSMK-- | 2.0 |
| 404 | 1196 | CLK2 | RTFSRSS-pS-MK---- | 2.0 |
| 405 | 1198 | CLK3 | --------pS-YRWKRR | 2.0 |
| 406 | 1198 | CLK3 | WKRRRSY-pS-REHEGR | 2.0 |
| 407 | 1612 | DAP-kinase | -FIKKRR-pT-KSSRRG | 1.0 |
| | | 1 | | |
| 408 | 1612 | DAP-kinase | KSSRRGV-pS-RE---- | 1.0 |
| | | 1 | | |
| 409 | 1794 | DOCK2 | SKKRTKR-pS-S----- | 2.0 |
| 410 | 2081 | ERN1 | RHSFSRR-pS-GV---- | 4.0 |
| 411 | 2596 | gap-43 | ASFRGHI-pT-RKKLKG | 0.2 |
| 412 | 2837 | Urotensin-2 | YRRSQRA-pS-FKRA-- | 0.0 |
| | | receptor | | |
| 413 | 2837 | Urotensin-2 | LARAYRR-pS-QRASFK | 0.1 |
| | | receptor | | |
| 414 | 3985 | LIMK-2 | -----KA-pT-TKKRTL | 2.0 |
| 415 | 3985 | LIMK-2 | ----KAT-pT-KKRTLR | 4.0 |
| 416 | 4171 | MCM2; | RHRSMRK-pT-FARYLS | 2.0 |
| 417 | 4171 | MCM2; | KTFARYL-pS-FRRD-- | 2.0 |
| 418 | 4763 | NF1 | QKQRSAG-pS-FKRNSI | 1.0 |
| 419 | 4820 | NKTR | RSYSRSR-pS-RG---- | 5.0 |
| 420 | 4863 | NPAT | NTQQFRA-pS-SRSTTK | 2.0 |
| 421 | 4863 | NPAT | TQQFRAS-pS-RSTTKK | 3.0 |
| 422 | 5587 | PKD1 | VKHTKRK-pS-STVMK- | 5.0 |
| 423 | 5587 | PKD1 | HTKRKSS-pT-VMK--- | 4.0 |
| 424 | 5587 | PKD1 | ---RVVQ-pS-VKHTKR | 1.0 |
| 425 | 6429 | SFRS4 | KSKDKRK-pS-RKRS-- | 0.2 |
| 426 | 6429 | SFRS4 | KRKSRKR-pS------- | 0.6 |
| 427 | 6429 | SFRS4 | RSRSRSK-pS-KDKRKS | 0.4 |
| 428 | 6429 | SFRS4 | RSRSRSR-pS-KSKDKR | 0.3 |
| 429 | 6429 | SFRS4 | ----RSR-pS-RSRSKS | 0.6 |
| 430 | 6429 | SFRS4 | --RSRSR-pS-RSKSKD | 0.6 |
| 431 | 6594 | SMARCA5 | ARIQRRI-pS-IKKA-- | 0.1 |
| 432 | 6650 | SOLH | APLRRRE-pS-MHVEQR | 0.0 |
| 433 | 7273 | titin | DKKQIRS-pS-KKYR-- | 2.0 |
| 434 | 7273 | titin | KDKRQLR-pS-SKKYK- | 0.7 |
| 435 | 8436 | serum | SSLKKVD-pS-LKK--- | 5.0 |
| | | deprivation | | |
| | | response; | | |
| 436 | 8567 | MADD | SVRQRRM-pS-LRDD-- | 1.0 |
| 437 | 8621 | CDC2L5 | SRSRHRL-pS-RSR--- | 0.1 |
| 438 | 8621 | CDC2L5 | -SSRHSR-pS-RSRHRL | 0.9 |
| 439 | 8621 | CDC2L5 | YSRRRSP-pS-YSRHSS | 0.3 |
| 440 | 8621 | CDC2L5 | SRHSRSR-pS-RHRLSR | 0.4 |
| 441 | 8899 | PRP4 | -RDRGRR-pS-RSRLRR | 0.1 |
| 442 | 8899 | PRP4 | RSRLRRR-pS-RS---- | 0.1 |
| 443 | 8899 | PRP4 | RGGRRRR-pS-RSKVKE | 0.0 |
| 444 | 8899 | PRP4 | TTKKRSK-pS-RSKERT | 0.4 |
| 445 | 8899 | PRP4 | DRGRRSR-pS-RLRRRS | 0.1 |
| 446 | 8899 | PRP4 | RLRRRSR-pS------- | 0.6 |
| 447 | 8899 | PRP4 | GRRRRSR-pS-KVKEDK | 0.0 |
| 448 | 9020 | NIK | -KKRKKK-pS-SKSLAH | 2.0 |
| 449 | 9020 | NIK | KKRKKKS-pS-KSLAHA | 1.0 |
| 450 | 9088 | Myt1 kinase | QLQPRRV-pS-FRGE-- | 1.0 |
| 451 | 9221 | nucleolar | -----EK-pT-KKKRGS | 1.0 |
| | | phospho- | | |
| | | protein p130 | | |
| 452 | 9221 | nucleolar | RGSYRGG-pS-ISV--- | 0.6 |
| | | phospho- | | |
| | | protein p130 | | |
| 453 | 9360 | cyclophilin | ---TEEK-pS-KKRKKK | 1.0 |
| | | G | | |
| 454 | 9590 | gravin | AGWRKKT-pS-FRKP-- | 0.4 |
| 455 | 9590 | gravin | -AGWRKK-pT-SFRKPK | 0.3 |
| 456 | 9595 | PSCDBP | -DDFLRR-pS-SSRRNR | 3.0 |
| 457 | 9934 | GPR105 | STSVKKK-pS-SRN--- | 2.0 |
| 458 | 9934 | GPR105 | TSVKKKS-pS-RN---- | 2.0 |
| 459 | 9934 | GPR105 | KSSRNST-pS-VKKKSS | 0.3 |
| 460 | 9934 | GPR105 | LKSSRNS-pT-SVKKKS | 2.0 |
| 461 | 9934 | GPR105 | -LKSSRN-pS-TSVKKK | 1.0 |
| 462 | 23031 | MAST3 | KRSRRRE-pT-QDR--- | 0.1 |
| 463 | 26191 | Lyp | VKLRSPK-pS------- | 4.0 |
| 464 | 55357 | PARIS1 | EYLERRA-pS-RRRAV- | 0.2 |
| 465 | 55762 | FLJ10891; | ARPKTRI-pS-NKYR-- | 0.8 |
| 466 | 56000 | nuclear | SPYNRKG-pS-FRKQ-- | 0.1 |
| | | RNA export | | |
| | | factor 3 | | |
| 467 | 57468 | solute | ITDESRG-pS-IRRK-- | 2.0 |
| | | carrier | | |
| | | family 12 | | |
| | | member 5 | | |
| 468 | 79142 | MGC2941; | PGDGEKR-pS-RIKKSK | 2.0 |
| 469 | 79142 | MGC2941; | KRSRIKK-pS-KKRK-- | 0.0 |
| 470 | 79877 | FLJ22955; | ARLMRRN-pS-LNRK-- | 0.0 |
| 471 | 94121 | slp4 | -RQGKRK-pT-SIKRDT | 1.0 |
| 472 | 94121 | slp4 | RQGKRKT-pS-IKRDTV | 0.4 |
| 473 | 9162 | dag kinase | NRKKKRT-pS-FKRKA- | 0.0 |
| | | iota | | |

### EXAMPLE 5

### Analysis of Different Kinases Using the Same Superset

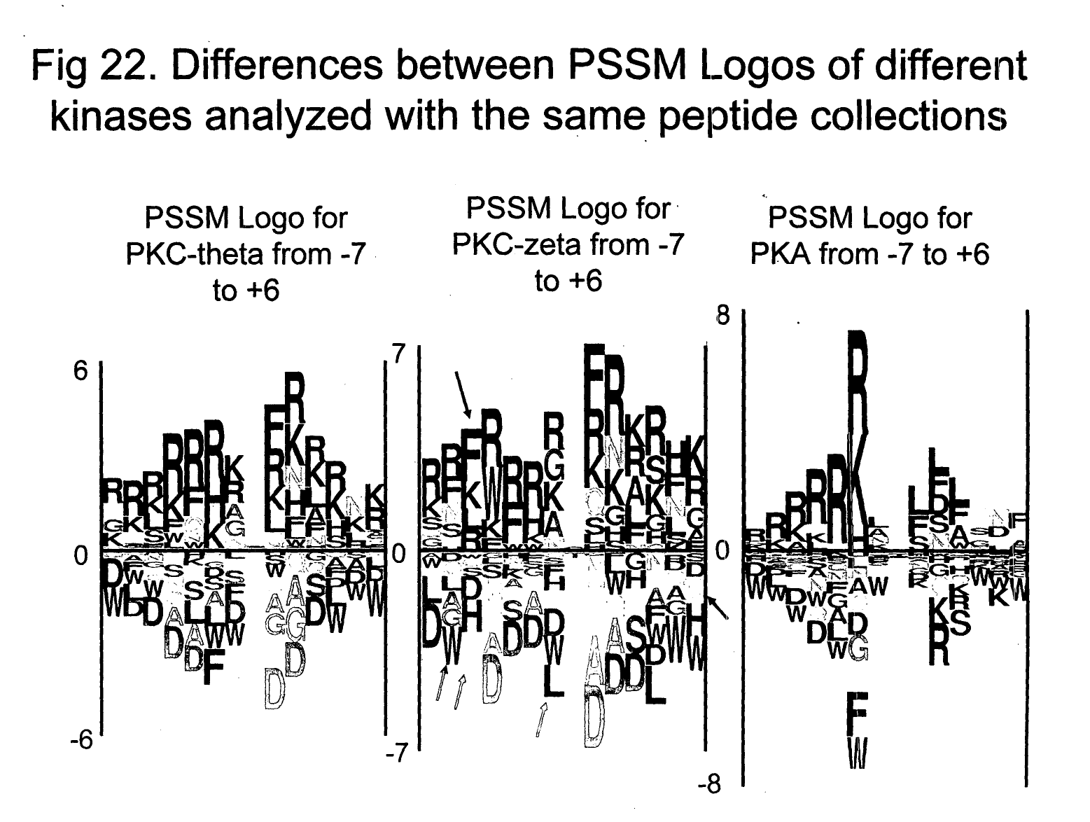

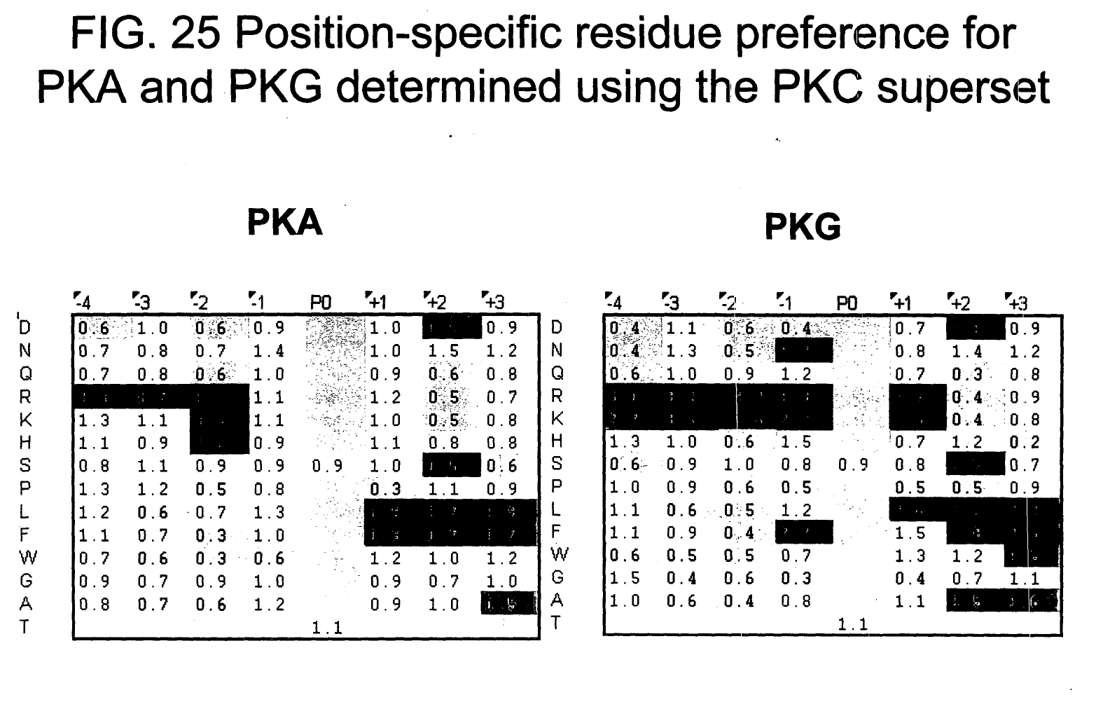

In many embodiments of the invention, the same superset of test peptides can be used to study the substrate specificity of a variety of different kinase enzymes. The anchor residue(s) and phosphorylatable residue in a test set (or superset, or collection) of peptides must be appropriate to the particular kinase whose specificity is being analyzed. However, a wide diversity of peptide sequences is available in the test sets, supersets, or collections of peptides provided by the invention. It is also fortunate that the results obtained to date indicate that there is sufficient similarity between the substrate specificities of different kinases that a single set (or superset, or collection) of peptide pools can be used to study the specificity of different kinases. Hence, for example, kinases of the protein kinase C family are sufficiently closely related that successful studies with other members of this family can be performed on the same or similar test sets of peptides. This was shown by studies that where one or both of the supersets of peptides designed for PKC were successfully used to analyze related kinases such as PKC-zeta, Protein Kinase A (PKA) and Protein Kinase G (PKG). See FIG. 22 and FIG. 25.

FIG. 22 shows PSSM Logos for PKC-zeta and PKA derived by analyzing those kinases with the same peptide supersets used for analysis of PKC-theta. Because the sequence of PKC-zeta is similar to the PKC-theta sequence, PKC-zeta was expected to have fundamental similarities in substrate specificity. Those expectations were confirmed by the PSSM Logo representation of the data. One of the most prominent differences between PKC-theta and PKC-zeta was the preference for a hydrophobic amino acid (e.g., phenylalanine, F) at P−5. This characteristic preference of PKC-zeta was confirmed using the methods of the invention and was further validated by previous tests (Nishikawa K, Toker A, Johannes F J, Songyang Z, Cantley L C. 1997. J Biol Chem 272:952-960). Similarly, PKA has a strong preference for positively charged residues in positions P−2 and P−3 (FIG. 22), as previously shown by Kreegipuu A, Blom N, Brunak S, Jarv J. 1998. Statistical analysis of protein kinase specificity determinants. FEBS Lett 430:45-50.)

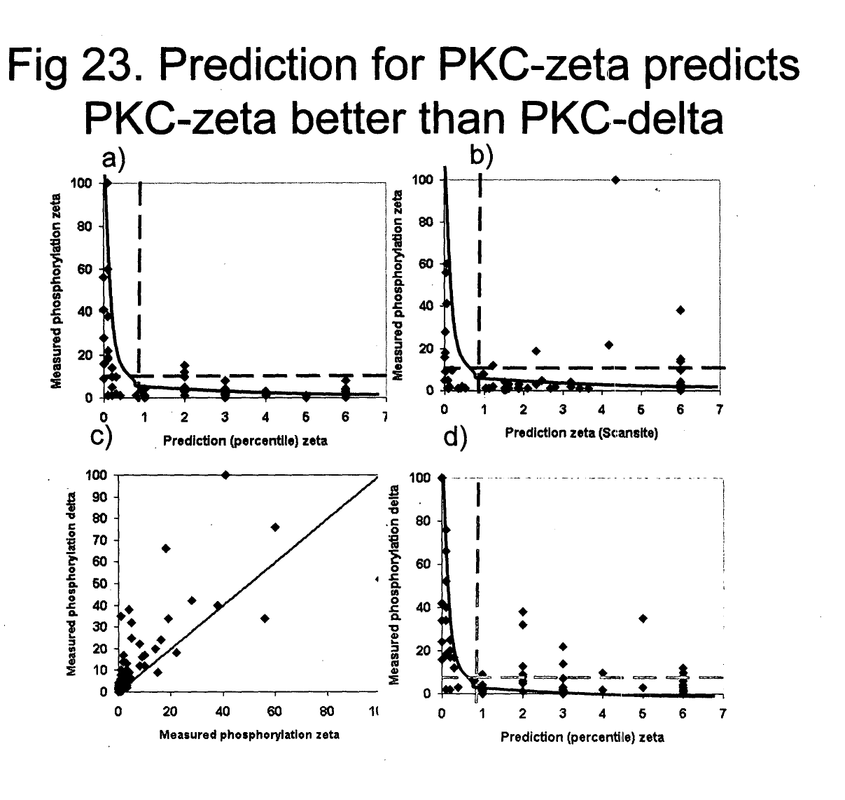

Predictions were made as to which amino acids would occupy what positions in the phosphorylation substrate recognized by PKC-zeta. These predictions were then tested by measuring PKC-zeta mediated phosphorylation of the same set of proteomic peptides that were tested for PKC-theta. The results for this testing are shown in FIG. 23 (panel a) and demonstrate that the PKC-zeta prediction was excellent. The quality of the prediction was affirmed by the comparison with the results of predictions by the Scansite for PKC-zeta (FIG. 23, panel b). Problems with the Scansite prediction were evident from the finding that the best peptide has a score of >4th percentile and several other of the better substrates also have scores >4^{th} percentile.

Given the similarity between the PSSM Logo for PKC-zeta and PKC-theta, it was possible that the good results for PKC-zeta and PKC-theta are redundant, and that nothing new has been learned from PKC-zeta. That possibility was addressed in two ways. First, the data were checked to ascertain whether PKC-delta/theta and PKC-zeta were equivalent in their phosphorylation of the set of proteomic peptides. Results in FIG. 23 (panel c) show that although there was a general correlation between the phosphorylation patterns of those different kinases, there were also substantial differences. Therefore, an analysis was performed on whether the PKC-zeta prediction would satisfactorily predict phosphorylation by PKC-delta. The results in FIG. 23 (panel d) demonstrate that PKC-zeta predictions would not. Thus predictions from the PKC-zeta PSSM predict well phosphorylation by PKC-zeta but not PKC-theta while predictions from the PKC-theta PSSM predict well phosphorylation by PKC-theta (and PKC-delta). These findings strongly validate the high degree of specificity provided by the methods of the invention.

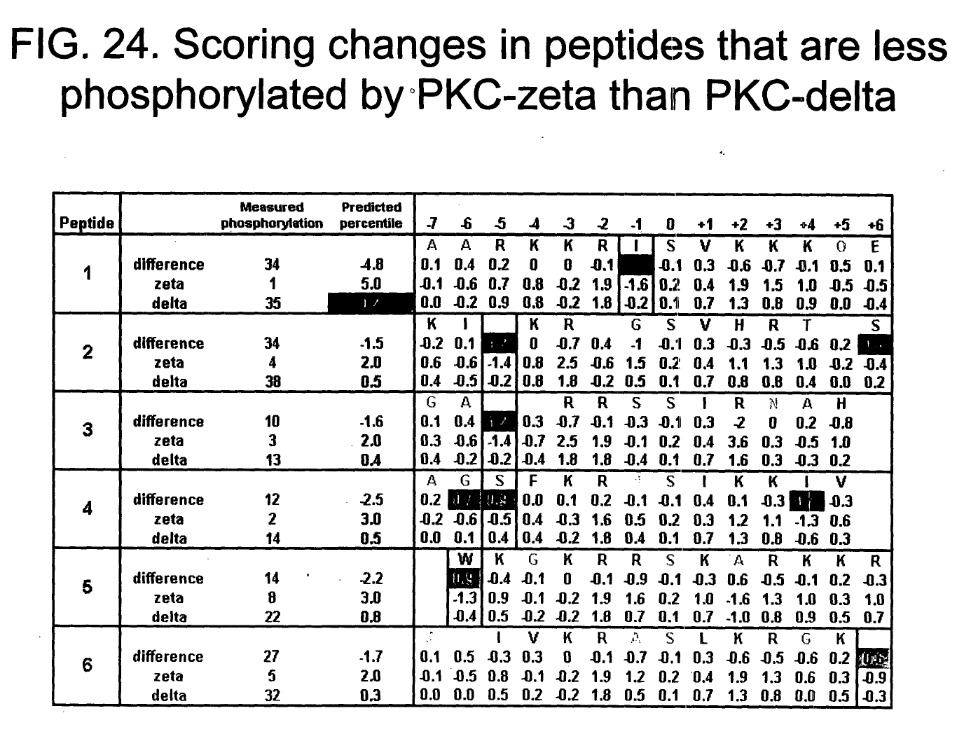

Further investigations were performed to ascertain what residues may account for differences between substrates in the predicted phosphorylation by PKC-theta and PKC-zeta. FIG. 24 provides a detailed analysis of the scoring for the six substrates whose behavior contributed most to the mismatch in FIG. 23, panel d (and corresponding match in FIG. 23, panel a). Scoring for those peptides with the PKC-theta and PKC-zeta predictions were tabulated. Residues that showed the biggest improvement in score with PKC-zeta relative to PKC-theta were identified (difference >0.5) and are highlighted in red. Better recognition by PKC-delta could be due to a favorable residue for PKC-delta recognition that is less favorable for PKC-zeta recognition (referred to herein as “control by favorable residue”), or to neutral residue for PKC-delta recognition being unfavorable for PKC-zeta recognition (“control by unfavorable residue”). The results indicate that much of the poorer recognition by PKC-zeta was due to at least one unfavorable residue. For example, the six biggest changes in score for each peptide have been boxed in black in FIG. 24. Five of those six changes are from a residue slightly unfavorable for PKC-theta to a residue very unfavorable for PKC-zeta. This is best illustrated by peptides 2 and 3, which have a proline at −5 that was slightly unfavorable for PKC-theta and very unfavorable for PKC-zeta. The strongly disfavored proline at −5 for PKC-zeta (but not for PKC-theta) can be seen in FIG. 22. This principle is similarly illustrated by the peptide 1, which has an isoleucine at P+1 (predicted as being disfavored based on the results for leucine with PKC-zeta, FIG. 22) and peptide 5, which has an W at P−5 (strongly disfavored by PKC-zeta, FIG. 22).

Control of kinase specificity by unfavorable residue(s) was also strongly suggested by the findings that PKA, PKC-theta and PKC-zeta all strongly disfavor proline at P+1 (FIG. 22). This contrasts sharply with the preferences of another major class of kinase, the proline-directed kinase, for which a Proline at P+1 is a critical residue. Thus, an important part of the reciprocal specificity between the basophilic kinases and the proline-directed kinases (such as CDK1) is that proline at P+1 was disfavored by the former and favored by the latter. Thus, “control by unfavorable residue” appears to be a major element in kinase specificity. This is important, because the methods of the invention can be very accurate at quantifying unfavorable recognition. Many of the prior art techniques may not be ideal for determining strength of unfavorable recognition; for example, the methods disclosed in U.S. Pat. No. 6,004,757 may be limited in doing so by reason of limitations in amino-acid sequencing.

### EXAMPLE 6

### Analysis of Mutant Kinases

In another embodiment, the methods of the invention can be used to analyze the substrate specificity of mutant kinases. A major strategy for analyzing protein structure and function involves deriving mutant constructs, expressing them, and determining how the mutation influences the function and/or specificity of the resulting mutant protein. Given the previous difficulty in assessing kinase specificity, there have been no prior studies that systematically analyze the specificities of mutant kinases. However, the methods of the invention can be used for this purpose.

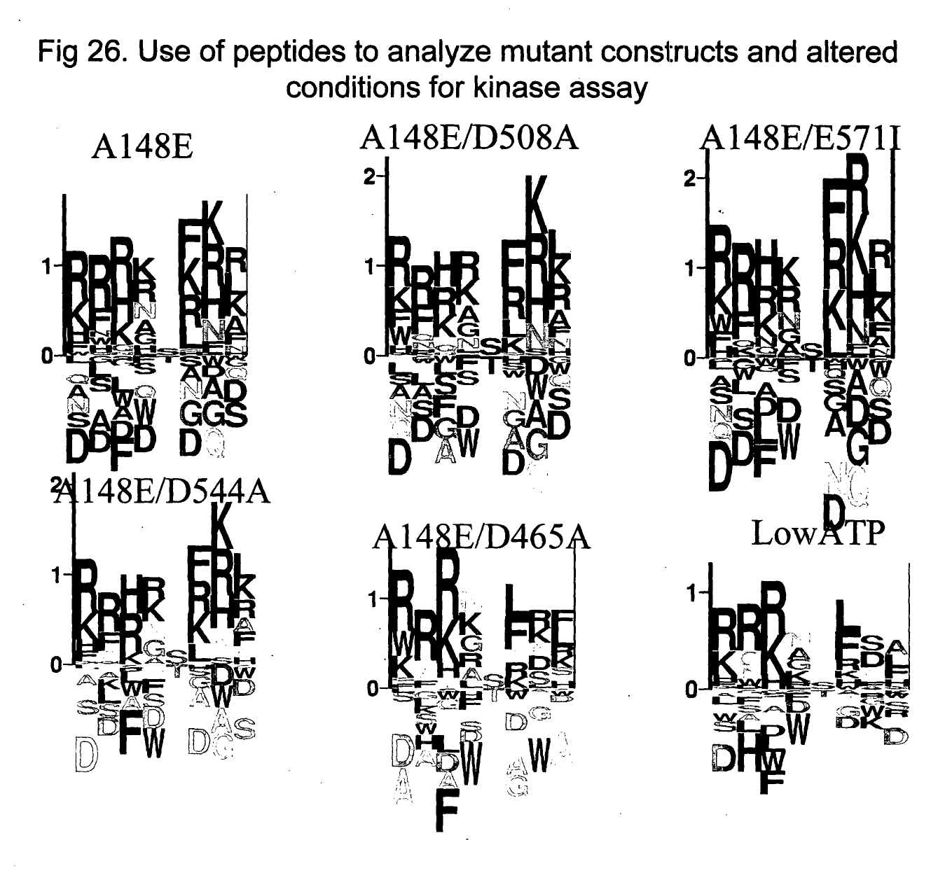

For example, more than ten mutant constructs of PKC-theta have been made and analyzed by the inventor using the present methods to ascertain what types of specificity changes occur. Results of some of the more informative constructs are shown as PSSM logos in FIG. 26. Because only changes in substrate specificity were assessed and not changes in auto-inhibition resulting from altered binding of pseudo-substrate, the parental construct PKC-theta was used that had been previously mutated to a constitutively active form by mutating the pseudo-substrate (A148E), shown in FIG. 26. Results are shown for four constructs in which acidic residue in the catalytic cleft has been mutated (FIG. 26).

The most striking finding amongst the constructs studied was deviation of construct D465A from the overall pattern of substrate specificities shared by wild type PKC-theta (FIG. A), constitutive active A148E (FIG. 26) and the three other mutant constructs derived from constitutive active A148E (D544A, D508A, E571I, FIG. 26). The differences observed in D465A specificity compared to other PKC-theta enzymes are: 1) the shapes of the PSSM Logo (i.e. relative height of individual columns) and 2) the general position of individual residues in particular columns.

Regarding the shape of the PSSM Logo, a feature absolutely conserved amongst constructs other than D465A was that the P+2 position was always the tallest. Usually the P+1 position was the second tallest and there was wobble as to which of the other positions was third tallest. However, mutant D465A was strikingly different. Position P+2 of the preferred substrate for the D465A mutant has dropped from the most prominent to one of the three least prominent and the P+1 position has likewise dropped in prominence. Taken together these data indicate that the D465A mutant has a marked reduction in reliance on the usual C-terminal residues that typically guide substrate specificity in all other kinase constructs.

A detailed understanding of kinase specificity requires understanding of the residues favored at each position. PSSM Logos (FIG. 26) also reveal that the strong preferences and lack of preferences of the wild type construct for residues at particular positions was typically conserved amongst most mutant kinase constructs. These generally include: 1) a preference for basic residues at each position; 2) an absolute preference for a hydrophobic residue that exceeds the preference for basic residues at the P+1 position (and occasionally P+3); 3) a strong disfavor for aspartic acid (‘D’) at most positions; 4) a strong dislike for hydrophobic residues at P−2; and 4) a strong disfavor for proline (‘P’) in a C-terminal position. As with the overall shape, D465A was also an outlier with regard to these preferences and disfavors. Note particularly the moderation, or reversal in preference for the typically disfavored ‘P’ and ‘D’ residues in the C-terminal positions of the substrate.

The marked changes in preference of the D465A mutant toward the C-terminal residues were not anticipated. However, it is known that the side chain of D465 coordinates with ATP. Consequently truncating the side chain of D465 would be expected to perturb some aspect of ATP binding or function. No major change in the Km for ATP, however, was revealed by analysis of the kinetic parameters for D465A. Therefore, ATP contact with the remainder of the ATP pocket within the enzyme may be sufficient for good binding in D465A. However, the conformation of the enzyme's N-lobe may be abnormal due to a lack of favorable interaction between the D465 side chain and other elements in the N-lobe. This incomplete closure would be expected to alter the “closed conformation” that the enzyme usually adopts during catalysis, and alter movement of alphaC towards the activation loop.

### EXAMPLE 7

### Analysis of Different Assay Conditions with Methods of the Invention

Tests were performed on a wild type kinase to examine whether low ATP concentrations would favor an ordered reaction in which a peptide binds first in the absence of ATP, and subsequent loading of ATP rapidly proceeds to catalysis. The PSSMLogo for such as assay is shown in FIG. 26. This PSSMLogo for low ATP reveals a distortion of shape that bears substantial resemblance to the D465A PSSMLogo. Specifically, there were decreases in height of the P+2 and P+3 columns that are even more marked that those observed with D465A. Moreover, like D465A, the low ATP profile has lost many of the characteristic preferences of the other constructs at these positions (see below).

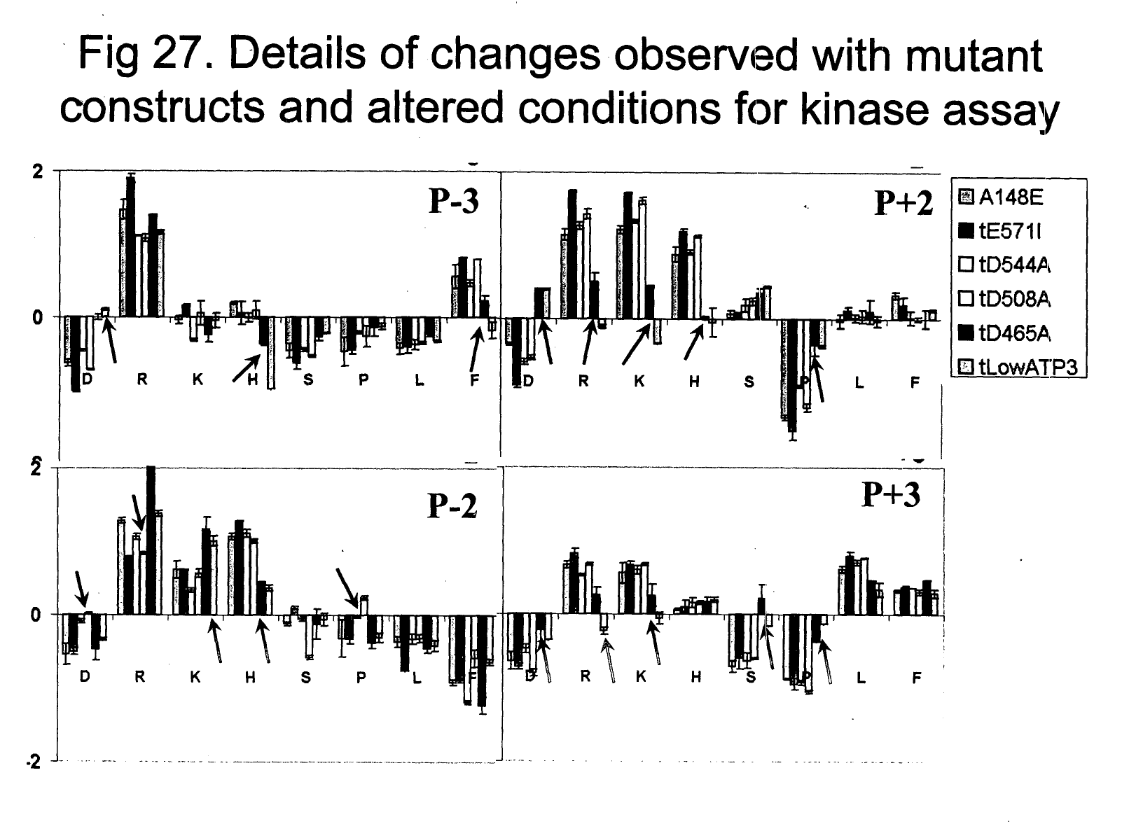

Visualization of D465A preferences at individual positions was facilitated by the graphical analysis shown in FIG. 27, which shows data for the eight most informative residues at four particularly informative positions. Positions P−2 and P−3 are shown in part because those are the peptide positions at which the greatest changes resulting from point mutations of acidic residues were anticipated. Positions P+2 and P+3 are shown because they are the location of many of the biggest changes in D465A and low ATP conditions. The most striking finding was the similarity in residue preference that occurs with D465A and low ATP, but not for other mutants. There were fifteen such changes, denoted with red arrows below the line in FIG. 27. Amongst these changes, five occur in the N-terminal P−2 and P−3 positions. Two of these N-terminal changes were ones that had been predicted, namely decreased preference for H at P−3 and decreased disfavor for D at P−3. The failure to see decreased preference for R or K at P−3 suggests that conformational flexibility allows binding of the P−3 substrate residue to residues other than D465 in the cleft (most likely D544 or D508).

The correlation between the D465A and low ATP changes in the C-terminal region of the substrate was striking. In almost all cases the changes in substrate preference observed for D465A involve neutralization of the strong preferences (either negative or positive) observed for related kinases. In contrast to D465A, changes in substrate preference for the other three point mutants are quite modest both in number and magnitude of change. However, some changes in substrate preference for the D508A mutant bear similarity to those found in D544A (denoted with blue arrows above the line in FIG. 27). Both have lost their disfavor for D at the P−2 position (consistent with repulsion by nearby residues). Both also show a modest decrease in preference for R, not only at P−2 but also at P−3.

The methods of the invention are therefore informative not only for studying the specificities of mutant kinase constructs, but also for analyzing changes in kinase specificity resulting from different assay conditions. It can be easily appreciated by one of skill in the art that the present methods would be useful in analyzing importance of other assay conditions, such as ion concentration (Ca++, Mg++, H+), and temperature. The present methods would also be useful in determining whether addition of other molecules to the assay influenced peptide specificity, for example by allosteric effects.

### EXAMPLE 8

### Further Understanding of Anchor Residues and their Variations in Test Sets

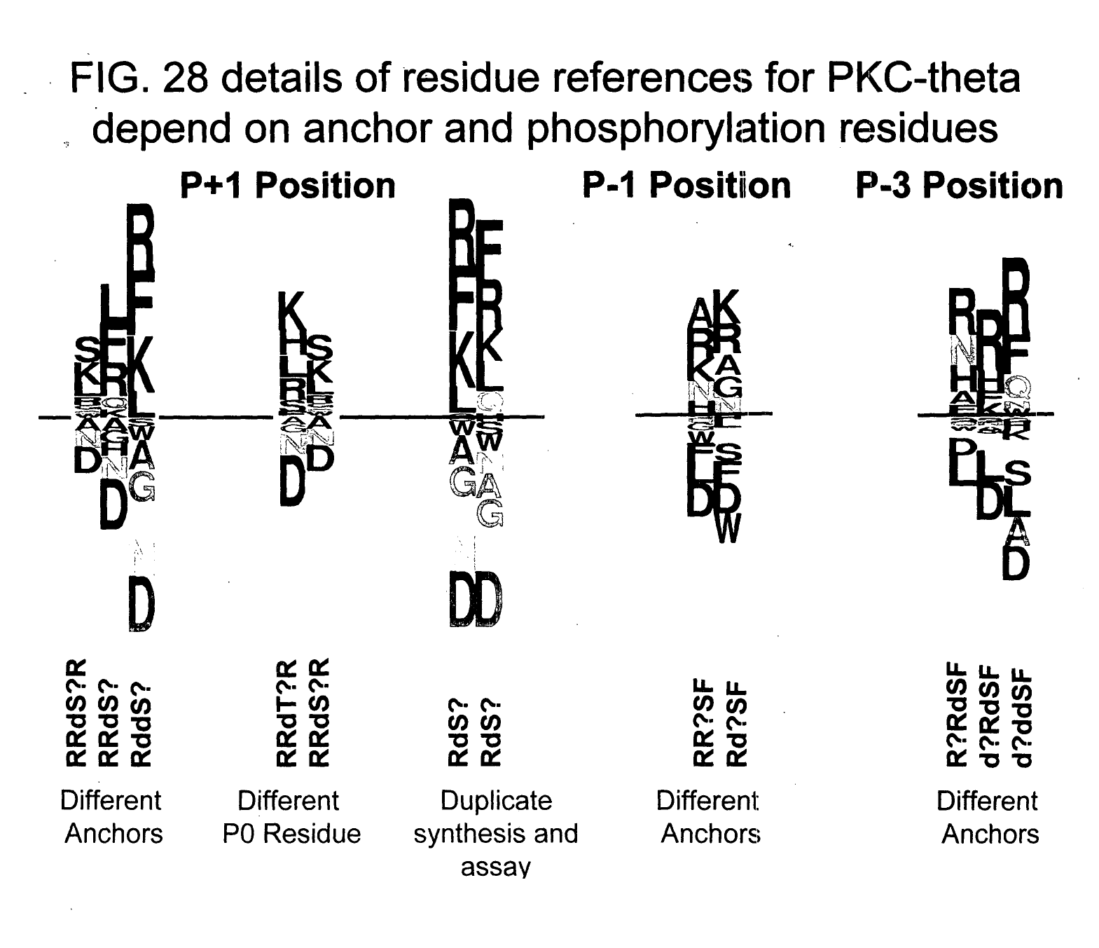

Understanding of substrate specificity usually requires understanding the residue preferences at every position close to the phosphorylation position. The problem related to establishing anchor positions is that positions that are chosen as anchor residues in a set cannot, by definition, also be query or variable positions in that set. For example, the peptide test set Rxx-S-F uses anchor residues at positions P−2 and P+1. Therefore, information on the P−2, P0 and P+1 positions cannot be obtained from the Rxx-S-F test set. In the embodiment shown in FIG. 2, the P−3, P0, and P+1 positions were analyzed by using diminished numbers of anchor residues. For example, for the P+1 test set, the anchor at P−3 was retained, but the P+1 position was used as the query position (variable residue). Note that the methods of the invention provide strategies for designing and using a variety of test sets that could determine information about the residue preference for PKC-theta at the P+1 position. FIG. 28 illustrates results with such varied test sets used for analysis of specificity of PKC-theta; each column of the PSSM logo represents results with a single test set and the symbolic representation of that set is shown below the column. Consider for example residue preference at the P+1 position, which our experience with the methods of the invention indicates is particularly important. Residue scores determined for that position vary depending on the number (and position) of the anchor residues used in the test set. Also note that the results differ significantly for test sets in which the phosphorylatable residue is T rather than S. For one skilled in the art, the methods of the invention provide many strategies to refine the definition of specificity for a kinase. For example, because the P+1 preferences for threonine phosphorylation differ from those for serine phosphorylation, one can create test sets analogous to those shown in FIG. 2, but using T as the phosphorylatable residue. Results with those peptides would allow more precise predictions, because they would be tailored specifically to relevant subsets of peptide substrates.

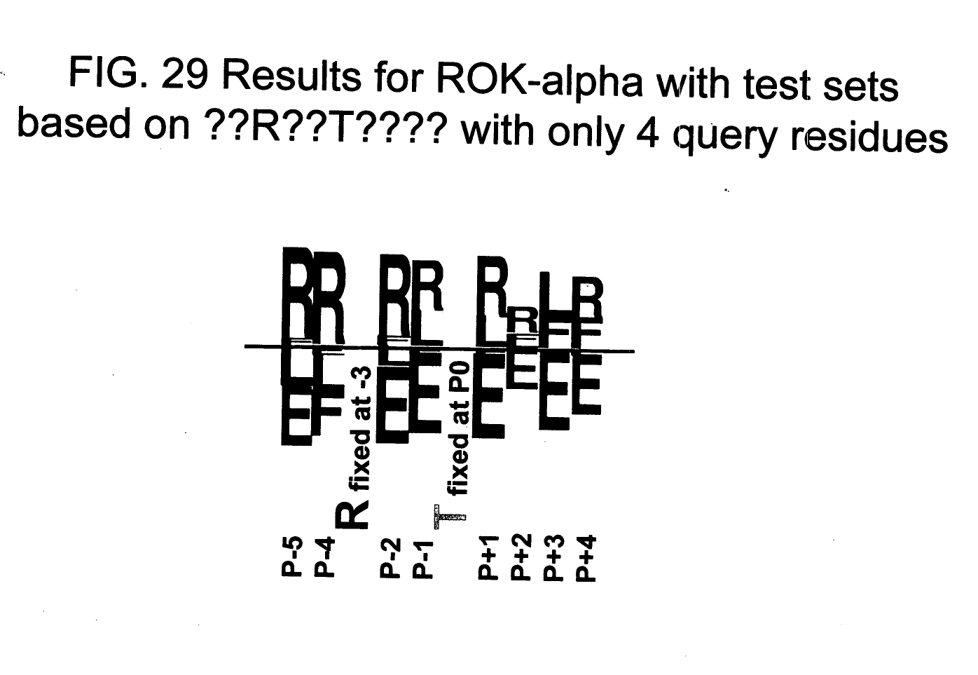

FIG. 29 illustrates results with another superset of test sets of peptide pools based on a single anchor residue of R at P−3 and threonine as the phosphorylatable residue. Results shown are for the kinase ROK-alpha, about which there is little general understanding of specificity in the literature. This superset is designed as a screening set to ascertain gross preferences from which to choose an additional anchor position. For that reason, it was most economical to only include 4 query residues: R, E, L and F, which our experience indicates are particularly important anchor residues. Even this limit analysis shows a strong overall preference for R, indicating ROK is clearly a “basophilic kinase”. The only position tested which has a dominant hydrophobic preference is P+3. One practiced in the art of this invention can appreciate that the third anchor position for a full test set of peptides should most likely be an ‘R’ at the P−4 or P−5 positions, where it has the strongest preference and where there are no other favorable residues.

### EXAMPLE 9

### Querying by Fixed Residue at Varied Positions Rather than by Varied Residue at Fixed Position

The large family of basophilic kinases has a preference for arginine (R) at many positions in the substrate (see for example, FIG. 8, FIG. 13, FIG. 22, and FIG. 29). Accordingly, arginine is a good candidate for an anchor residue at the high-scoring position(s). With this in mind, over-representation of arginine in anchor optimization sets used to assign anchor positions is a good first approach for an assay designed to assign anchor positions because the data indicate that arginine can markedly enhance the efficiency of phosphorylation when it is present in a peptide substrate for such kinases.

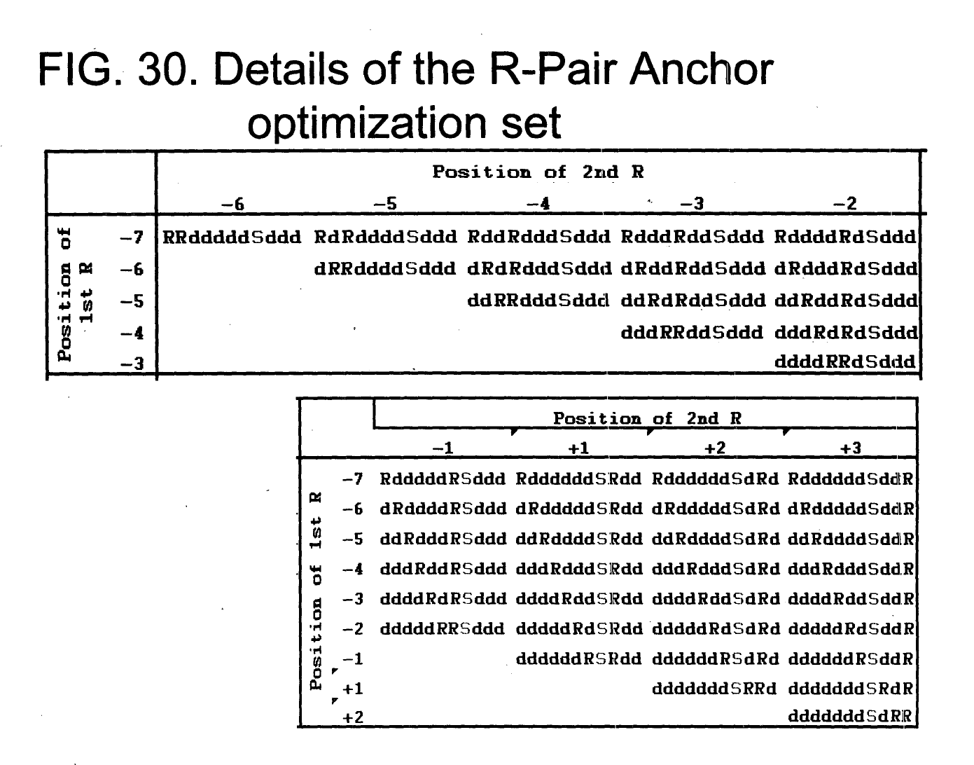

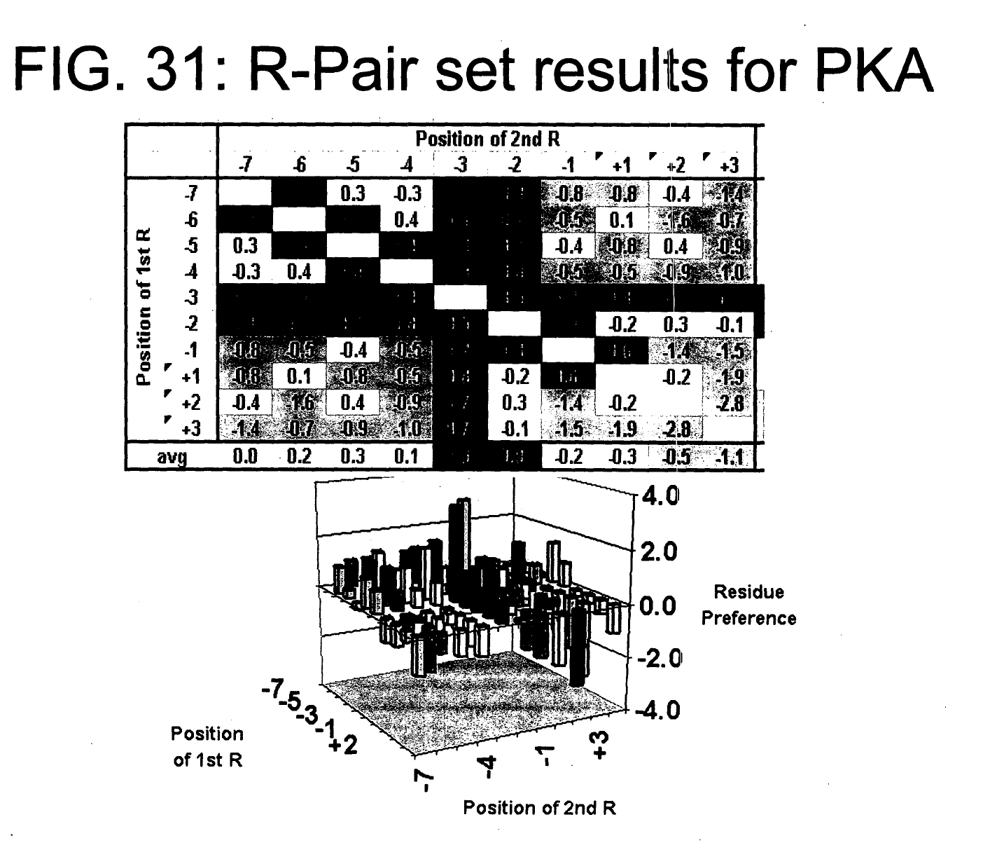

In this Example, an anchor optimization set referred to as an “R-pair set” was created to systematically evaluate the use of arginine in each position around P0 (in this set occupied by serine) from position P−7 to P+3. FIG. 30 shows the forty-five peptide sequences of this R-pair set. Results for the R-pair set using protein kinase A (PKA) are shown in FIG. 31. The results were calculated in a fashion similar to the sets described previously. Residue preference was calculated as follows:
[cpm for a peptide calculated as the geometric mean for replicate values]/[geometric mean cpm for all peptides in the set].
The position specific residue score was determined by calculating log₂ of the residue preference. An average score for arginine at each position was also calculated as the arithmetic average of the scores for all nine peptides that have a fixed arginine at the position. Inspection of the average score reveals that there PKA shows a strong overall preference for arginine at positions P−3 and P−2. Inspection of the results for individual peptides confirms that PKA most efficiently phosphorylates the individual degenerate peptide that has arginine fixed at both P−3 and P−2. These results for PKA are in agreement with a summary of the literature, for example with results obtained by the Tegge approach to determining optimal kinase substrates (Tegge W et al. 1995. Biochemistry 34:10569-10577).

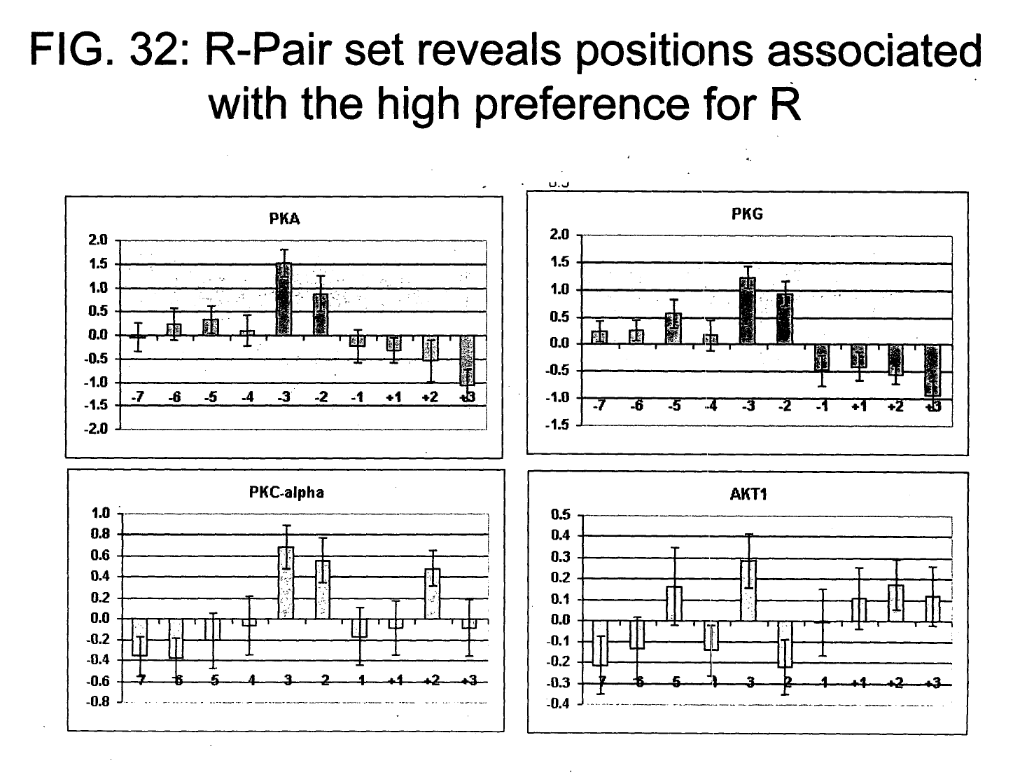

One simple way to summarize the results of studies with the R-pair set is to determine the geometric average preference for all peptide pools that have R at a given position. For example, in this embodiment, there are 9 peptide pools that have R at P−3 (see FIG. 30 and FIG. 31). The geometric average preference for R in those 9 pools is 1.5 (FIG. 32). Similar calculations for the other positions, results in the graph shown for PKA in FIG. 32 which likewise illustrates that PKA prefers R at P−2 and P−3.

Use of the R-pair set for anchor optimization with other kinases is likewise highly informative. For example, a comparison of the average position-specific scores for PKC-alpha and AKT1 with those described above for PKA is shown in FIG. 32. As shown in FIG. 32, PKC-alpha prefers arginine at P−3, P−2 and P+2. This is precisely the dominant positions at which the strongest preference for basic residues have been found in a summary of literature results for PKC (Kreegipuu A et al. 1998. FEBS Lett 430:45-50). Results from an R-pair analysis with AKT1 show that arginine is preferably placed at positions P−3 and P−5 (FIG. 32); these results are in agreement with findings from the literature (Obata T et al. 2000. J Biol Chem 275:36108-36115). Thus, the strategy provided herein for efficiently scanning for critical residues provides highly informative results. These residues are candidates for anchor residues for more complete degenerate residue sets. One key advantage of this particular set (and the approach of position scanning) is that it provides an impartial way to assess the most important position for R without introducing biases from other anchor residues.

### EXAMPLE 10

### Detection of Phosphorylation of SHP-1 in Whole Cells

Prediction of phosphorylation sites is ultimately most useful to understanding cellular physiology when it can be applied to facilitate identification of sites that are relevant in intact cells. Therefore, the invention provides strategies to extend the information provided from the previously illustrated in vitro studies. For example, strategies employed for analyzing phosphorylation of the SHP-1 protein are described herein. SHP-1 (also referred to as PTP1c, PTP-N6 and SHPTP-1) is a tyrosine phosphatase that is critical to regulation of many signaling responses, including the process of activation of T-lymphocytes by the T-cell receptor (Okumura M et al. 1995. Curr Opin Immunol 7:312-319; Kosugi A et al. 2001. Immunity 14:669-680). The functioning of SHP-1, in particular its phosphatase activity, is modified by phosphorylation. Important sites known to be phosphorylated include Y536 and Y564, both of which are close to the C-terminus of the molecule (Zhang Z et al. 2003. J Biol Chem 278:4668-4674).

SHP-1 has been shown to be a substrate for serine phosphorylation by PKC (Zhao Z et al. 1994. Proc Natl Acad Sci USA 91:5007-5011). Moreover, phosphorylation of SHP-1 by PKC results in decreased catalytic activity of SHP-1 (Brumell J H et al. 1997. J Biol Chem 272:875-882). Other investigators have shown that a closely related phosphatase, SHP-2, is phosphorylated on serine residues close to its C-terminus (Strack V et al. 2002. Biochemistry 41:603-608). These investigators (Strack V et al. 2002. Biochemistry 41:603-608) may have incorrectly inferred that SHP-1 was not phosphorylated by PKC because they looked only at mobility shifts of the SHP-1 that do not reliably detect many phosphorylation events. Of particular note, the previous studies have not identified the critical site of phosphorylation by PKC.

The phosphorylation of SHP-1 was analyzed using the methods provided herein, including the predictive algorithm for PKC-theta. Because phosphorylation by PKC-theta correlates highly with that for PKC-alpha and PKC-delta, these predictions have relevance at least for PKC-alpha and PKC-delta, and likely provide a generalized prediction for novel and classical PKCs.

Table 7 provides the predictions made by the methods of the invention for SHP-1 phosphorylation. For PKC phosphorylation using the fifth percentile as a conservative cutoff that will include all plausible candidate sites for PKC (See FIG. 9 and FIG. 11), only three sites in SHP-1 are predicted to be phosphorylated (sites Ser-591 SEQ ID NO 298, Ser-26 SEQ ID NO 299 and Ser-32, SEQ ID NO 300).

TABLE 7

Three Predicted PKC Phosphorylation sites in SHP-1
whose corresponding phosphopeptides bind best to pPKC antibody

Site

| | |
|---|---|
| Gene | Site Properties |

| | | | | | | | | |
|---|---|---|---|---|---|---|---|---|
| and | | | | | | | pPKC | Binding of |
| Protein | SEQ | Phospho peptide | | PKC- | PKC- | | antibody | pPKC |
| Name | ID NO | Sequence | P0 | Theta | Zeta | PKA | Score | antibody |

| | | | | | | | | |
|---|---|---|---|---|---|---|---|---|
| SHP-1 | 298 | ADKEKSKG-pS-LKRK---- | 591 | 2 | 8 | 10 | 4 | 100 |
| | 299 | LKGRGVHG-pS-FLARPSRK | 26 | 0.3 | 0.8 | 10 | 2 | 54 |
| | 300 | HGSFLARP-pS-RKNQGDFS | 32 | 2 | 2 | 20 | 3 | 39 |
| | 289 | MKNAHAKA-pS-RTSSKHKE | 553 | 8 | 8 | 10 | 2 | 18 |
| | 290 | RVILQGRD-pS-NIPGSDYI | 294 | 60 | 60 | 10 | 2 | 13 |
| | 291 | AHAKASRT-pS-SKHKEDVY | 556 | 10 | 20 | 30 | 3 | 12 |
| | 292 | KKKLEVLQ-pS-QKGQESEY | 528 | 30 | 30 | 90 | 2 | 11 |
| | 293 | PSEPGGVL-pS-FLDQINQR | 431 | 50 | 50 | 30 | 2 | 9 |
| | 294 | HAKASRTS-pS-KHKEDVYE | 557 | 8 | 7 | 2 | 1 | 7 |
| | 295 | PWTFLVRE-pS-LSQPGDFV | 138 | 40 | 20 | 7 | 3 | 5 |
| | 296 | KNQGDFSL-pS-VRVGDQVT | 42 | 10 | 20 | 50 | 3 | 3 |
| | 297 | PLNCSDPT-pS-ERWYHGHM | 107 | 60 | 60 | 90 | 2 | 0 |

The inventor has validated in vitro that a peptide comprising Ser-591 is phosphorylated by PKC (see SEQ ID NO 209, in Table 3). Tests were conducted to test whether SHP-1 is phosphorylated in vivo at Ser-591, Ser-26 or Ser-300. To do so, a strategy was employed that used a commercially available antibody from Cell Signaling Technology that is referred to as a phospho-PKC motif antibody (designated herein as pPKC Ab). (See U.S. Pat. No. 6,441,140 and Cell Signaling Technology Datasheet for ‘Phospho-(Ser) PKC Substrate Antibody’). Information from Cell Signalling Technology indicates that this antibody preparation may recognize a motif consisting of positively charged residue at P−2, a serine at P0, a hydrophobic residue at P+1 and a positively charged residue at P+2. Such antibodies can be used for detection of unknown proteins that contain phosphorylation sites conforming to the motif to which they bind. For example, phosphorylated proteins can be detected on two-dimensional gels with the pPKC Ab and the identity of these phosphorylated proteins can be confirmed by the observed molecular weight, isoelectric point and other information such as the predictive algorithms provided herein. Similarly, such detected proteins can be enriched by classical biochemical separations, and when sufficiently enriched, can be identified by mass spectrometry (Astoul E et al. 2003. J Biol Chem 278:9267-9275).

One basis for predicting whether the pPKC antibody can bind to a particular phosphorylation site is the extent of its conformity with the motif described for the antibody: [RK]x-pS-[FYILMV][RK]. Therefore for each candidate site in SHP-1, a score from 0 to 4 was calculated based on the number of matches of the sequence to that pattern. That “pPKC antibody score” is tabulated for pertinent SHP-1 sites in Table 7. So, for example, Ser-591 is the only site in SHP-1 that has a perfect score of 4.

Because these studies of SHP-1 were an early precedent-setting study, a rigorous approach was adopted in determining whether pPKC antibody binding included one or more of the three predicted SHP-1 sites. Phosphorylated peptides corresponding to the three best predicted PKC sites were synthesized (Table 7) in microtiter wells using a method of prior art described in U.S. Pat. No. 6,031,074. In addition, phosphorylated peptides corresponding to others sites in SHP-1 that match part of the motif detected by the pPKC antibody were also synthesized. Those phosphorylated peptides were then analyzed for reactivity with the pPKC antibody in an ELISA assay.

Among the phosphorylated peptides corresponding to sites in SHP-1, the best binding of pPKC antibody was detected with the phosphorylated peptide corresponding to Ser-591 (Table 7; pPKC antibody binding to other sites was normalized to the value obtained for Ser-581). Phosphorylated peptides corresponding to the other two PKC sites, Ser-26 and Ser-32 had distinctly lower but readily detectable binding. Other phosphorylated peptides exhibited low levels of binding that were not much above background.

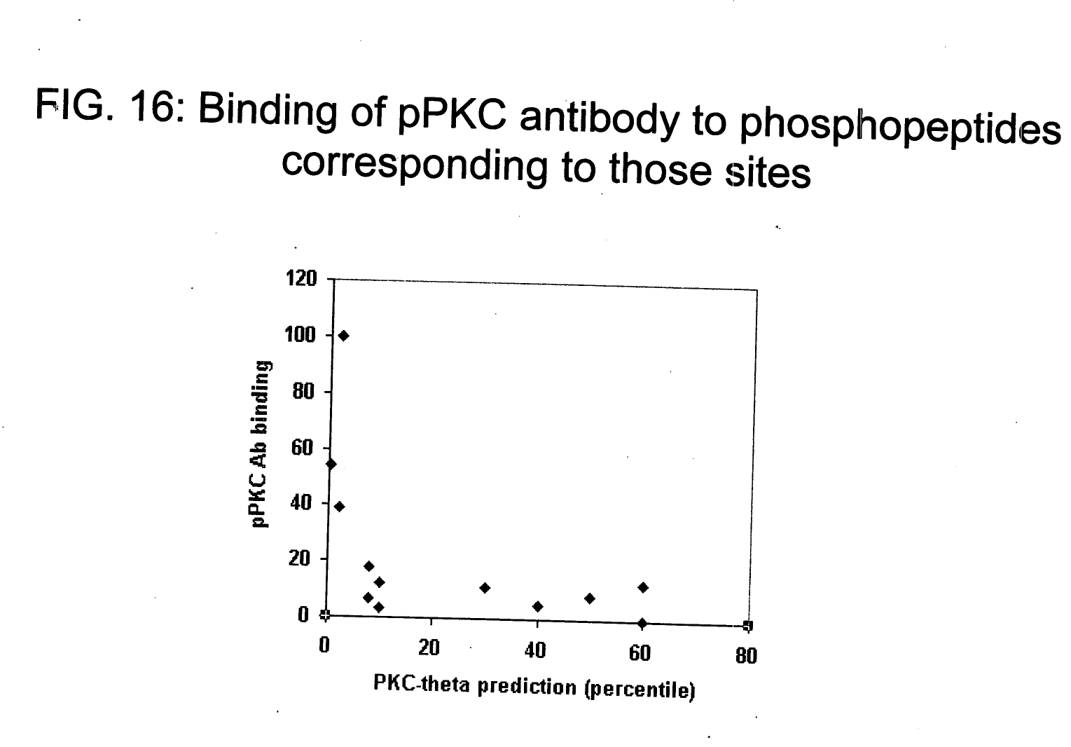

The correlation between the predictions of the invention for PKC and the pPKC antibody binding results to the corresponding phosphorylated peptides is shown in FIG. 16. As shown in FIG. 16, the sites predicted to be the best PKC sites are also the ones for which the corresponding phosphorylated peptide bind best to the antibody. Thus, a peptidyl sequence comprising Ser-591 of SHP-1 is a substrate for pPKC in vitro (Table 2, SEQ ID NO 209), is the only phosphorylated site in SHP-1 that perfectly matches the pPKC antibody motif, and is the phosphorylated site to which the pPKC antibody binds best. Thus, the site in SHP-1 has the properties that would be expected for the dominant site phosphorylated by PKC in vivo. The other two sites, Ser-26 and Ser-32 are likely secondary sites of PKC phosphorylation on SHP-1.

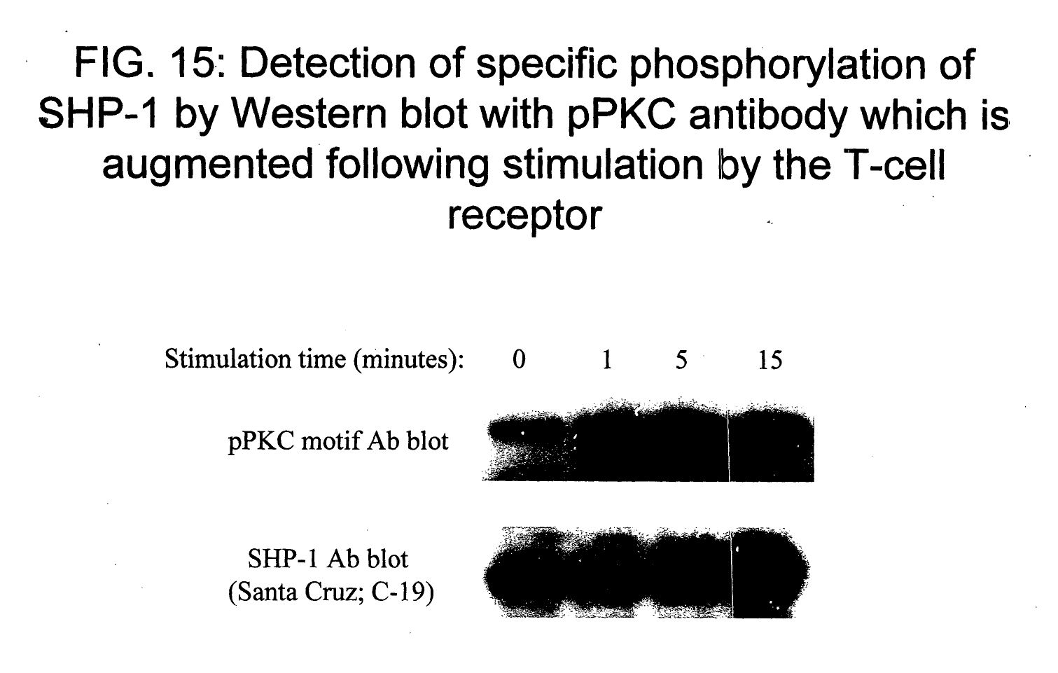

To test whether phosphorylation actually occurs at these sites in vivo, an antibody specific for the corresponding phosphorylated peptide can be used. However, because the identity of the relevant sites was previously unknown, no such specific antibodies were available in the prior art. The inventor therefore devised an alternative approach using the pPKC Ab. Although antibodies such as the pPKC Ab are poly-specific, they can be constrained to provide information on the phosphorylation state of a particular molecule such as SHP-1 by isolating the molecule of interest and then testing the antibody for reactivity with that isolated molecule. That strategy was implemented for SHP-1. In particular, SHP-1 was immunoprecipitated from the cell lysate of the cell line JURKAT with an anti-SHP-1 antibody (C-19; from Santa Cruz Biotechnologies) and protein G beads. The purified SHP-1 was separated by standard polyacrylamide gel electropheresis, transferred onto a membrane, and blotted with 2 different antibodies as shown in FIG. 15. Results from Western blotting with the anti-SHP-1 antibody (C-19 from Santa Cruz Biotechnologies) demonstrate that SHP-1 was successfully isolated and that it had a molecular weight of 64 kd, characteristic of SHP-1. That SHP-1 immunoprecipitate also reacted with the pPKC motif Ab, indicating the presence on SHP-1 of phosphorylated sites that conform to the motif recognized by the pPKC antibody.

FIG. 15 also includes information on JURKAT cells stimulated to activate SHP-1 via a T-cell receptor. Specifically, Jurkat T Ag cells were stimulated with CD3 antibody (clone 38.1, IgM ascites, 1:1000 Final) plus CD28 antibody (clone 9.3, sup, 1:1000 final) for different times, as indicated in FIG. 15. The amount of phosphorylated SHP-1, detected by intensity of the band on the pPKC antibody Western blot, increased markedly within the first minute following stimulation. These data demonstrate that the phosphorylation of SHP-1 at the sites recognized by the antibody is increased following T-cell receptor stimulation.

Thus, the sites on SHP-1 detected by the pPKC antibody (Table 7) are biologically relevant for immune cell responses (FIG. 15). Although the pPKC antibody is sufficient to identify these important sites, the pPKC antibody does not have desirable properties for easy detection of this biologically relevant change. Fortunately, now that these sites have been identified by the foregoing analysis, straightforward procedures can be used for making antibodies that are specific for those sites. For example, the inventors have raised such specific antibodies previously (Liu Y et al. 2002. Biochem J 361:255-65), hundreds of highly specific antibodies are available commercially, and many companies such as Anaspec offer packages of services to produce such antibodies. Such antibodies have much narrower specificity than the pPKC antibody and therefore would be useful for detection this phosphorylation without prior immunoprecipitation. Description of relevant strategies provided to a practioner of the art include those described in CURRENT PROTOCOLS IN CELL BIOLOGY, CHAPTER 16. ANTIBODIES AS CELL BIOLOGICAL TOOLS, UNIT 16.6 Production of Antibodies That Recognize Specific Tyrosine-Phosphorylated Peptides. In particular, methods available in the art include, purification of binding entities that bind specificity to the phosphorylated peptide; depletion of binding entities that cross-react on the non-phosphorylated peptide and depletion of binding entities that cross-react on the a distinct phosphopeptide. Therefore, for example, unlike the pPKC antibody which the manufacturer Cell Signaling Technology shows binds to the phosphorylated Ser-152 site in AFX (WKNpSIRH, SEQ ID NO: 229) and to the phosphorylated Ser-133 site in CREB (RRPpSYRK, SEQ ID NO 230), these antibodies provided by the invention would have substantially no binding to the phosphorylated Ser-152 site in AFX (WKNpSIRH, SEQ ID NO:229) or to the phosphorylated Ser-133 site in CREB (RRPpSYRK, SEQ ID NO:230).

### EXAMPLE 11

### Additional Examples of Proteins Predicted to Have Good PKC Phosphorylation Sites and Found to Bind pPKC Antibody by Western Blot

The usefulness of antibodies in implementing methods of the invention is further illustrated by studies of two additional proteins: LIMK-2 and MLK3. LIMK-2 and MLK3 ware identified as promising candidates for phosphorylation by PKC based on predictions for PKC-theta described herein and confirmation of that prediction by in vitro peptide phosphorylation (SEQ ID NO: 76 in Table 4 and SEQ ID NO: 121 in Table 5). To determine whether the pPKC Ab bound to predicted phosphorylated sites in MLK3 and LIMK2, a strategy was used that is complementary to the one shown in FIG. 7.

This strategy involved analysis of binding of pPKC Ab to peptides phosphorylated by PKC in vitro. Synthetic peptides chosen from those shown in Table 4 were subjected to phosphorylation by PKC-theta, Assay conditions were similar to those described herein, except that the phosphorylation reaction was for 30 minutes at 30° C. and then overnight at 4° C. The reaction mixture was applied to HB avidin-coated plates, the plates washed, and then pPKC Ab binding assayed. The results of these assays are summarized in Table 8.

TABLE 8

pPKC Antibody binds to peptides after phosphorylation by PKC-theta

pPKC Ab Signal

| | | | on peptide | on peptide | | |
|---|---|---|---|---|---|---|
| | | | without | after exposure | amount | Peptide |
| Gene | SEQ | | exposure to | to PKC-theta | dependent on PKC | phosphorylation |
| name | ID NO | Sequence | PKC-theta | phosphorylation | phosphorylation | by PKC-theta |
| MLK 3 | 76 | HVRRRRGTFKRS | 0.07 | 1.02 | 0.95 | 99 |
| | | KLRARD | | | | |
| LIMK-2 | 121 | LRRRSLRRSNSI | 0.02 | 1.13 | 1.11 | 57 |
| | | SKSPGP | | | | |
| ROC K2 | 75 | EEAEHKATKARL | 0.02 | 0.02 | 0.00 | 0 |
| | | ADK | | | | |

As shown in FIG. 8, the pPKC Ab bound to the peptides from LIMK-2 and from MLK3 after phosphorylation but not before. A control peptide is also shown, which is not phosphorylated by PKC and shows no change in binding to pPKC Ab after the peptide was exposed to PKC-theta.

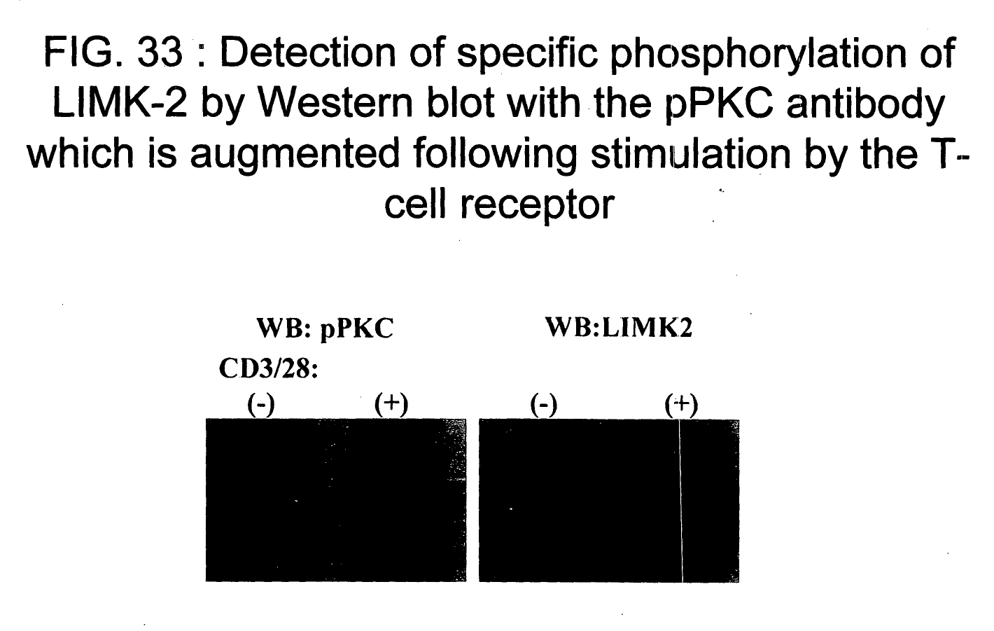

The question of in vivo relevance of LIMK-2 phosphorylation was addressed using the strategy used above for SHP-1. LIMK-2 was immunoprecipitated with anti-LIMK2 antibody H-78 purchased from Santa Cruz Biotechnologies, separated by one-dimensional PAGE and analyzed by Western blot. As shown in FIG. 33, the Western blot revealed immunoprecipitation of LIMK-2 from T-lymphocytes before and after T-cell receptor stimulation. Western blot reactivity with the pPKC antibody showed a signal indicating phosphorylation of LIMK-2; of note, the pPKC signal was observed only on the sample from T-cell receptor stimulation cells, indicating that pPKC-detected phosphorylation of LIMK-2 occurred during T-cell receptor stimulation.

Similar studies were performed with the protein MLK3. Jurkat T Ag cells (10 million) were stimulated with CD3 (clone 38.1, IgM ascites, 1:1000 Final) plus CD28 (clone 9.3, sup, 1:1000 final), or with PMA (200 ng/ml) for 5 minutes. MLK3 was immunoprecipitated from the cell lysate with anti-MLK3 Ab (H-300; from Santa Cruz) and protein G beads. Part of the immunoprecipitated MLK3 was blotted with pPKC Motif Ab, and part blotted with MLK3 Ab. As shown in FIG. 34 MLK3 has strong reactivity with the pPKC antibody both before and after stimulation of JURKAT cells. The prediction phosphorylation site at Ser-477 on MLK3 corresponds to one of the very best in the entire human proteome analyzed, and JURKAT cells is a partially activated transformed cell line. The binding of pPKC antibody therefore most likely reflects phosphorylation of MLK3 present even in unstimulated cells.

Note that the sequences shown in Tables 2-7 represent sequences of peptides. In general they match sequences of the corresponding human protein(s) except where the protein(s) sequence comprises a cysteine that cysteine has been replaced with an alanine in the peptide sequence.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A test set for characterizing substrate specificities of kinases comprising at least two peptide pools, wherein substantially every peptide in each of the peptide pools comprises one phosphorylatable amino acid position, one query amino acid position, at least one anchor amino acid position, and at least one degenerate amino acid position, and wherein:
each peptide of every peptide pool has an identical phosphorylatable amino acid that can be phosphorylated by a kinase at the phosphorylatable amino acid position;
the query amino acid position is at a defined position relative to the phosphorylatable amino acid position within every peptide of every peptide pool but a query amino acid's identity at the query amino acid position is systematically varied from one peptide pool to the next peptide pool within the test set of peptide pools;
each anchor amino acid position is at a defined position relative to the phosphorylatable amino acid position within every peptide of every peptide pool and each anchor amino acid position has an identical anchor amino acid at that anchor amino acid position within every peptide of every peptide pool;
each degenerate amino acid position within every peptide of every peptide pool is occupied by an amino acid from a defined mixture of amino acids; and
the query amino acid position is not adjacent to an anchor amino acid position or the query amino acid position is not adjacent to the phosphorylatable amino acid position in any peptide pool of the test set.

2. The test set of claim 1, wherein at least one anchor amino acid is arginine.

3. The test set of claim 1, wherein at least one anchor amino acid is proline.

4. The test set of claim 1, wherein at least one anchor amino acid is phenylalanine.

5. The test set of claim 1, wherein an anchor amino acid position is located one position C-terminal to the phosphorylatable amino acid position.

6. The test set of claim 5, wherein proline is the anchor amino acid at the anchor amino acid position located one position C-terminal to the phosphorylatable amino acid position.

7. The test set of claim 5, wherein glutamine is the anchor amino acid at the anchor amino acid position located one position C-terminal to the phosphorylatable amino acid position.

8. The test set of claim 5, wherein arginine is the anchor amino acid at the anchor amino acid position located one position C-terminal to the phosphorylatable amino acid position.

9. The test set of claim 5, wherein phenylalanine is the anchor amino acid at the anchor amino acid position located one position C-terminal to the phosphorylatable amino acid position.

10. The test set of claim 1, wherein an anchor amino acid position is located three positions N-terminal to the phosphorylatable amino acid position.

11. The test set of claim 10, wherein arginine is the anchor amino acid at the anchor amino acid position located three positions N-terminal to the phosphorylatable amino acid position.

12. The test set of claim 1, wherein every peptide in each of the peptide pools comprises less than four anchor amino acids.

13. The test set of claim 1, wherein at least one degenerate position in each peptide pool in the test set is occupied by a defined mixture of more than five amino acids.

14. The test set of claim 13, wherein the defined mixture comprises all natural amino acids.

15. The test set of claim 13, wherein the defined mixture comprises all natural amino acids except cysteine.

16. The test set of claim 13, wherein each amino acid's relative abundance in the defined mixture is approximately that amino acid's human proteome relative abundance.

17. The test set of claim 13, wherein the defined mixture of amino acids comprises proline.

18. The test set of claim 13, wherein the defined mixture of amino acids comprises arginine.

19. The test set of claim 1, wherein the test set has at least four peptide pools and each of the four peptide pools have a different query amino acid.

20. The test set of claim 1, wherein the query amino acid position is two positions N-terminal to the phosphorylatable amino acid position.

21. The test set of claim 1, wherein the query amino acid position is two positions C-terminal to the phosphorylatable amino acid position.

22. The test set of claim 1, wherein one query amino acid is proline.

23. The test set of claim 1, wherein one query amino acid is arginine.

24. The test set of claim 1, wherein each peptide pool is a soluble mixture of peptides.

25. The test set of claim 24, wherein substantially every peptide is linked to biotin.

26. The test set of claim 1, wherein substantially every peptide of every peptide pool is attached to a solid support.

27. A test set for characterizing substrate specificities of kinases comprising at least two peptide pools, wherein substantially every peptide in each of the peptide pools comprises one phosphorylatable amino acid position, one query amino acid position, and at least one degenerate amino acid position, and wherein:
each peptide of every peptide pool has an identical phosphorylatable amino acid that can be phosphorylated by a kinase at the phosphorylatable amino acid position;
the query amino acid position is at a defined position relative to the phosphorylatable amino acid position within every peptide of every peptide pool but a query amino acid's identity at the query amino acid position is systematically varied from one peptide pool to the next peptide pool within the test set of peptide pools;
each degenerate amino acid position within every peptide of every peptide pool is occupied by an amino acid from a defined mixture of amino acids;
the query amino acid position is not adjacent to the phosphorylatable amino acid position in any peptide pool of the test set.

28. A test set for characterizing substrate specificities of kinases comprising at least two peptide pools, wherein every peptide in each of the peptide pools comprises one phosphorylatable amino acid position, one query amino acid, at least one anchor amino acid position, and at least one degenerate amino acid position, and wherein:
each peptide of every peptide pool has an identical phosphorylatable amino acid that can be phosphorylated by a kinase at the phosphorylatable amino acid position;
every peptide of every peptide pool has an identical query amino acid but the position of the query amino acid relative to the phosphorylatable amino acid position is systematically varied from one peptide pool to the next peptide pool within the test set of peptide pools;
each anchor amino acid position is at a defined position relative to the phosphorylatable amino acid position within every peptide of every peptide pool and each anchor amino acid position has an identical anchor amino acid at that anchor amino acid position within every peptide of every peptide pool;
each degenerate amino acid position within every peptide of every peptide pool is occupied by an amino acid from a defined mixture of amino acids.

29. The test set of claim 28, wherein there are at least three peptide pools.

30. The test set of claim 28, wherein there the query amino acid is arginine.

31. A binding entity whose binding differentiates between a defined peptide having any one of SEQ ID NO: 76, 79, 81, 82, 87, 89-94, 97, 98, 100, 102, 104, 105, 108, 110, 112, 113, 115, 117, 121, 124, 125, 127-134, 136, 138, 139, 143-145, 148-153, 156, 160, 163-180, 182-194, 196-206, 208-211, 213-216 and the corresponding defined peptide after phosphorylation by PKC-theta, and wherein the binding entity has substantially no binding to a phosphorylated peptide having SEQ ID NO: 229 (WKN-pS-IRH).

32. The binding entity of claim 31, wherein the binding entity binds less efficiently to the defined peptide than to the defined peptide after phosphorylation by PKC-theta.

33. The binding entity of claim 32, wherein the binding entity also has substantially no binding to a phosphorylated peptide having SEQ ID NO 230 (RRP-pS-YRK).

34. The binding entity of claim 32, wherein the defined peptide comprises SEQ ID NO:76 (HVRRRRGTFKRSKLRARD).

35. The binding entity of claim 32, wherein the defined peptide comprises SEQ ID NO:121 (LRRRSLRRSNSISKSPGP).

36. The binding entity of claim 32, wherein the defined peptide comprises SEQ ID NO:209 (DKEKSKGSLKRK).

37. The binding entity of claim 31, wherein the binding entity is a polypeptide or a mixture of polypeptides sharing a similar binding specificity.

38. The binding entity of claim 31, wherein the binding entity is an antibody, an antibody fragment or a mixture thereof.

39. The binding entity of claim 31, wherein the binding entity binds more efficiently to the defined peptide than to the defined peptide after phosphorylation by PKC-theta.

40. The binding entity of claim 31, wherein the defined peptide is part of a protein.

41. A binding entity whose binding differentiates between a defined phosphorylated peptide having any one of SEQ ID NO:298-347, 349-473 and a non-phosphorylated peptide that differs from the defined peptide by substitution of Ser for the pSer or substitution of a Thr for the pThr, and wherein the binding entity has substantially no binding to a phosphorylated peptide having SEQ ID NO: 229 (WKN-pS-IRH).

42. The binding entity of claim 40, wherein the binding entity binds more efficiently to the defined phosphorylated peptide than to the defined non-phosphorylated peptide.

43. The binding entity of claim 41, wherein the defined phosphorylated peptide comprises SEQ ID NO:298.

44. The binding entity of claim 41, wherein the defined phosphorylated peptide comprises SEQ ID NO:299 or 300.

45. The binding entity of claim 41, wherein the defined phosphorylated peptide comprises SEQ ID NO:313 or 314.

46. The binding entity of claim 41, wherein the defined phosphorylated peptide comprises SEQ ID NO:361 or 362.

47. The binding entity of claim 41, wherein the defined phosphorylated peptide comprises any one of SEQ ID NO:301-310.

48. The binding entity of claim 41, wherein the defined phosphorylated peptide comprises any one of SEQ ID NO:311-320.

49. The binding entity of claim 41, wherein the defined phosphorylated peptide comprises any one of SEQ ID NO:321-330.

50. The binding entity of claim 41, wherein the defined phosphorylated peptide comprises any one of SEQ ID NO:331-342.

51. The binding entity of claim 41, wherein the defined phosphorylated peptide comprises any one of SEQ ID NO:343-347, 349-362.

52. The binding entity of claim 41, wherein the defined phosphorylated peptide comprises any one of SEQ ID NO:363-382.

53. The binding entity of claim 41, wherein the defined phosphorylated peptide comprises any one of SEQ ID NO:383-473.

54. The binding entity of claim 40, wherein the binding entity binds less efficiently to the defined phosphorylated peptide than to the defined non-phosphorylated peptide.

55. The binding entity of claim 40, wherein the defined phosphorylated peptide is part of a protein.

56. A method for characterizing substrate specificities of kinases comprising:
contacting each peptide pool in at least two test sets of peptide pools with ATP and a kinase;
quantifying the amount of phosphorylation in each peptide pool; and
comparing the amount of phosphorylation in each peptide pool with the amount of phosphorylation in at least one other peptide pool;
wherein substantially every peptide in each of the peptide pools comprises one phosphorylatable amino acid position, one query amino acid position, at least one anchor amino acid position, and at least one degenerate amino acid position, and wherein:
each peptide of every peptide pool has an identical phosphorylatable amino acid that can be phosphorylated by a kinase at the phosphorylatable amino acid position;
the query amino acid position is at a defined position relative to the phosphorylatable amino acid position within every peptide of every peptide pool but a query amino acid's identity at the query amino acid position is systematically varied from one peptide pool to the next peptide pool within the test set of peptide pools;
each anchor amino acid position is at a defined position relative to the phosphorylatable amino acid position within every peptide of every peptide pool and each anchor amino acid position has an identical anchor amino acid at that anchor amino acid position within every peptide of every peptide pool; and
each degenerate amino acid position within every peptide of every peptide pool is occupied by an amino acid from a defined mixture of amino acids

57. The method of claim 56, wherein quantifying the amount of phosphorylation comprises determining a total amount of labeled phosphate incorporated into each peptide pool.

58. The method of claim 56, wherein quantifying the amount of phosphorylation comprises determining a total amount of phosphorylated peptide in each peptide pool with an antibody specific for a phosphorylated peptide.

59. The method of claim 56, wherein the method further comprises placing a value for each amount of phosphorylation into a matrix relating amino acid position and amino acid identity with the amount of phosphorylation.

60. The method of claim 56, wherein the matrix is used to predict preferred substrate peptide sequences for the kinase.

61. A computer readable medium comprising computer-executable instructions, wherein the computer-executable instructions comprise conversion of input data into quantitative values specifying a preference value for each of a plurality of amino acids at each defined position in a substrate peptide for a kinase, wherein:
the input data comprises sequence and phosphorylation data for a test set of peptides comprising at least two peptide pools, wherein every peptide in each of the peptide pools comprises one phosphorylatable amino acid position, and one query amino acid position, wherein:
each peptide of every peptide pool has an identical phosphorylatable amino acid that can be phosphorylated by a kinase at the phosphorylatable amino acid position;
the query amino acid position is at the defined position relative to the phosphorylatable amino acid position within every peptide of every peptide pool but a query amino acid's identity at the query amino acid position is systematically varied from one peptide pool to the next peptide pool within the test set of peptide pools;
a preference value for a particular amino acid at the defined position is substantially determined from the amount of phosphorylation of the peptide pool wherein that particular amino acid is the query residue and the query position is located at the defined position.

62. The computer readable medium of claim 61, wherein a ratio between (the preference value for one amino acid) and (the preference value for a second amino acid) is generally proportional to a ratio between (the amount of phosphorylation of the peptide pool in which the first amino acid is the query amino acid) and (the amount of phosphorylation of the peptide pool in which the second amino acid is the query amino acid).

63. The computer readable medium of claim 61, wherein the difference between (the preference value for one amino acid) and (the preference value for a second amino acid) is generally proportional to a logarithmic transformation of the ratio between (the amount of phosphorylation of the peptide pool in which the first amino acid is the query amino acid) and (the amount of phosphorylation of the peptide pool in which the second amino acid is the query amino acid).

64. The computer readable medium of claim 61, wherein the instructions further comprise inputting one or more peptide sequences and predicting a likelihood of phosphorylation of the one or more peptide sequences of said kinase.

65. A method for visual display of amino acid or nucleotide sequence preferences comprising a series of stacks of single letter symbols for amino acids or nucleotides, wherein
each stack represents a position in a peptide or a nucleic acid sequence;
each symbol's height is proportional to the absolute value of a quantitative parameter that is positive for favored amino acids or nucleotides and negative for disfavored amino acids or nucleotides;
each symbol's position within the stack is sorted from bottom to top in ascending value by the quantitative parameter.

66. A computer readable medium having computer-executable instructions for performing a method of visually displaying amino acid or nucleotide sequence preferences, the method comprising:
representing a position in a peptide or a nucleic acid sequence with a stack of single letter symbols for amino acids or nucleotides; and
displaying a linear array of one or more stacks of letter symbols wherein each letter symbol's height is proportional to the absolute value of a quantitative parameter that is positive for favored amino acids or nucleotides and negative for disfavored amino acids or nucleotides and wherein each letter symbol's position within the stack is sorted from bottom to top in ascending order by the value of the quantitative parameter.

67. A computer readable medium having computer-executable instructions of claim 66, wherein the symbols are single letter codes for amino acids.

68. A computer readable medium having computer-executable instructions of claim 66, wherein the sequence preferences relate to kinase specificity.
